(19) United States
(12) Patent Application Publication
(10) Pub. No.: US 2012/0082666 A1
(43) Pub. Date: Apr. 5, 2012

(54) ANTIBODY COMPOSITIONS AND METHODS OF USE

(76) Inventors: Xiaocheng Chen, Foster City, CA (US); Mark Dennis, San Carlos, CA (US); Becket Feierbach, Belmont, CA (US); Ashley Fouts, Menlo Park, CA (US); Isidro Hotzel, Brisbane, CA (US); Bing Li, Foster City, CA (US)

(21) Appl. No.: 13/248,998

(22) Filed: Sep. 29, 2011

Related U.S. Application Data
(60) Provisional application No. 61/504,056, filed on Jul. 1, 2011.
(60) Provisional application No. 61/387,725, filed on Sep. 29, 2010.
(60) Provisional application No. 61/387,735, filed on Sep. 29, 2010.

(51) Int. Cl. A61K 39/42; C12N 15/13; C12P 21/02; C12N 5/10; C12N 1/15; C12N 1/19; C07K 16/08; A61P 31/22

(52) U.S. Cl. 424/133.1; 530/389.4; 530/388.3; 530/388.15; 530/387.3; 424/159.1; 424/147.1; 424/142.1; 536/23.53; 435/328; 435/339; 435/419; 435/254.11; 435/254.2; 435/69.6

(57) ABSTRACT

The invention provides compositions comprising anti-gH antibodies and anti-Complex I antibodies as well as methods of using the same.

### RELATED APPLICATIONS

This application claims the benefit of U.S. Provisional Application Nos. 61/387,735 and 61/387,725, both filed on Sep. 29, 2010, and U.S. Provisional Application No. 61/504,056, filed Jul. 1, 2011, all of which are incorporated by reference herein in their entireties.

### REFERENCE TO SEQUENCE LISTING SUBMITTED AS A TEXT FILE VIA EFS-WEB

A sequence listing is submitted concurrently with the specification as an ASCII formatted text file via EFS-Web, with a file name of “P4680R1US.txt”, a creation date of Sep. 15, 2011, and a size of 200,277 bytes. The sequence listing filed via EFS-Web is part of the specification and is hereby incorporated by reference in its entirety herein.

### FIELD OF THE INVENTION

The present invention relates to anti-Complex I and anti-gH antibodies and methods of using the same.

### BACKGROUND

Human cytomegalovirus (HCMV) is a β-herpesvirus and is also known as human herpesvirus-5 (HHV-5). Other species of cytomegalovirus (CMV) exist which infect additional mammals such as murine CMV (MCMV), guinea pig CMV (GPCMV), simian CMV (SCCMV), rhesus CMV (rhCMV) and chimpanzee CMV (CCMV). HCMV is a common herpesvirus that infects nearly 50% of the U.S. population. For the vast majority of human infected individuals, HCMV infection is asymptomatic. However, in conditions of illness, and immune suppression (e.g., HIV infection, drug-induced immune suppression in transplant patients) HCMV reactivation or primary infection causes a variety of clinical manifestations such as mononucleosis, hepatitis, retinitis, pneumonia, blindness and organ failure. In addition, in the setting of pregnancy, the acquisition of primary CMV infection, though of little consequence to the mother, can have severe clinical consequences in the developing fetus.

Congenital HCMV infection is of particular importance as many children born to mothers infected during pregnancy become infected in utero and suffer devastating clinical disease. In the United States and Europe, 126,000 women have primary HCMV infection during pregnancy and approximately 40,000 of the babies born to these mothers have congenital infection. In the U.S., 1 in 750 children are born with or develop disabilities due to HCMV infection, including: mental retardation, hearing loss, vision loss, organ defects, and growth defects. Congenital HCMV infection is the most common infectious cause of fetal abnormalities. After primary infection of a pregnant woman has occurred, there is currently no approved therapy for the prevention or treatment of fetal infection. Thus, there is a great need in the art to find compositions and methods to prevent congenital HCMV infection.

In 2005, Nigro and colleagues published a study in which human CMV hyperimmune globulin (HIG) was administered to expectant mothers with primary HCMV infection (Nigro et al. (2005) New Engl. J. Med. 353:1350-1362). In one arm of the study only 1 of the 31 infants born to HCMV-infected mothers were born with disease while 7/14 (50%) of children born to untreated women were born with HCMV disease. Id.

During pregnancy, HCMV can spread from the infected mother to the fetus via the placenta. The placenta, which anchors the fetus to the uterus, contains specialized epithelial cells, stromal fibroblast cells, endothelial cells, and specialized macrophages. The HCMV viral surface contains various viral glycoprotein complexes that have been shown to be required for infection of the specific cell types found in the placenta. A complex of CMV glycoproteins containing gH/gL and UL128, UL130 and UL131 (herein referred to as “Complex I”) is specifically required for infection of endothelial cells, epithelial cells and macrophages. A complex of CMV glycoproteins containing gH/gL and gO (herein referred to as “Complex II”) is specifically required for infection of fibroblasts. HIG has been shown to block viral entry into all four of the placental cells that are susceptible to HCMV infection.

Due to the difficulty of preparing and widely distributing HIG and the reluctance of physicians and the medical community to use human blood products, particularly in pregnant women, it would be most beneficial to create a composition comprising a monoclonal antibody or monoclonal antibodies that could protect fetuses from congenital HCMV infection. No monoclonal antibody composition to date has been developed for the prevention of maternal-fetal transmission of CMV. Lanzavecchia and Macagno have disclosed naturally-occurring antibodies that were isolated from the immortalized B cells of infected patients that bind to a conformational epitope resulting from the combination of UL130 and UL131 or a combination of UL128, UL130 and UL131 that neutralizes CMV transmission (U.S. Patent Publication Nos. 2008/0213265 and 2009/0081230). Shenk and Wang have disclosed antibodies that bind to proteins of Complex I (U.S. Pat. No. 7,704,510). Funaro et al. also disclose neutralizing antibodies to CMV in U.S. Patent Publication No. 2010-0040602. Additionally, an anti-gH monoclonal antibody, MSL-109 was tested in humans in two patient populations, allogenic bone marrow transplant recipients and patients with AIDS and CMV retinitis (Drobyski et al., Transplantation 51:1190-1196 (1991); Boeckh et al., Biol. Blood Marrow Transplant. 7:343-351 (2001); and Borucki et al., Antiviral Res. 64:103-111 (2004) without success.

There remains a need in the art to develop monoclonal antibodies for preventing HCMV infection, including congenital HCMV infection.

### SUMMARY

The invention provides isolated antibodies which specifically bind to HCMV Complex I. In certain embodiments, the anti-Complex I antibodies of the invention comprise six HVRs: (a) an HVR-H1 comprising the amino acid sequence of SEQ ID NO:6; (b) an HVR-H2 comprising the amino acid sequence of SEQ ID NO:7; (c) an HVR-H3 comprising the amino acid sequence of SEQ ID NO:8; (d) an HVR-L1 comprising the amino acid sequence of SEQ ID NO:9; (e) an HVR-L2 comprising an amino acid sequence selected from the group consisting of SEQ ID NOs:10-19; and (f) an HVR-L3 comprising the amino acid sequence of SEQ ID NO:20. The antibodies may further comprise a light chain variable domain framework FR1 comprising the amino acid sequence of SEQ ID NO:43 and an FR2 comprising the amino acid sequence of SEQ ID NO:44. In additional embodiments, the anti-Complex I antibodies of the invention comprise three heavy chain hypervariable regions (HVR-H1, HVR-H2 and HVR-H3) and three light chain hypervariable regions (HVR-L1, HVR-L2 and HVR-L3), wherein: (a) HVR-H1 comprises the amino acid sequence of SEQ ID NO:6; (b) HVR-H2 comprises the amino acid sequence of SEQ ID NO:7; (c) HVR-H3 comprises the amino acid sequence of SEQ ID NO:8; (d) HVR-L1 comprises the amino acid sequence of SEQ ID NO:9; (f) HVR-L3 comprises the amino acid sequence of SEQ ID NO:20; and (e) HVR-L2 and the first amino acid of the light chain variable domain framework FR3 comprises the amino acid sequence of SEQ ID NO:21.

In particular embodiments, the anti-Complex I antibody comprises (a) a V_{H} comprising the amino acid sequence of SEQ ID NO:45, or SEQ ID NO:46, or SEQ ID NO:47; and (b) a V_{L} comprising the amino acid sequence of SEQ ID NO:48 or SEQ ID NO:49. Such antibodies may further comprise a light chain variable domain framework FR3 comprising the amino acid sequence of SEQ ID NO:41 and an FR4 comprising the amino acid sequence of SEQ ID NO:42.

In some embodiments, the anti-Complex I antibody comprises a V_{H} sequence having at least 95% sequence identity to the amino acid sequence of SEQ ID NO:45, or SEQ ID NO:46, or SEQ ID NO:47 and a V_{L} sequence having at least 95% sequence identity to the amino acid sequence of SEQ ID NO:48 or SEQ ID NO:49. In some embodiments, the antibody comprises a V_{H} comprising the amino acid sequence of SEQ ID NO:45, or SEQ ID NO:46, or SEQ ID NO:47. In some embodiments, the anti-Complex I antibody comprises a V_{L} comprising the amino acid sequence of SEQ ID NO:48 or SEQ ID NO:49. In some embodiments, the anti-Complex I antibody comprises a V_{H} sequence of SEQ ID NO:45 or SEQ ID NO:46 and a V_{L} sequence of SEQ ID NO:49.

The invention also provides isolated antibodies which specifically bind to HCMV gH.

In some embodiments, the anti-gH antibody of the invention comprises three heavy chain hypervariable regions (HVR-H1, HVR-H2 and HVR-H3) and three light chain hypervariable regions (HVR-L1, HVR-L2 and HVR-L3), wherein:

(a) HVR-H1 comprises the amino acid sequence of SEQ ID NO:71;

(b) HVR-H2 comprises an amino acid sequence selected from SEQ ID NO:72, SEQ ID NO:73 SEQ ID NO:74 and SEQ ID NO:93;

(c) HVR-H3 comprises the amino acid sequence of SEQ ID NO:75;

(d) HVR-L1 comprises the amino acid sequence of SEQ ID NO:76;

(e) HVR-L2 comprises the amino acid sequence of SEQ ID NO:77; and

(f) HVR-L3 comprises the amino acid sequence of SEQ ID NO:78.

In some embodiments, the anti-gH antibody comprises an HVR-H2 comprising the amino acid sequence of SEQ ID NO:93, wherein the amino acid at position 6 of SEQ ID NO:93 is selected from the group consisting of Ser, Thr, Asn, Gln, Phe, Met, and Leu, and the amino acid at position 8 of SEQ ID NO:93 is selected from the group consisting of Thr and Arg.

In certain embodiments, the anti-gH antibody comprises an HVR-H2 comprising an amino acid sequence of SEQ ID NO:72, SEQ ID NO:73 or SEQ ID NO:74.

In other embodiments, the anti-gH antibody comprises an HVR-H2 comprising the amino acid sequence of SEQ ID NO:94 wherein the sequence comprises an amino acid at position 54 (of SEQ ID NO:94) selected from the group consisting of Ser, Thr, Asn, Gln, Phe, Met, and Leu. In some embodiments, the antibody further comprises an amino acid at position 56 selected from the group consisting of Thr and Arg.

The invention also provides anti-gH antibodies having a V_{H} sequence that is at least 95% identical in amino acid sequence to SEQ ID NO:94 wherein the sequence comprises amino acid Asn54, Ser54, Thr54, Gln54, Phe54, Met54, or Leu54 and/or Arg56. In certain embodiments, the antibody comprises a V_{H} comprising an amino acid sequence selected from SEQ ID NO:87, SEQ ID NO:88 and SEQ ID NO:89. In some embodiments, the VH comprises an amino acid sequence that is 95% identical to SEQ ID NO:94 wherein the sequence contains an amino acid at position 54 selected from Asn54, Ser54, Thr54, Gln54, Phe54, Met54, or Leu54 and/or an Arg at position 56 (Arg56); and (b) a VL sequence having at least 95% sequence identity to the amino acid sequence of SEQ ID NO:90. In certain embodiments, the VH comprises an amino acid sequence selected from SEQ ID NO:87, SEQ ID NO:88 and SEQ ID NO:89. In some embodiments, the VL comprises the amino acid sequence of SEQ ID NO:90. In certain embodiments, the antibody comprises a VH sequence of SEQ ID NO:89 and a VL sequence of SEQ ID NO:90.

In certain embodiments the antibodies of the invention specifically bind to HCMV Complex I on the surface of HCMV and neutralize HCMV at an EC90 of 0.1 μg/ml or less. In certain embodiments, the isolated anti-Complex I antibodies of the invention specifically bind to HCMV Complex I on the surface of HCMV and neutralize 50% of HCMV at an antibody concentration of 0.05 μg/ml, 0.02 μg/ml, 0.015 μg/ml, 0.014 μg/ml, 0.013 μg/ml, 0.012 μg/ml, 0.011 μg/ml, 0.010 μg/ml, 0.009 μg/ml, 0.008 μg/ml, 0.007 μg/ml, 0.006 μg/ml, 0.005 μg/ml, 0.004 μg/ml, 0.003 μg/ml, 0.002 μg/ml, 0.001 μg/ml, 0.0009 μg/ml, 0.0008 μg/ml, 0.0007 μg/ml or less (e.g., at an antibody concentration of 10⁻⁸M, 10⁻⁹M 10⁻¹⁰ M, 10⁻¹¹ M, 10⁻¹² M, 10⁻¹³M, or lower).

In certain embodiments, isolated anti-gH antibodies of the invention specifically bind to HCMV gH. The antibodies bind to gH on the surface of HCMV and neutralize HCMV at an EC90 of 1 μg/ml or less. Isolated anti-gH antibodies of the invention bind to gH on the surface of HCMV and neutralize 50% of HCMV at an antibody concentration of 0.1 μg/ml, 0.09 μg/ml, 0.08 μg/ml, 0.07 μg/ml, 0.06 μg/ml, 0.05 μg/ml, 0.04 μg/ml, 0.03 μg/ml, 0.02 μg/ml, 0.015 μg/ml, 0.014 μg/ml, 0.013 μg/ml, 0.012 μg/ml, 0.011 μg/ml, 0.010 μg/ml, 0.009 μg/ml, 0.008 μg/ml, 0.007 μg/ml, 0.006 μg/ml, 0.005 μg/ml, 0.004 μg/ml, 0.003 μg/ml, 0.002 μg/ml, 0.001 μg/ml or less (e.g., at an antibody concentration of 10⁻⁸M, 10⁻⁹M 10⁻¹⁰ M, 10⁻¹¹ M, 10⁻¹² M, 10⁻¹³M, or lower).

The antibodies of the invention may be monoclonal antibodies, including, for example, human, humanized or chimeric antibodies. The invention also provides for antibody fragments that specifically bind HCMV gH and/or Complex I.

In particular embodiments, the antibody that specifically binds HCMV Complex I and/or gH is a full length IgG1 antibody.

The invention also provides isolated nucleic acid encoding the antibodies that specifically bind HCMV Complex I and/or gH. The invention also provides host cells comprising the nucleic acid encoding such antibodies.

The invention further provides a method of producing an antibody comprising culturing the host cells containing the nucleic acid encoding the antibody that specifically binds Complex I and/or gH so that the antibody is produced. The method may further comprise recovering the antibody from the host cell.

The invention also provides a pharmaceutical formulation comprising an anti-Complex I antibody, or an anti-gH antibody, or a combination of an anti-Complex I antibody and an anti-gH antibody and a pharmaceutically acceptable carrier. The pharmaceutical formulation of each antibody may be separate or combined. The pharmaceutical formulation may further comprise an additional therapeutic agent (e.g., ganciclovir, foscarnet, valganciclovir and cidofovir).

The invention also provides compositions comprising an anti-Complex I antibody, or an anti-gH antibody, or a combination of an anti-Complex I antibody and an anti-gH antibody. The composition comprising each antibody may be separate or combined. The composition may further comprise an additional therapeutic agent (e.g., ganciclovir, foscarnet, valganciclovir and cidofovir).

The invention also provides compositions comprising an anti-Complex I and/or an anti-gH antibody for use in inhibiting, treating or preventing HCMV infection. In some embodiments, the use is for inhibiting, treating or preventing congenital HCMV infection or HCMV infection in a tissue or organ transplant recipient for which the transplanted tissue, organ or the donor is or has been infected with HCMV. Additional embodiments include uses in which the transplant recipient has previously been infected with HCMV and is at risk of reactivation. In certain embodiments the tissue or organ transplant recipient is seronegative for HCMV infection. In certain embodiments the composition comprising the antibody which binds HCMV gH is separate from the composition comprising the antibody which binds HCMV Complex I.

Compositions comprising the antibodies of the invention may also be used in the manufacture of a medicament. The medicament may be for use in the treatment, inhibition or prevention of HCMV infection, such as, for example, inhibiting, preventing or treating congenital HCMV infection or HCMV infection in an organ or tissue transplant recipient for which the transplanted organ, tissue or the donor is or has been infected with HCMV. In additional embodiments the transplant recipient has previously been infected with HCMV and is at risk of reactivation. In certain embodiments, the medicament may further comprise an additional therapeutic agent (e.g., ganciclovir, foscarnet, valganciclovir and cidofovir). In certain embodiments the organ or tissue transplant recipient is seronegative for HCMV infection. In certain embodiments the composition comprising the antibody which binds HCMV gH is in a composition separate from the antibody which binds HCMV Complex I.

The invention also provides a method of treating, inhibiting or preventing HCMV infection comprising administering to a patient an effective amount of a composition comprising an anti-gH antibody, an anti-Complex I antibody or a combination thereof. The invention also provides for a method of treating, inhibiting or preventing congenital HCMV infection comprising administering to a pregnant woman an effective amount of a composition comprising an antibody of the invention or a combination thereof. The invention also provides a method of treating an HCMV infected fetus comprising administering to a pregnant woman an effective amount of a composition comprising an antibody of the invention or a combination thereof. The invention also provides a method of treating an HCMV infected infant, or infant exposed to HCMV during gestation, comprising administering to the infant an effective amount of a composition comprising an antibody of the invention or a combination thereof.

The invention also provides a method of treating, inhibiting or preventing HCMV infection in an organ or tissue transplant recipient comprising administering to the transplanted organ or tissue recipient an effective amount of composition comprising an antibody of the invention, or a combination thereof, to treat, inhibit or prevent HCMV infection arising from an organ or tissue which was obtained from an organ donor or tissue donor which is or has been infected with HCMV. Additional embodiments include methods in which the transplant recipient has previously been infected with HCMV and is at risk of reactivation. The method of treatment may further comprise administering an additional therapeutic agent to the patient (e.g., ganciclovir, foscarnet, valganciclovir and cidofovir).

In certain embodiments the composition comprising the antibody which binds HCMV gH is in a composition which is separate from the composition comprising the antibody which binds HCMV Complex I. In other embodiments the composition comprising the antibody which binds HCMV gH is administered simultaneously with, prior to or subsequent to the composition comprising the antibody which binds HCMV Complex I.

The invention also provides an anti-Complex I and/or an anti-gH antibody for use in inhibiting, treating or preventing HCMV infection. In some embodiments, the use is for inhibiting, treating or preventing congenital HCMV infection or HCMV infection in a tissue or organ transplant recipient for which the transplanted tissue, organ or the donor is or has been infected with HCMV. Additional embodiments include uses in which the transplant recipient has previously been infected with HCMV and is at risk of reactivation. In certain embodiments the tissue or organ transplant recipient is seronegative for HCMV infection.

The antibodies of the invention may be used in the manufacture of a medicament. The medicament may be for use in the treatment, inhibition or prevention of HCMV infection, such as, for example, inhibiting, preventing or treating congenital HCMV infection or HCMV infection in an organ or tissue transplant recipient for which the transplanted organ, tissue or the donor is or has been infected with HCMV. In additional embodiments the transplant recipient has previously been infected with HCMV and is at risk of reactivation. In certain embodiments, the medicament may further comprise an additional therapeutic agent (e.g., ganciclovir, foscarnet, valganciclovir and cidofovir). In certain embodiments the organ or tissue transplant recipient is seronegative for HCMV infection.

The invention also provides a method of treating, inhibiting or preventing HCMV infection comprising administering to a patient an effective amount of an anti-gH, anti-Complex I antibody or a combination thereof. The invention also provides for a method of treating, inhibiting or preventing congenital HCMV infection comprising administering to a pregnant woman an effective amount of an antibody of the invention or a combination thereof. The invention also provides a method of treating an HCMV infected fetus comprising administering to a pregnant woman an effective amount of an antibody of the invention or a combination thereof.

The invention also provides a method of treating, inhibiting or preventing HCMV infection in an organ or tissue transplant recipient comprising administering to the transplanted organ or tissue recipient an effective amount of an antibody of the invention, or a combination thereof, to treat, inhibit or prevent HCMV infection arising from an organ or tissue which was obtained from an organ donor or tissue donor which is or has been infected with HCMV. Additional embodiments include methods in which the transplant recipient has previously been infected with HCMV and is at risk of reactivation. The method of treatment may further comprise administering an additional therapeutic agent to the patient (e.g., ganciclovir, foscarnet, valganciclovir and cidofovir).

In certain embodiments, the antibody which binds HCMV gH is administered separately from the antibody which binds HCMV Complex I. In other embodiments, the antibody which binds HCMV gH is administered simultaneously with, prior to or subsequent to the antibody which binds HCMV Complex I.

In certain embodiments, the organ transplant is a heart, kidney, liver, lung, pancreas, intestine, or thymus. In other embodiments, the tissue transplant is hand, corneal, skin, face, islets of langerhans, bone marrow, stem cells, whole blood, platelets, serum, blood cells, blood vessels, heart valve, bone, bone progenitor cells, cartilage, ligaments, tendons, muscle lining.

The invention also provides for antibodies which bind to the same epitope as an anti-gH and/or an anti-Complex I antibody of the invention. Additional embodiments include antibodies which bind to an epitope of HCMV gH comprising amino acids which correspond to the amino acids selected from the group consisting of tryptophan at position 168 of SEQ ID NO: 1; aspartic acid at position 446 of SEQ ID NO:1; proline at position 171 of SEQ ID NO:1; and combinations thereof. Additional embodiments include antibodies which binds to an epitope of HCMV Complex I comprising amino acids which correspond to the amino acids selected from the group consisting of glutamine at position 47 of SEQ ID NO:203; (ii) lysine at position 51 of SEQ ID NO:203; (iii) aspartic acid at position 46 of SEQ ID NO:203; and (iv) combinations thereof. Additional embodiments include antibodies which bind to a polypeptide of HCMV Complex I, wherein the polypeptide comprises the amino acid sequence SRALPDQTRYKYVEQLVDLT LNYHYDAS (SEQ ID NO:194).

### BRIEF DESCRIPTION OF THE FIGURES

FIG. 1 shows an amino acid sequence alignment of the heavy chain variable region (VH) of murine mAb 8G8 (SEQ ID NO:50) with selected human heavy chain variable region: VH1 FW (SEQ ID NO:52), human VH3 FW (SEQ ID NO:53), and human VH7 FW (SEQ ID NO:54). The amino acids are numbered according to Kabat numbering. The hypervariable regions (HVRs) are boxed. Circles indicate VL-VH interactions (Padlan (1994) Mol. Immunol. 31:169); double asterisk (one over the other) indicates Vernier Positions (Foote and Winter (1992) J. Mol. Biol. 224:487) and FW-CDR interactions (Padlan (1994) Mol. Immunol. 31:169). Single asterisk at position 47, 64, 66, 68 indicates Vernier Positions (Foote and Winter (1992) J. Mol. Biol. 224:487); Single asterisk at position 58 indicates FW-CDR interaction (Padlan (1994) Mol. Immunol. 31:169).

FIG. 2 shows an amino acid sequence alignment of the light chain variable region (VL) of murine mAb 8G8 (SEQ ID NO:51) with human light chain variable region: λ3 FW region (SEQ ID NO:69) and human λ4 FW region (SEQ ID NO:55). The amino acids are numbered according to Kabat numbering. The hypervariable regions (HVRs) are boxed. Circles indicate VL-VH interactions (Padlan (1994) Mol. Immunol. 31:169); double asterisk (one over the other) indicates Vernier Positions (Foote and Winter (1992) J. Mol. Biol. 224:487) and FW-CDR interactions (Padlan (1994) Mol. Immunol. 31:169). Single asterisk at position 47, 64, 66, 68 indicates Vernier Positions (Foote and Winter (1992) J. Mol. Biol. 224:487); Single asterisk at position 58 indicates FW-CDR interaction (Padlan (1994) Mol. Immunol. 31:169).

FIG. 3 shows the results of a neutralization assay comparing 8G8λ3 variants with 8G8λ4 variants. Panel A: Humanized 8G8λ3 antibodies having a human VH1, VH3 or VH7 were used in neutralization assays beside a mouse/human chimeric 8G8 antibody (QE7/C2). Panel B: Humanized 8G8λ4 antibodies having a human VH1, VH3 or VH7 were used in neutralization assays beside a mouse/human chimeric 8G8 antibody (QE7/C2). EC50 values for the experiments appear below the respective experiments.

FIG. 4 shows mutant sequences in 8G8 HVR-L2. Shown are amino acid sequences of HVR-L2 and the first amino acid of FR3 (WT, SEQ ID NO:57; A1, SEQ ID NO:58; E1, SEQ ID NO:59; T1, SEQ ID NO:60; A2, SEQ ID NO:61; E2, SEQ ID NO:62; T2, SEQ ID NO:63; SG, SEQ ID NO:64; SGSG, SEQ ID NO:65; TGDA, SEQ ID NO:66). The numbers in the figure are based on Kabat numbering.

FIG. 5 shows the results of neutralization assays using the various humanized 8G8 antibodies with mutated HVR-L2 regions shown in FIG. 4 containing a single amino acid substitution. Panel A: Neutralization assay. The HVR-L2 mutant antibodies all contained a human 8G8 VH1 chain. Panel B: EC50 values for the experiment.

FIG. 6 shows results of neutralization assays using the various humanized 8G8 antibodies with mutated HVR-L2 regions shown in FIG. 4 containing two amino acid substitutions. Panel A: Neutralization assay. The HVR-L2 mutant antibodies all contained a human 8G8 VH1 chain. Panel B: EC50 values for the experiment.

FIG. 7 shows an amino acid sequence alignment of the light chain variable region of murine mAb 8G8 (SEQ ID NO:51) with human light chain variable region λ4 FW (SEQ ID NO:55) and humanized light chain variable region for 8G8 on λ4 FW (hu8G8.λ4 FW) (SEQ ID NO:48). The amino acids are numbered according to Kabat numbering. The hypervariable regions (HVRs) are boxed. Circles indicate VL-VH interactions (Padlan (1994) Mol. Immunol. 31:169); double asterisk (one over the other) indicates Vernier Positions (Foote and Winter (1992) J. Mol. Biol. 224:487) and FW-CDR interactions (Padlan (1994) Mol. Immunol. 31:169). Single asterisk at position 47, 64, 66, 68 indicates Vernier Positions (Foote and Winter (1992) J. Mol. Biol. 224:487); Single asterisk at position 58 indicates FW-CDR interaction (Padlan (1994) Mol. Immunol. 31:169).

FIG. 8 shows an amino acid sequence alignment of the heavy chain variable region of murine mAb 8G8 (SEQ ID NO:50) with human heavy chain variable region VH1 Framework (VH1 FW) (SEQ ID NO:52) and the humanized heavy chain variable region for 8G8 on VH1 FW (hu8G8.VH1) (SEQ ID NO:45). The amino acids are numbered according to Kabat numbering. The hypervariable regions (HVRs) are boxed. Circles indicate VL-VH interactions (Padlan (1994) Mol. Immunol. 31:169); double asterisk (one over the other) indicates Vernier Positions (Foote and Winter (1992) J. Mol. Biol. 224:487) and FW-CDR interactions (Padlan (1994) Mol. Immunol. 31:169). Single asterisk at position 47, 64, 66, 68 indicates Vernier Positions (Foote and Winter (1992) J. Mol. Biol. 224:487); Single asterisk at position 58 indicates FW-CDR interaction (Padlan (1994) Mol. Immunol. 31:169). An exemplary nucleic acid sequence encoding for hu8G8.VH1 is also shown (SEQ ID NO:185).

FIG. 9 shows an amino acid sequence alignment of the heavy chain variable region of murine mAb 8G8 (SEQ ID NO:50) with human heavy chain variable region VH3 FW (SEQ ID NO:53) and the humanized heavy chain variable region of 8G8 on VH3 FW (hu8G8.VH3) (SEQ ID NO:46). The amino acids are numbered according to Kabat numbering. The hypervariable regions (HVRs) are boxed. Circles indicate VL-VH interactions (Padlan (1994) Mol. Immunol. 31:169); double asterisk (one over the other) indicates Vernier Positions (Foote and Winter (1992) J. Mol. Biol. 224:487) and FW-CDR interactions (Padlan (1994) Mol. Immunol. 31:169). Single asterisk at position 47, 64, 66, 68 indicates Vernier Positions (Foote and Winter (1992) J. Mol. Biol. 224:487); Single asterisk at position 58 indicates FW-CDR interaction (Padlan (1994) Mol. Immunol. 31:169).

FIG. 10 shows an amino acid sequence alignment of the light chain variable region of murine mAb 8G8 V_{L} (SEQ ID NO:51) with the light chain variable region of λ4 FW region (SEQ ID NO:55) and the humanized light chain variable region of 8G8 on λ4 FW (λ4 8G8 graft) in which amino acid changes were introduced at amino acids 2 and 36 according to Kabat numbering (SEQ ID NO:49). The amino acids are numbered according to Kabat numbering. The hypervariable regions (HVRs) are boxed. Circles indicate VL-VH interactions (Padlan (1994) Mol. Immunol. 31:169); double asterisk (one over the other) indicates Vernier Positions (Foote and Winter (1992) J. Mol. Biol. 224:487) and FW-CDR interactions (Padlan (1994) Mol. Immunol. 31:169). Single asterisk at position 47, 64, 66, 68 indicates Vernier Positions (Foote and Winter (1992) J. Mol. Biol. 224:487); Single asterisk at position 58 indicates FW-CDR interaction (Padlan (1994) Mol. Immunol. 31:169). An exemplary nucleic acid sequence encoding for λ4 8G8 graft is also shown (SEQ ID NO:186).

FIG. 11 shows an amino acid sequence alignment of human antibody MSL-109 with mAb HB1. Panel A: An alignment of MSL-109 VL (SEQ ID NO:90) with affinity-matured HB1 VL (also SEQ ID NO:90 (100% identity)); and Panel B: an amino acid sequence alignment of human antibody MSL-109 VH (SEQ ID NO:92) with affinity-matured HB1 VH (SEQ ID NO:89). The amino acids are numbered according to Kabat numbering. The hypervariable regions (HVRs) are boxed.

FIG. 12A shows amino acid sequences of HVR-H2 from MSL-109 (SEQ ID NO:91) and IGHV3-21*01 (SEQ ID NO:93) and various amino acid substitutions made. FIG. 12B and FIG. 12C show the results of two different neutralization assays using the antibodies containing mutated HVR-H2 regions. Neutralization assays with the Fab (FIG. 12B) and the mAb (FIG. 12C) are both shown. The IC50s are provided in nM units.

FIG. 13 shows the results of a neutralization assay comparing an antibody containing λ4 8G8 graft and hu8G8.VH1 (hereinafter “hu8G8”) and HB1 with HIG for the ability to prevent infection of epithelial cells and fibroblasts.

FIG. 14 shows the results of a viral neutralization assay using depleted hyperimmune globulin (HIG) on epithelial cells and fibroblasts. HIG was depleted of anti-gB specific antibodies anti-Complex I specific antibodies, anti-gH/gL antibodies or mock-depletion as a control.

FIG. 15 shows the results of FACS analysis to determine the antigen specificity of HB1 and hu8G8 antibodies compared to a known anti-gB, anti-gH and anti-UL131 antibody. APC intensity on the x-axis indicates antibody binding. The y-axis plots the proportion of cells at a given intensity expressed as percentage of maximum number of cells at any intensity.

FIG. 16 shows the results of a neutralization assay in which hu8G8 and HB1 were mixed in a 1:1 ratio and tested in a dilution series for their ability to inhibit HCMV infection on epithelial cells. The combination of the two antibodies has additive effects and behave according to the Bliss independence equation (The combined response C for two single compounds with effects A and B is C=A+B−A*B).

FIG. 17 shows the results of a neutralization assay determining the potency of HB1 with varying concentrations of hu8G8 or hu8G8 with varying concentrations of HB1.

FIG. 18 shows the results of neutralization assays with HB1-resistant HCMV mutants. Panel A shows the results of a neutralization assay using the HB1 antibody. Panel B shows the results of a neutralization assay using the hu8G8 antibody. The HB1 resistant HCMV mutants are still sensitive to neutralization by hu8G8.

FIG. 19 shows the results of neutralization assays with hu8G8-resistant HCMV mutants. Panel A shows the results of a neutralization assay using the HB1 antibody. Panel B shows the results of a neutralization assay using the hu8G8 antibody. The hu8G8 resistant HCMV mutants are still sensitive to neutralization by HB1.

FIG. 20 shows data relating to viral entry of HCMV strain (WT) D1 (VR1814 grown in parallel when generating resistant strains) compared to the various HB1-resistant viral mutants on epithelial and fibroblast cells.

FIG. 21 shows the ability of HB1 antibody to bind to cell-surface expressed gH/gL containing resistance-conferring point mutations in gH, as assayed by FACS analysis. A different anti-gH antibody was used as a positive control for cell-surface expression. The x-axis is GFP intensity, which is an indicator of HCMV glycoprotein expression. The y-axis is APC signal, which indicates antibody binding.

FIG. 22 shows the ability of hu8G8 antibody to bind to cell-surface expressed Complex I containing resistance-conferring point mutations in Complex I, as assay by FACS analysis. An anti-UL131 antibody and an anti-gH antibody were used as positive controls for cell-surface expression. The x-axis is GFP intensity, which is an indicator of HCMV glycoprotein expression. The y-axis is APC signal, which indicates antibody binding.

FIGS. 23 A and B show the results of Scatchard analysis to determine the binding affinity of hu8G8 and HB1 for their antigen. Results were plotted using the fitting algorithm of Munson and Rodbard. The y-axis plots the ratio of the concentration of bound ¹²⁵I-labeled antibody to total antibody. Total antibody was calculated as the concentration of ¹²⁵I-labeled and unlabeled antibody.

FIG. 24 shows the results of an ELISA assay measuring the binding of hu8G8 and a positive control antibody (anti-HIS) to a peptide fragment (amino acid 41 (Ser) to amino acid 68 (Ser) of SEQ ID NO:194) of UL131 (SRA-Helix WT) or a corresponding fragment containing the amino acid substitution Q47K (SRA-Helix Mut).

### DETAILED DESCRIPTION OF EMBODIMENTS OF THE INVENTION

### I. Definitions

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The terms “anti-Complex I antibody” and “an antibody that binds to Complex I” refer to an antibody that is capable of binding Complex I with sufficient affinity such that the antibody is useful as a diagnostic and/or therapeutic agent in targeting Complex I. In one embodiment, the extent of binding of an anti-Complex I antibody to an unrelated, non-Complex I protein is less than about 10% of the binding of the antibody to Complex I as measured, e.g., by a radioimmunoassay (RIA). In certain embodiments, an antibody that binds to Complex I has a dissociation constant (Kd) of ≦1 μM, ≦100 nM, ≦10 nM, ≦1 nM, ≦0.1 nM, ≦0.01 nM, or ≦0.001 nM (e.g. 10⁻⁸M or less, e.g. from 10⁻⁸M to 10⁻¹³M, e.g., from 10⁻⁹M to 10⁻¹³ M). In certain embodiments, an anti-Complex I antibody binds to an epitope of Complex I that is conserved among human CMV isolates. In certain embodiments, an anti-Complex I antibody binds to an epitope of Complex I that is conserved among CMV strains that infect different species. In certain embodiments, the “anti-Complex I antibody” binds a conformational epitope of Complex I and in certain embodiments the anti-Complex I antibody binds to an epitope within an individual protein member of Complex I which is not gH (i.e., gL, UL128, UL130 or UL131).

The terms “anti-gH antibody” and “an antibody that binds to gH” refer to an antibody that is capable of binding gH with sufficient affinity such that the antibody is useful as a diagnostic and/or therapeutic agent in targeting gH. In one embodiment, the extent of binding of an anti-gH antibody to an unrelated, non-gH protein is less than about 10% of the binding of the antibody to gH as measured, e.g., by a radioimmunoassay (RIA). In certain embodiments, an antibody that binds to gH has a dissociation constant (Kd) of ≦1 μM, ≦100 nM, ≦10 nM, ≦1 nM, ≦0.1 nM, ≦0.01 nM, or ≦0.001 nM (e.g. 10⁻⁸M or less, e.g. from 10⁻⁸M to 10⁻¹³M, e.g., from 10⁻⁹M to 10⁻¹³ M). In certain embodiments, an anti-gH antibody binds to an epitope of gH that is conserved among human CMV isolates. In certain embodiments, an anti-gH antibody binds to an epitope of gH that is conserved among CMV strains that infect different species.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “Complex I,” as used herein, refers to any native Complex I from any cytomegalovirus source, including CMV that infects mammals such as primates (e.g., humans) and rodents (e.g., mice and rats), unless otherwise indicated. The term encompasses a combination of all of gH, gL, UL128, UL130 and UL131 polypeptides. The term also encompasses naturally occurring variants of the proteins of Complex I, e.g., splice variants or allelic variants. The amino acid sequence of an exemplary HCMV gH is shown in SEQ ID NO:1. The amino acid sequence of an exemplary HCMV gL is shown in SEQ ID NO:2. The amino acid sequence of an exemplary HCMV UL128 is shown in SEQ ID NO:3. The amino acid sequence of an exemplary HCMV UL130 is shown in SEQ ID NO:4. The amino acid sequence of an exemplary HCMV UL131 is shown in SEQ ID NO:5. Additional exemplary sequences for HCMV gH, gL, UL128, UL130 and UL131 may be found in Genbank Accession number GU179289 (Dargan et al., J. Gen. Virol. 91: 1535-1546 (2010)), which are both incorporated by reference herein in their entireties, and are included herein as SEQ ID NO: 206 (gH), SEQ ID NO: 208 (gL), SEQ ID NO: 205 (UL128), SEQ ID NO: 204 (UL130); and SEQ ID NO: 203 (UL131).

The term “Complex II,” as used herein, refers to any native Complex II from any cytomegalovirus source, including CMV that infects mammals such as primates (e.g., humans) and rodents (e.g., mice and rats), unless otherwise indicated. The term encompasses a combination of all of gH, gL and gO. The term also encompasses naturally occurring variants of the proteins of Complex II, e.g., splice variants or allelic variants. The amino acid sequence of an exemplary HCMV gH is shown in SEQ ID NO:1. The amino acid sequence of an exemplary HCMV gL is shown in SEQ ID NO:2. The amino acid sequence of an exemplary HCMV gO is shown in SEQ ID NO:209. Additional exemplary sequences for HCMV gH, gL and gO may be found in Genbank Accession number GU179289 (Dargan et al., J. Gen. Virol. 91: 1535-1546 (2010)), which are both incorporated by reference herein in their entireties, and are included herein as SEQ ID NO: 206 (gH), SEQ ID NO: 208 (gL) and SEQ ID NO: 207 (gO).

The term “gH,” as used herein, refers to any native gH from any vertebrate source, including mammals such as primates (e.g. humans) and rodents (e.g., mice and rats), unless otherwise indicated. The term encompasses “full-length,” unprocessed gH as well as any form of gH that results from processing in the cell. The term also encompasses naturally occurring variants of gH, e.g., splice variants or allelic variants. The amino acid sequence of gH is about 95% identical among CMV isolates. The amino acid sequence of an exemplary HCMV gH is shown in SEQ ID NO:1. An additional exemplary sequence for HCMV gH may be found in Genbank Accession number GU179289 (Dargan et al., J. Gen. Virol. 91: 1535-1546 (2010)), which are both incorporated by reference herein in their entireties, and is included herein as SEQ ID NO: 206 (gH).

The term “cytotoxic agent” as used herein refers to a substance that inhibits or prevents a cellular function and/or causes cell death or destruction. Cytotoxic agents include, but are not limited to radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

An “effective amount” of an agent, e.g., a pharmaceutical formulation, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH(H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, Md. (1991).) With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008).) Unless otherwise indicated, HVR residues and other residues in the variable domain (e.g., FR residues) are numbered herein according to Kabat et al., supra.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “infant” as used herein, refers to an individual or subject ranging in age from birth to not more than about one year and includes infants from 0 to about 12 months.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-Complex I antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

“Isolated nucleic acid encoding an anti-gH antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, compositions of the invention are used to delay development of a disease or to slow the progression of a disease or to decrease incidence of a disease or the severity of disease symptoms.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### II. Compositions and Methods

In one aspect, the invention is based, in part, on the discovery of monoclonal antibodies that neutralize infection of HCMV infection. In certain embodiments, antibodies that bind to Complex I are provided. In other embodiments, antibodies that bind to gH are provided. Antibodies of the invention are useful, e.g., for the prevention, inhibition and/or treatment of HCMV infection, congenital HCMV infection and infection of patients through HCMV-infected transplanted tissues. The antibodies may also be used for diagnosis of HCMV infection.

In one aspect, the invention is also based, in part, on the discovery of compositions comprising a combination of monoclonal antibodies which inhibit HCMV viral entry into all cell types of the placenta: endothelial cells, epithelial cells, monocytes/macrophages and fibroblasts and reduce and/or suppress the formation of HCMV resistant strains. In certain embodiments, methods of using these compositions are provided. The compositions of the invention are useful, e.g., for the prevention, inhibition and/or treatment of HCMV infection, congenital HCMV infection and infection of patients through HCMV-infected transplanted organs or tissues which have been harvested from patients previously or presently infected with HCMV. The compositions may also be used for the diagnosis of HCMV infection.

A. Exemplary Anti-Complex I Antibodies

In one aspect, the invention provides isolated antibodies that bind to Complex I. In certain embodiments, an anti-Complex I antibody specifically binds to a conformational epitope resulting from the association of UL128, UL130, UL131 with gH/gL or to an eptiope within an individual member of Complex I. In some embodiments, the anti-Complex I antibodies neutralize HCMV with an EC90 of 0.7 μg/ml, 0.5 μg/ml, 0.3 μg/ml, 0.1 μg/ml, 0.09 μg/ml, 0.08 μg/ml, 0.07 μg/ml, 0.06 μg/ml, 0.05 μg/ml, 0.04 μg/ml, 0.03 μg/ml, 0.02 μg/ml, 0.015, 0.012 μg/ml, 0.011 μg/ml, 0.010 μg/ml or less. In other aspects the anti-Complex I antibodies specifically bind to Complex I on the surface of HCMV and neutralize 50% of HCMV at an antibody concentration of 0.05 μg/ml, 0.02 μg/ml, 0.015 μg/ml, 0.014 μg/ml, 0.013 μg/ml, 0.012 μg/ml, 0.011 μg/ml, 0.010 μg/ml, 0.009 μg/ml, 0.008 μg/ml, 0.007 μg/ml, 0.006 μg/ml, 0.005 μg/ml, 0.004 μg/ml, 0.003 μg/ml, 0.002 μg/ml, 0.001 μg/ml, 0.0009 μg/ml, 0.0008 μg/ml, 0.0007 μg/ml or less (e.g., at an antibody concentration of 10⁻⁸M, 10⁻⁹M 10⁻¹⁰ M, 10⁻¹¹ M, 10⁻¹² M, 10⁻¹³M, or lower).

In one aspect, the invention provides an anti-Complex I antibody comprising at least one, two, three, four, five, or six HVRs selected from (a) HVR-H1 comprising the amino acid sequence of SEQ ID NO:6; (b) HVR-H2 comprising the amino acid sequence of SEQ ID NO:7; (c) HVR-H3 comprising the amino acid sequence of SEQ ID NO:8; (d) HVR-L1 comprising the amino acid sequence of SEQ ID NO:9; (e) HVR-L2 comprising an amino acid sequence selected from the group consisting of SEQ ID NOs:10-19; and (f) HVR-L3 comprising the amino acid sequence of SEQ ID NO:20.

In one aspect, the invention provides an antibody comprising at least one, at least two, or all three V_{H} HVR sequences selected from (a) HVR-H1 comprising the amino acid sequence of SEQ ID NO:6; (b) HVR-H2 comprising the amino acid sequence of SEQ ID NO:7; and (c) HVR-H3 comprising the amino acid sequence of SEQ ID NO:8.

In another aspect, the invention provides an antibody comprising at least one, at least two, or all three V_{L} HVR sequences selected from (a) HVR-L1 comprising the amino acid sequence of SEQ ID NO:9; (b) HVR-L2 comprising an amino acid sequence selected from SEQ ID NOs:10-19; and (c) HVR-L3 comprising the amino acid sequence of SEQ ID NO:20.

In one embodiment, the antibody comprises all three V_{H} HVR sequences selected from (a) HVR-H1 comprising the amino acid sequence of SEQ ID NO:6; (b) HVR-H2 comprising the amino acid sequence of SEQ ID NO:7; and (c) HVR-H3 comprising the amino acid sequence of SEQ ID NO:8 and three V_{L} HVR sequences selected from (a) HVR-L1 comprising the amino acid sequence of SEQ ID NO:9; (b) HVR-L2 comprising the amino acid sequence of SEQ ID NO:10; and (c) HVR-L3 comprising the amino acid sequence of SEQ ID NO:20.

In another embodiment, the antibody comprises all three V_{H} HVR sequences selected from (a) HVR-H1 comprising the amino acid sequence of SEQ ID NO:6; (b) HVR-H2 comprising the amino acid sequence of SEQ ID NO:7; and (c) HVR-H3 comprising the amino acid sequence of SEQ ID NO:8 and three V_{L} HVR sequences selected from (a) HVR-L1 comprising the amino acid sequence of SEQ ID NO:9; (b) HVR-L2 comprising the amino acid sequence of SEQ ID NO:11; and (c) HVR-L3 comprising the amino acid sequence of SEQ ID NO:20.

In another embodiment, the antibody comprises all three V_{H} HVR sequences selected from (a) HVR-H1 comprising the amino acid sequence of SEQ ID NO:6; (b) HVR-H2 comprising the amino acid sequence of SEQ ID NO:7; and (c) HVR-H3 comprising the amino acid sequence of SEQ ID NO:8 and three V_{L} HVR sequences selected from (a) HVR-L1 comprising the amino acid sequence of SEQ ID NO:9; (b) HVR-L2 comprising the amino acid sequence of SEQ ID NO:12; and (c) HVR-L3 comprising the amino acid sequence of SEQ ID NO:20.

In another embodiment, the antibody comprises all three V_{H} HVR sequences selected from (a) HVR-H1 comprising the amino acid sequence of SEQ ID NO:6; (b) HVR-H2 comprising the amino acid sequence of SEQ ID NO:7; and (c) HVR-H3 comprising the amino acid sequence of SEQ ID NO:8 and three V_{L} HVR sequences selected from (a) HVR-L1 comprising the amino acid sequence of SEQ ID NO:9; (b) HVR-L2 comprising the amino acid sequence of SEQ ID NO:13; and (c) HVR-L3 comprising the amino acid sequence of SEQ ID NO:20.

In another embodiment, the antibody comprises all three V_{H} HVR sequences selected from (a) HVR-H1 comprising the amino acid sequence of SEQ ID NO:6; (b) HVR-H2 comprising the amino acid sequence of SEQ ID NO:7; and (c) HVR-H3 comprising the amino acid sequence of SEQ ID NO:8 and three V_{L} HVR sequences selected from (a) HVR-L1 comprising the amino acid sequence of SEQ ID NO:9; (b) HVR-L2 comprising the amino acid sequence of SEQ ID NO:14; and (c) HVR-L3 comprising the amino acid sequence of SEQ ID NO:20.

In another embodiment, the antibody comprises all three V_{H} HVR sequences selected from (a) HVR-H1 comprising the amino acid sequence of SEQ ID NO:6; (b) HVR-H2 comprising the amino acid sequence of SEQ ID NO:7; and (c) HVR-H3 comprising the amino acid sequence of SEQ ID NO:8 and three V_{L} HVR sequences selected from (a) HVR-L1 comprising the amino acid sequence of SEQ ID NO:9; (b) HVR-L2 comprising the amino acid sequence of SEQ ID NO:15; and (c) HVR-L3 comprising the amino acid sequence of SEQ ID NO:20.

In another embodiment, the antibody comprises all three V_{H} HVR sequences selected from (a) HVR-H1 comprising the amino acid sequence of SEQ ID NO:6; (b) HVR-H2 comprising the amino acid sequence of SEQ ID NO:7; and (c) HVR-H3 comprising the amino acid sequence of SEQ ID NO:8 and three V_{L} HVR sequences selected from (a) HVR-L1 comprising the amino acid sequence of SEQ ID NO:9; (b) HVR-L2 comprising the amino acid sequence of SEQ ID NO:16; and (c) HVR-L3 comprising the amino acid sequence of SEQ ID NO:20.

In another embodiment, the antibody comprises all three V_{H} HVR sequences selected from (a) HVR-H1 comprising the amino acid sequence of SEQ ID NO:6; (b) HVR-H2 comprising the amino acid sequence of SEQ ID NO:7; and (c) HVR-H3 comprising the amino acid sequence of SEQ ID NO:8 and three V_{L} HVR sequences selected from (a) HVR-L1 comprising the amino acid sequence of SEQ ID NO:9; (b) HVR-L2 comprising the amino acid sequence of SEQ ID NO:17; and (c) HVR-L3 comprising the amino acid sequence of SEQ ID NO:20.

In another embodiment, the antibody comprises all three V_{H} HVR sequences selected from (a) HVR-H1 comprising the amino acid sequence of SEQ ID NO:6; (b) HVR-H2 comprising the amino acid sequence of SEQ ID NO:7; and (c) HVR-H3 comprising the amino acid sequence of SEQ ID NO:8 and three V_{L} HVR sequences selected from (a) HVR-L1 comprising the amino acid sequence of SEQ ID NO:9; (b) HVR-L2 comprising the amino acid sequence of SEQ ID NO:18; and (c) HVR-L3 comprising the amino acid sequence of SEQ ID NO:20.

In another embodiment, the antibody comprises all three V_{H} HVR sequences selected from (a) HVR-H1 comprising the amino acid sequence of SEQ ID NO:6; (b) HVR-H2 comprising the amino acid sequence of SEQ ID NO:7; and (c) HVR-H3 comprising the amino acid sequence of SEQ ID NO:8 and three V_{L} HVR sequences selected from (a) HVR-L1 comprising the amino acid sequence of SEQ ID NO:9; (b) HVR-L2 comprising the amino acid sequence of SEQ ID NO:19; and (c) HVR-L3 comprising the amino acid sequence of SEQ ID NO:20.

In some embodiments, the antibody comprises all three V_{H} HVR sequences selected from (a) HVR-H1 comprising the amino acid sequence of SEQ ID NO:6; (b) HVR-H2 comprising the amino acid sequence of SEQ ID NO:7; and (c) HVR-H3 comprising the amino acid sequence of SEQ ID NO:8 and three V_{L} HVR sequences selected from (a) HVR-L1 comprising the amino acid sequence of SEQ ID NO:9; (b) HVR-L2 and the first amino acid of the light chain variable region framework FR3 comprising the amino acid sequence of SEQ ID NO:21; and (c) HVR-L3 comprising the amino acid sequence of SEQ ID NO:20. In certain embodiments, any one or more amino acids of an anti-Complex I antibody as provided above are substituted at the following HVR positions: in HVR-L2 (SEQ ID NO:10): positions 4, 5, 11, and 12. In certain embodiments, the substitutions are conservative substitutions, as provided herein. In certain embodiments, any one or more of the following substitutions may be made in any combination: in HVR-L2 (SEQ ID NO:57): D4E, D4T, D4S, GSA, D11E, D11T, D11S, and G12A. All possible combinations of the above substitutions are encompassed by the consensus sequences of SEQ ID NO:21.

In any of the above embodiments, an anti-Complex I antibody is humanized. In one embodiment, an anti-Complex I antibody comprises HVRs as in any of the above embodiments, and further comprises an acceptor human framework, e.g. a human immunoglobulin framework or a human consensus framework. In another embodiment, an anti-Complex I antibody comprises HVRs as in any of the above embodiments, and further comprises a V_{H} comprising an FR1 sequence of SEQ ID NO:22, an FR2 sequence of SEQ ID NO:23, an FR3 sequence of SEQ ID NO:24, and an FR4 sequence of SEQ ID NO:25. In other embodiments, the anti-Complex I antibody comprises HVRs as in any of the above embodiments, and further comprises a V_{H} comprising an FR1 sequence of SEQ ID NO:22, a FR2 sequence of SEQ ID NO:27, a FR3 sequence of SEQ ID NO:28, and a FR4 sequence of SEQ ID NO:29. In other embodiments the anti-Complex I antibody comprises HVRs as in any of the above embodiments, and further comprises a V_{H} comprising an FR1 sequence of SEQ ID NO:30, a FR2 sequence of SEQ ID NO:31, a FR3 sequence of SEQ ID NO:32, and a FR4 sequence of SEQ ID NO:25. In other embodiments the anti-Complex I antibody comprises HVRs as in any of the above embodiments, and further comprises a V_{H} comprising an FR1 sequence of SEQ ID NO:33, a FR2 sequence of SEQ ID NO:23, a FR3 sequence of SEQ ID NO:34, and a FR4 sequence of SEQ ID NO:25.

In another embodiment, an anti-Complex I antibody comprises HVRs as in any of the above embodiments, and further comprises a V_{L} comprising an FR1 sequence of SEQ ID NO:35, an FR2 sequence of SEQ ID NO:36, an FR3 sequence of SEQ ID NO:37, and an FR4 sequence of SEQ ID NO:38. In other embodiments the anti-Complex I antibody comprises HVRs as in any of the above embodiments, and further comprises a V_{L} comprising an FR1 sequence of SEQ ID NO:39, a FR2 sequence of SEQ ID NO:40, a FR3 sequence of SEQ ID NO:41, and a FR4 sequence of SEQ ID NO:42. In other embodiments the anti-Complex I antibody comprises HVRs as in any of the above embodiments, and further comprises a V_{L} comprising an FR1 sequence of SEQ ID NO:43, a FR2 sequence of SEQ ID NO:44, a FR3 sequence of SEQ ID NO:41, and a FR4 sequence of SEQ ID NO:42.

In any of the above antibodies, the V_{L} FR3 sequence may be substituted with one selected from SEQ ID NO:67 or SEQ ID NO:68.

In another aspect, an anti-Complex I antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:46 or SEQ ID NO:47. In certain embodiments, a V_{H} sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-Complex I antibody comprising that sequence retains the ability to bind to Complex I. In certain embodiments, a total of 1 to 10 amino acids have been substituted, inserted and/or deleted in SEQ ID NO:45, or SEQ ID NO:46, or SEQ ID NO:47. In certain embodiments, substitutions, insertions, or deletions occur in regions outside the HVRs (i.e., in the FRs). In a particular embodiment, the V_{H} comprises one, two or three HVRs selected from: (a) HVR-H1 comprising the amino acid sequence of SEQ ID NO:6, (b) HVR-H2 comprising the amino acid sequence of SEQ ID NO:7, and (c) HVR-H3 comprising the amino acid sequence of SEQ ID NO:8.

In another aspect, an anti-Complex I antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:48 or SEQ ID NO:49. In certain embodiments, a V_{L} sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-Complex I antibody comprising that sequence retains the ability to bind to Complex I. In certain embodiments, a total of 1 to 10 amino acids have been substituted, inserted and/or deleted in SEQ ID NO:48 or SEQ ID NO:49. In certain embodiments, the substitutions, insertions, or deletions occur in regions outside the HVRs (i.e., in the FRs). In a particular embodiment, the V_{L} comprises one, two or three HVRs selected from (a) HVR-L1 comprising the amino acid sequence of SEQ ID NO:9; (b) HVR-L2 comprising the amino acid sequence selected from SEQ ID NOs:10-19; and (c) HVR-L3 comprising the amino acid sequence of SEQ ID NO:20.

In another aspect, an anti-Complex I antibody is provided, wherein the antibody comprises a V_{H} as in any of the embodiments provided above, and a V_{L} as in any of the embodiments provided above. In one embodiment, the antibody comprises the V_{H} and V_{L} sequences in SEQ ID NO:45 and SEQ ID NO:49, respectively, including post-translational modifications of those sequences. In one embodiment, the antibody comprises the V_{H} and V_{L} sequences in SEQ ID NO:46 and SEQ ID NO:49, respectively, including post-translational modifications of those sequences. In another embodiment, the antibody comprises the V_{H} and V_{L} sequences in SEQ ID NO:47 and SEQ ID NO:49, respectively, including post-translational modifications of those sequences. In another embodiment, the antibody comprises the V_{H} and V_{L} sequences in SEQ ID NO:45 and SEQ ID NO:48, respectively, including post-translational modifications of those sequences. In another embodiment, the antibody comprises the V_{H} and V_{L} sequences in SEQ ID NO:46 and SEQ ID NO:48, respectively, including post-translational modifications of those sequences. In another embodiment, the antibody comprises the V_{H} and V_{L} sequences in SEQ ID NO:47 and SEQ ID NO:48, respectively, including post-translational modifications of those sequences.

In a further aspect, the invention provides an antibody that competes with and/or binds to the same epitope as an anti-Complex I antibody provided herein. For example, in certain embodiments, an antibody is provided that competes with and/or binds to the same epitope as an anti-Complex I antibody comprising a V_{H} comprising an amino acid sequences of SEQ ID NOs:45-47 and a V_{L} comprising an amino acid sequence of SEQ ID NO:48 or SEQ ID NO:49.

In a further aspect, the invention provides an antibody that binds to the same epitope as an anti-Complex I antibody comprising amino acids which correspond to the amino acids selected from glutamine at amino acid position 47 of SEQ ID NO:203, lysine at amino acid position 51 of SEQ ID NO:203; aspartic acid at amino acid position 46 of SEQ ID NO:203 and combinations thereof. The corresponding amino acids which comprise the epitope may be at approximately the same location in the UL131 amino acid sequence but may differ due to amino acid sequence differences in UL131 between various HCMV strains.

In a further aspect, the invention provides an antibody that binds to a polypeptide of HCMV Complex I, wherein the polypeptide comprises the amino acid sequence SRALPDQTRYK YVEQLVDLTLNYHYDAS (SEQ ID NO:194).

In a further aspect, the invention provides an antibody that binds to the same epitope as an anti-Complex I antibody provided herein. In additional aspects, the invention provides an antibody that binds to the same epitope as an anti-Complex I antibody provided herein with an EC90 of 0.7 μg/ml, 0.5 μg/ml, 0.3 μg/ml, 0.1 μg/ml, 0.09 μg/ml, 0.08 μg/ml, 0.07 μg/ml, 0.06 μg/ml, 0.05 μg/ml, 0.04 μg/ml, 0.03 μg/ml, 0.02 μg/ml, 0.015, 0.012 μg/ml, 0.011 μg/ml, 0.010 μg/ml or less. In other aspects the invention provides an antibody that binds to the same epitope as an anti-Complex I antibody provided herein and which neutralizes 50% of HCMV at an antibody concentration of 0.05 μg/ml, 0.02 μg/ml, 0.015 μg/ml, 0.014 μg/ml, 0.013 μg/ml, 0.012 μg/ml, 0.011 μg/ml, 0.010 μg/ml, 0.009 μg/ml, 0.008 μg/ml, 0.007 μg/ml, 0.006 μg/ml, 0.005 μg/ml, 0.004 μg/ml, 0.003 μg/ml, 0.002 μg/ml, 0.001 μg/ml, 0.0009 μg/ml, 0.0008 μg/ml, 0.0007 μg/ml or less (e.g., at an antibody concentration of 10⁻⁸M, 10⁻⁹M 10⁻¹⁰ M, 10⁻¹¹ M, 10⁻¹² M, 10⁻¹³M, or lower).

In a further aspect of the invention, an anti-Complex I antibody according to any of the above embodiments is a monoclonal antibody, including a chimeric, humanized or human antibody. In one embodiment, an anti-Complex I antibody is an antibody fragment, e.g., a Fv, Fab, Fab′, scFv, diabody, or F(ab′)₂ fragment. In another embodiment, the antibody is a full length antibody, e.g., an intact IgG1 antibody or other antibody class or isotype as defined herein.

In a further aspect, an anti-Complex I antibody according to any of the above embodiments may incorporate any of the features, singly or in combination, as described in Sections 1-7 below.

B. Exemplary Anti-gH Antibodies

In one aspect, the invention provides isolated antibodies that bind to gH. In certain embodiments, an anti-gH antibody specifically binds an epitope of gH and neutralizes HCMV at an EC₉₀ of EC90 of 0.8 μg/ml, 0.7 μg/ml, 0.6 μg/ml, 0.5 μg/ml, 0.4 μg/ml, 0.3 μg/ml, 0.2 μg/ml, 0.1 μg/ml, 0.09 μg/ml, 0.08 μg/ml, 0.07 μg/ml, 0.06 μg/ml, 0.05 μg/ml, 0.04 μg/ml, 0.03 μg/ml, 0.02 μg/ml, 0.01 μg/ml, 0.015, 0.010 μg/ml or less. In some embodiments, the anti-gH antibodies specifically bind to an epitope of the gH/gL dimer produced in baculovirus with an IC50 in the range of 0.01 to 0.17 nM. In various embodiments, the IC50 may be 0.01 nM, 0.02 nM, 0.03 nM, 0.04 nM, 0.05 nM, 0.06 nM, 0.07 nM, 0.08 nM, 0.09 nM, 0.1 nM, 0.11 nM, 0.12 nM, 0.13 nM, 0.14 nM, 0.15 nM, 0.16 nM, or 0.17 nM.

In other embodiments, the antibodies bind to gH on the surface of HCMV and neutralize 50% of HCMV at an antibody concentration of 0.1 μg/ml, 0.09 μg/ml, 0.08 μg/ml, 0.07 μg/ml, 0.06 μg/ml, 0.05 μg/ml, 0.04 μg/ml, 0.03 μg/ml, 0.02 μg/ml, 0.015 μg/ml, 0.014 μg/ml, 0.013 μg/ml, 0.012 μg/ml, 0.011 μg/ml, 0.010 μg/ml, 0.009 μg/ml, 0.008 μg/ml, 0.007 μg/ml, 0.006 μg/ml, 0.005 μg/ml, 0.004 μg/ml, 0.003 μg/ml, 0.002 μg/ml, 0.001 μg/ml or less (e.g., at an antibody concentration of 10⁻⁸M, 10⁻⁹M 10⁻¹⁰ M, 10⁻¹¹ M, 10⁻¹² M, 10⁻¹³M, or lower).

In one aspect, the invention provides an anti-gH antibody comprising at least one, two, three, four, five, or six HVRs selected from (a) HVR-H1 comprising the amino acid sequence of SEQ ID NO:71; (b) HVR-H2 comprising an amino acid sequence selected from SEQ ID NO:72, 73 or 74; (c) HVR-H3 comprising the amino acid sequence of SEQ ID NO:75; (d) HVR-L1 comprising the amino acid sequence of SEQ ID NO:76; (e) HVR-L2 comprising the amino acid sequence of SEQ ID NO:77; and (f) HVR-L3 comprising the amino acid sequence of SEQ ID NO:78.

In one embodiment, the invention provides an antibody comprising at least one, at least two, or all three VH HVR sequences selected from (a) HVR-H1 comprising the amino acid sequence of SEQ ID NO:71; (b) HVR-H2 comprising the amino acid sequence of SEQ ID NO:72; and (c) HVR-H3 comprising the amino acid sequence of SEQ ID NO:75. In another embodiment, the antibody comprises at least one, at least two, or all three VH HVR sequences selected from (a) HVR-H1 comprising the amino acid sequence of SEQ ID NO:71; (b) HVR-H2 comprising the amino acid sequence of SEQ ID NO:73; and (c) HVR-H3 comprising the amino acid sequence of SEQ ID NO:75. In another embodiment, the antibody comprises at least one, at least two, or all three VH HVR sequences selected from (a) HVR-H1 comprising the amino acid sequence of SEQ ID NO:71; (b) HVR-H2 comprising the amino acid sequence of SEQ ID NO:74; and (c) HVR-H3 comprising the amino acid sequence of SEQ ID NO:75.

In another aspect, the invention provides an antibody comprising at least one, at least two, or all three VL HVR sequences selected from (a) HVR-L1 comprising the amino acid sequence of SEQ ID NO:76; (b) HVR-L2 comprising the amino acid sequence of SEQ ID NO:77; and (c) HVR-L3 comprising the amino acid sequence of SEQ ID NO:78 and an HVR-H2 comprising an amino acid sequence selected from SEQ ID NO:72, SEQ ID NO:73, or SEQ ID NO:74.

In another aspect, the invention provides an antibody comprising (a) a VH domain comprising at least one, at least two, or all three VH HVR sequences selected from (i) HVR-H1 comprising the amino acid sequence of SEQ ID NO:71, (ii) HVR-H2 comprising an amino acid sequence selected from SEQ ID NO:72, SEQ ID NO:73, or SEQ ID NO:74, and (iii) HVR-H3 comprising an amino acid sequence selected from SEQ ID NO:75; and (b) a VL domain comprising at least one, at least two, or all three VL HVR sequences selected from (i) HVR-L1 comprising the amino acid sequence of SEQ ID NO:76, (ii) HVR-L2 comprising the amino acid sequence of SEQ ID NO:77, and (c) HVR-L3 comprising the amino acid sequence of SEQ ID NO:78.

In another aspect, the invention provides an antibody comprising (a) HVR-H1 comprising the amino acid sequence of SEQ ID NO:71; (b) HVR-H2 comprising the amino acid sequence of SEQ ID NO:72; (c) HVR-H3 comprising the amino acid sequence of SEQ ID NO:75; (d) HVR-L1 comprising the amino acid sequence of SEQ ID NO:76; (e) HVR-L2 comprising the amino acid sequence of SEQ ID NO:77; and (f) HVR-L3 comprising an amino acid sequence selected from SEQ ID NO:78.

In another aspect, the invention provides an antibody comprising (a) HVR-H1 comprising the amino acid sequence of SEQ ID NO:71; (b) HVR-H2 comprising the amino acid sequence of SEQ ID NO:73; (c) HVR-H3 comprising the amino acid sequence of SEQ ID NO:75; (d) HVR-L1 comprising the amino acid sequence of SEQ ID NO:76; (e) HVR-L2 comprising the amino acid sequence of SEQ ID NO:77; and (f) HVR-L3 comprising an amino acid sequence selected from SEQ ID NO:78.

In another aspect, the invention provides an antibody comprising (a) HVR-H1 comprising the amino acid sequence of SEQ ID NO:71; (b) HVR-H2 comprising the amino acid sequence of SEQ ID NO:74; (c) HVR-H3 comprising the amino acid sequence of SEQ ID NO:75; (d) HVR-L1 comprising the amino acid sequence of SEQ ID NO:76; (e) HVR-L2 comprising the amino acid sequence of SEQ ID NO:77; and (f) HVR-L3 comprising an amino acid sequence selected from SEQ ID NO:78.

In certain embodiments, any one or more amino acids of an anti-gH antibody as provided above are substituted at the following HVR positions: in HVR-H2 (SEQ ID NO:91): positions 6 and 8. In certain embodiments, the substitutions are conservative substitutions, as provided herein. In certain embodiments, any one or more of the following substitutions may be made in any combination: in HVR-H2 (SEQ ID NO:91): D6S, D6T, D6N, D6Q, D6F, D6M, D6L, and T8R. All possible combinations of the above substitutions are encompassed by the consensus sequences of SEQ ID NO:93.

In any of the above embodiments, an anti-gH antibody is humanized. In one embodiment, an anti-gH antibody comprises HVRs as in any of the above embodiments, and further comprises an acceptor human framework, e.g. a human immunoglobulin framework or a human consensus framework. In another embodiment, an anti-gH antibody comprises HVRs as in any of the above embodiments, and further comprises a V_{H} comprising an FR1 sequence of SEQ ID NO:79, an FR2 sequence of SEQ ID NO:80, an FR3 sequence of SEQ ID NO:81, and an FR4 sequence of SEQ ID NO:82. In other embodiments, the anti-gH antibody comprises HVRs as in any of the above embodiments, and further comprises a V_{L} comprising an FR1 sequence of SEQ ID NO:83, a FR2 sequence of SEQ ID NO:84, a FR3 sequence of SEQ ID NO:85, and a FR4 sequence of SEQ ID NO:86.

In another aspect, an anti-gH antibody of the invention comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:92 but wherein the amino acid at position 54 is Asn (N) and/or wherein the amino acid at position 56 is Asn (R). In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-gH antibody comprising that sequence retains the ability to bind to gH. In certain embodiments, a total of 1 to 10 amino acids have been substituted, inserted and/or deleted in SEQ ID NO:92 but wherein the amino acid at position 54 is Asn (N) and/or wherein the amino acid at position 56 is Asn (R). In certain embodiments, substitutions, insertions, or deletions occur in regions outside the HVRs (i.e., in the FRs). Optionally, the anti-gH antibody comprises the VH sequence in SEQ ID NO: 87, SEQ ID NO:88 or SEQ ID NO:89, including post-translational modifications of that sequence. In a particular embodiment, the VH comprises one, two or three HVRs selected from: (a) HVR-H1 comprising the amino acid sequence of SEQ ID NO:71, (b) HVR-H2 comprising an amino acid sequence selected from SEQ ID NO:72, SEQ ID NO:73 and SEQ ID NO:74, and (c) HVR-H3 comprising the amino acid sequence of SEQ ID NO:75.

In another aspect, an anti-gH antibody of the invention comprises a light chain variable domain (VL) having at least 90%, 91%, 92%, 93%, 94%, 95%, 96%, 97%, 98%, 99%, or 100% sequence identity to the amino acid sequence of SEQ ID NO:90. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-gH antibody comprising that sequence retains the ability to bind to gH. In certain embodiments, a total of 1 to 10 amino acids have been substituted, inserted and/or deleted in SEQ ID NO:90. In certain embodiments, the substitutions, insertions, or deletions occur in regions outside the HVRs (i.e., in the FRs). Optionally, the anti-gH antibody comprises the VL sequence in SEQ ID NO:90, including post-translational modifications of that sequence. In a particular embodiment, the VL comprises one, two or three HVRs selected from (a) HVR-L1 comprising the amino acid sequence of SEQ ID NO:76; (b) HVR-L2 comprising the amino acid sequence of SEQ ID NO:77; and (c) HVR-L3 comprising the amino acid sequence of SEQ ID NO:78.

In another aspect, an anti-gH antibody of the invention comprises a VH as in any of the embodiments provided above, and a VL as in any of the embodiments provided above. In one embodiment, the antibody comprises the VH and VL sequences in SEQ ID NO:87 and SEQ ID NO:90, respectively, including post-translational modifications of those sequences.

In another embodiment, the antibody comprises the VH and VL sequences in SEQ ID NO:88 and SEQ ID NO:90, respectively, including post-translational modifications of those sequences.

In another embodiment, the antibody comprises the VH and VL sequences in SEQ ID NO:89 and SEQ ID NO:90, respectively, including post-translational modifications of those sequences.

In a further aspect, the invention provides an antibody that competes with and/or binds to the same epitope as an anti-gH antibody provided herein. For example, in certain embodiments, an antibody is provided that competes with and/or binds to the same epitope as an anti-gH antibody comprising a V_{H} comprising an amino acid sequences of SEQ ID NOs:87, 88 or 89 and a V_{L} comprising an amino acid sequence of SEQ ID NO:90.

In a further aspect, the invention provides an antibody that binds to the same epitope as an anti-gH antibody comprising amino acids which correspond to the amino acids selected from tryptophan at amino acid position 168 of SEQ ID NO:1, aspartic acid at amino acid position 446 of SEQ ID NO:1; proline at amino acid position 171 of SEQ ID NO:1 and combinations thereof. The corresponding amino acids which comprise the epitope may be at approximately the same location in the gH amino acid sequence but may differ due to amino acid sequence differences in gH between various HCMV strains.

In additional aspects, the invention provides an antibody that binds to the same epitope as an anti-gH antibody provided herein with an IC₅₀ in the range of 0.01 to 0.17 nM. In various embodiments, the IC₅₀ may be 0.17 nM or less (e.g., 0.16 nM, 0.15 nM, 0.14 nM, 0.13 nM, 0.12 nM, 0.11 nM, 0.10 nM, 0.09 nM, 0.08 nM, 0.07 nM, 0.06 nM, 0.05 nM, 0.04 nM, 0.03 nM, 0.02 nM, 0.01 nM or less. For example, in certain embodiments, an antibody is provided that binds to the same epitope as HB1 (an anti-gH antibody comprising a VH sequence of SEQ ID NO:89 and a VL sequence of SEQ ID NO:90) and has an IC₅₀ of 0.17 nM or less (e.g., 0.16 nM, 0.15 nM, 0.14 nM, 0.13 nM, 0.12 nM, 0.11 nM, 0.10 nM, 0.09 nM, 0.08 nM, 0.07 nM, 0.06 nM, 0.05 nM, 0.04 nM, 0.03 nM, 0.02 nM, 0.01 nM or less), or neutralizes HCMV infection at an EC90 of 0.8 μg/ml, 0.7 μg/ml, 0.6 μg/ml, 0.5 μg/ml, 0.4 μg/ml, 0.3 μg/ml, 0.2 μg/ml, 0.1 μg/ml, 0.09 μg/ml, 0.08 μg/ml, 0.07 μg/ml, 0.06 μg/ml, 0.05 μg/ml, 0.04 μg/ml, 0.03 μg/ml, 0.02 μg/ml, 0.01 μg/ml, 0.015, 0.010 μg/ml or less.

In other aspects, the invention provides an antibody that binds to the same epitope as an anti-gH antibody provided herein and neutralize 50% of HCMV at an antibody concentration of 0.1 μg/ml, 0.09 μg/ml, 0.08 μg/ml, 0.07 μg/ml, 0.06 μg/ml, 0.05 μg/ml, 0.04 μg/ml, 0.03 μg/ml, 0.02 μg/ml, 0.015 μg/ml, 0.014 μg/ml, 0.013 μg/ml, 0.012 μg/ml, 0.011 μg/ml, 0.010 μg/ml, 0.009 μg/ml, 0.008 μg/ml, 0.007 μg/ml, 0.006 μg/ml, 0.005 μg/ml, 0.004 μg/ml, 0.003 μg/ml, 0.002 μg/ml, 0.001 μg/ml or less (e.g., at an antibody concentration of 10⁻⁸M, 10⁻⁹M 10⁻¹⁰ M, 10⁻¹¹ M, 10⁻¹² M, 10⁻¹³M, or lower).

In a further aspect of the invention, an anti-gH antibody according to any of the above embodiments is a monoclonal antibody, including a chimeric, humanized or human antibody. In one embodiment, an anti-gH antibody is an antibody fragment, e.g., a Fv, Fab, Fab′, scFv, diabody, or F(ab′)₂ fragment. In another embodiment, the antibody is a full length antibody, e.g., an intact IgG1 antibody or other antibody class or isotype as defined herein.

In a further aspect, an anti-gH antibody according to the above embodiments may incorporate any of the features, singly or in combination, as described in Sections 1-7 below.

1. Antibody Affinity

In certain embodiments, an antibody of the invention, as provided herein, has a dissociation constant (Kd) of ≦1 μM, ≦100 nM, ≦10 nM, ≦1 nM, ≦0.1 nM, ≦0.01 nM, or ≦0.001 nM (e.g. 10⁻⁸M or less, e.g. from 10⁻⁸M to 10⁻¹³M, e.g., from 10⁻⁹M to 10⁻¹³ M).

In one embodiment, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE®-2000 or a BIACORE®-3000 (BIAcore, Inc., Piscataway, N.J.) at 25° C. with immobilized antigen CM5 chips at ˜10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

2. Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

3. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

4. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26 (4):265-268 (2006) (describing human-human hybridomas). Human hybridoma technology (Trioma technology) is also described in Vollmers and Brandlein, Histology and Histopathology, 20 (3):927-937 (2005) and Vollmers and Brandlein, Methods and Findings in Experimental and Clinical Pharmacology, 27 (3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

Antibodies in the compositions of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338 (2): 299-310 (2004); Lee et al., J. Mol. Biol. 340 (5): 1073-1093 (2004); Fellouse, Proc. Natl. Acad. Sci. USA 101 (34): 12467-12472 (2004); and Lee et al., J. Immunol. Methods 284 (1-2): 119-132 (2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

6. Multispecific Antibodies

In certain embodiments, an antibody provided herein is a multispecific antibody, e.g. a bispecific antibody. Multispecific antibodies are monoclonal antibodies that have binding specificities for at least two different sites. In certain embodiments, one of the binding specificities is for Complex I or gH and the other is for any other antigen. In certain embodiments, one of the binding specificities is for Complex I and the other is for gH. In certain embodiments, bispecific antibodies may bind to two different epitopes of Complex I or gH. Bispecific antibodies may also be used to localize cytotoxic agents to cells which have Complex I or gH on the cell surface. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148 (5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to Complex I or gH as well as another, different antigen (see, US 2008/0069820, for example).

7. Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

a) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 1 under the heading of “conservative substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

b) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94 (4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

c) Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18 (12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9 (2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. No. 5,648,260; U.S. Pat. No. 5,624,821; and WO 94/29351 concerning other examples of Fc region variants.

d) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

e) Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

C. Recombinant Methods and Compositions

Antibodies may be produced using recombinant methods and compositions, e.g., as described in U.S. Pat. No. 4,816,567. In one embodiment, isolated nucleic acid encoding an anti-Complex I antibody or an anti-gH antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-Complex I antibody or anti-gH antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-Complex I antibody or an anti-gH antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TR1 cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

D. Assays

Anti-Complex I antibodies or anti-gH antibodies provided herein may be identified, screened for, or characterized for their physical/chemical properties and/or biological activities by various assays known in the art.

1. Binding Assays and Other Assays

In one aspect, an antibody of the invention is tested for its antigen binding activity, e.g., by known methods such as ELISA, Western blot, etc.

In another aspect, competition assays may be used to identify an antibody that competes for binding of Complex I with anti-Complex I antibodies described herein.

In another aspect, competition assays may be used to identify an antibody that competes for binding of gH with anti-gH antibodies described herein.

In certain embodiments, such a competing antibody binds to the same epitope (e.g., a linear or a conformational epitope) of gH or Complex I.

Detailed exemplary methods for mapping an epitope to which an antibody binds are provided in Morris (1996) “Epitope Mapping Protocols,” in Methods in Molecular Biology vol. 66 (Humana Press, Totowa, N.J.).

In an exemplary competition assay, immobilized Complex I or gH is incubated in a solution comprising a first labeled antibody that binds to Complex I or gH, respectively and a second unlabeled antibody that is being tested for its ability to compete with the first antibody for binding to Complex I or gH. The second antibody may be present in a hybridoma supernatant. As a control, immobilized Complex I or gH is incubated in a solution comprising the first labeled antibody but not the second unlabeled antibody. After incubation under conditions permissive for binding of the first antibody to Complex I or gH, excess unbound antibody is removed, and the amount of label associated with immobilized Complex I or gH is measured. If the amount of label associated with immobilized Complex I or gH is substantially reduced in the test sample relative to the control sample, then that indicates that the second antibody is competing with the first antibody for binding to Complex I or gH. See Harlow and Lane (1988) Antibodies: A Laboratory Manual ch. 14 (Cold Spring Harbor Laboratory, Cold Spring Harbor, N.Y.).

Competition assays can also be performed in a manner as described above with FACS using cells transfected with gH and/or other members of Complex I or Complex II and expressed on the cell surface. Additionally, ELISA with gH and/or reconstituted Complex I or Complex II can also be used in a competition assay. The use of FACS and ELISA to measure anti-gH and anti-Complex I antibodies is further described in the Examples.

2. Activity Assays

In one aspect, assays are provided for identifying anti-Complex I antibodies thereof having biological activity. Biological activity may include, e.g., specifically binding to a conformational epitope resulting from the association of UL128, UL130, UL131 and gH/gL, or specifically binding to an epitope within a single protein of Complex I, neutralizing HCMV at an EC₉₀ of 0.7 μg/ml or less. In some embodiments, the EC₉₀ is 0.5 μg/ml or less. In other embodiments the EC₉₀ is 0.3 μg/ml or less. In other embodiments the EC₉₀ is 0.1 μg/ml or less. In other embodiments the EC₉₀ is 0.08 μg/ml or less. In other embodiments the EC₉₀ is 0.06 μg/ml or less. In still other embodiments the EC₉₀ is 0.04 μg/ml or less. In other embodiments the EC₉₀ is 0.02 μg/ml or less. In other embodiments the EC₉₀ is 0.015 μg/ml or less. In other embodiments the EC₉₀ is 0.012 μg/ml or less. In other embodiments the EC₉₀ is 0.011 μg/ml or less. In other embodiments the EC₉₀ is 0.010 μg/ml or less. Compositions comprising antibodies having such biological activity are also provided.

In one aspect, assays are provided for identifying anti-gH antibodies thereof having biological activity. Biological activity may include, e.g., neutralization of HCMV at an EC90 of EC90 of 1 μg/ml, 0.9 μg/ml, 0.8 μg/ml, 0.7 μg/ml, 0.6 μg/ml, 0.5 μg/ml, 0.4 μg/ml, 0.3 μg/ml, 0.2 μg/ml, 0.1 μg/ml, 0.09 μg/ml, 0.08 μg/ml, 0.07 μg/ml, 0.06 μg/ml, 0.05 μg/ml, 0.04 μg/ml or less.

Anti-gH antibodies in the compositions of the invention bind to a gH/gL dimer expressed in baculovirus with an IC50 of 0.17 nM or less (e.g., 0.16 nM, 0.15 nM, 0.14 nM, 0.13 nM, 0.12 nM, 0.11 nM, 0.10 nM, 0.09 nM, 0.08 nM, 0.07 nM, 0.06 nM, 0.05 nM, 0.04 nM, 0.03 nM, 0.02 nM, 0.01 nM or less. Compositions comprising antibodies having such biological activity in vivo and/or in vitro are also provided.

In certain embodiments, an antibody of the invention is tested for such biological activity. See Example 3 for an exemplary description of such an assay.

E. Immunoconjugates

The invention also provides compositions comprising immunoconjugates comprising an anti-Complex I antibody or an anti-gH antibody herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

In another embodiment, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio)propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

F. Methods and Compositions for Diagnostics and Detection

In certain embodiments, any of the anti-Complex I antibodies and/or anti-gH antibodies, or compositions comprising such antibodies, as provided herein, are useful for detecting the presence of Complex I and/or gH in a biological sample. The term “detecting” as used herein encompasses quantitative or qualitative detection. In certain embodiments, a biological sample comprises a cell or tissue, such as placenta, kidney, heart, lung, liver, pancreas, intestine, thymus, bone, tendon, cornea, skin, heart valves, and veins. Furthermore, compositions comprising the antibodies may be used to detect HCMV in endothelial cells, epithelial cells, fibroblasts and macrophages.

In one embodiment, an anti-Complex I antibody and/or an anti-gH antibody for use in a method of diagnosis or detection is provided. In a further aspect, a method of detecting the presence of Complex I and/or gH in a biological sample is provided. In certain embodiments, the method comprises contacting the biological sample with an anti-Complex I antibody and/or an anti-gH antibody, as described herein, under conditions permissive for binding of the anti-Complex I antibody to Complex I and/or the binding of the anti-gH antibody to gH, and detecting whether a complex is formed between the anti-Complex I antibody and Complex I and/or the anti-gH antibody and gH. Such method may be an in vitro or in vivo method. In one embodiment, an anti-Complex I antibody or an anti-gH antibody or a combination of an anti-Complex I antibody and an anti-gH antibody is used to select subjects eligible for therapy with a anti-Complex I antibody or an anti-gH antibody or a combination of an anti-Complex I antibody and an anti-gH antibody, e.g. where Complex I and gH is a biomarker for selection of patients.

Exemplary disorders that may be diagnosed using a composition of the invention include HCMV infection, such as HCMV infection from transplanted organs or tissues, congenital HCMV infection, HCMV infection during pregnancy, and HCMV infection in children, infants and adults.

In certain embodiments, compositions comprising labeled anti-Complex I antibodies and/or anti-gH antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

G. Pharmaceutical Formulations

Pharmaceutical formulations of an anti-Complex I antibody or an anti-gH antibody or a combination of an anti-Complex I antibody and an anti-gH antibody, as described herein, are prepared by mixing such antibodies having the desired degree of purity with one or more optional pharmaceutically acceptable carriers (Remington's Pharmaceutical Sciences 16th edition, Osol, A. Ed. (1980)), in the form of lyophilized formulations or aqueous solutions. As described herein, anti-Complex I antibody and anti-gH antibody may be formulated in a single combined pharmaceutical formulation or in separate pharmaceutical formulations. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain active ingredients, in addition to the anti-Complex I antibody and/or the anti-gH antibody, as necessary for the particular indication being treated, preferably those with complementary activities that do not adversely affect each other. For example, it may be desirable to further provide ganciclovir, foscarnet, valganciclovir and cidofovir. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

H. Therapeutic Methods and Compositions

Any of the compositions comprising the anti-Complex I antibodies and/or anti-gH antibodies provided herein may be used in therapeutic methods.

In one aspect, compositions comprising an anti-Complex I antibody or an anti-gH antibody or an anti-Complex I antibody and an anti-gH antibody for use as a medicament is provided. In further aspects, compositions comprising an anti-Complex I antibody or an anti-gH antibody or an anti-Complex I antibody and an anti-gH antibody for use in treating HCMV infection is provided. In certain embodiments, compositions comprising an anti-Complex I antibody or an anti-gH antibody or an anti-Complex I antibody and an anti-gH antibody for use in a method of treatment is provided. In certain embodiments, the invention provides compositions comprising an anti-Complex I antibody or an anti-gH antibody or an anti-Complex I antibody and an anti-gH antibody for use in a method of treating an individual having an HCMV infection comprising administering to the individual an effective amount of the composition comprising an anti-Complex I antibody and/or an anti-gH antibody. In other embodiments the invention provides compositions for use in a method of preventing, inhibiting or treating congenital HCMV infection or HCMV infection in a tissue or organ transplant recipient for which the transplanted tissue, organ or donor is or has been infected with HCMV. In one such embodiment, the tissue or organ transplant recipient in seronegative for HCMV infection. In additional embodiments, the transplant recipient or individual has previously been infected with HCMV and is at risk of HCMV reactivation and infection. In certain embodiments, the method further comprises administering to the individual or transplant recipient an effective amount of at least one additional therapeutic agent, e.g., as described below. In other embodiments, the invention also provides compositions comprising an anti-Complex I antibody or an anti-gH antibody or an anti-Complex I antibody and an anti-gH antibody for use in a method or treatment of an HCMV infected infant, or infant exposed to HCMV during gestation, comprising administering to the infant an effective amount of a composition comprising an antibody of the invention or a combination thereof. In further embodiments, the invention provides compositions comprising an anti-Complex I antibody or an anti-gH antibody or an anti-Complex I antibody and an anti-gH antibody for use in treating, inhibiting or preventing HCMV infection in an individual at risk for infection. An “individual” according to any of the above embodiments is preferably a human.

In a further aspect, the invention provides for the use of a composition comprising an anti-Complex I antibody and/or an anti-gH antibody or an anti-Complex I antibody and an anti-gH antibody in the manufacture or preparation of a medicament. In one embodiment, the medicament is for treating, preventing or inhibition HCMV infection. In a further embodiment, the medicament is for use in treating, preventing or inhibiting HCMV infection comprising administering to an individual having an HCMV infection an effective amount of the medicament. In other embodiments the medicament is for use in a method of preventing, inhibiting or treating congenital HCMV infection or HCMV infection in a tissue or organ transplant recipient for which the transplanted tissue, organ or donor is or has been infected with HCMV. In one such embodiment, the tissue or organ transplant recipient in seronegative for HCMV infection. In additional embodiments, the transplant recipient or individual has previously been infected with HCMV and is at risk of HCMV reactivation and infection. In certain embodiments, the medicament further comprises an effective amount of at least one additional therapeutic agent, e.g., as described below. In a further embodiment, the medicament is for use in treating, inhibiting or preventing an HCMV infection in an individual at risk for infection comprising administering to the individual an amount effective of the medicament to inhibit or prevent HCMV infection. In other embodiments, the medicament is for use in treating an HCMV infected infant, or infant exposed to HCMV during gestation, comprising administering to the infant an effective amount of a composition comprising an antibody of the invention or a combination thereof. An “individual” according to any of the above embodiments may be a human. In certain embodiments, the medicament is for reducing HCMV viral titer or preventing an increase in HCMV viral titer in an individual. In one embodiment, the method comprises administering to the individual an effective amount of a composition comprising an anti-Complex I antibody and/or an anti-gH antibody to reduce HCMV viral titer or prevent an increase in HCMV viral titer. In one embodiment, an “individual” is a human, and/or pregnant and/or an organ transplant recipient at risk for HCMV infection.

In a further aspect, the invention provides a method for treating, preventing or inhibiting an HCMV infection. In one embodiment, the method comprises administering to an individual an effective amount of a composition comprising an anti-Complex I antibody and/or an anti-gH antibody. In other embodiments the invention provides a method of preventing, inhibiting or treating congenital HCMV infection or HCMV infection in a tissue or organ transplant recipient, for which the transplanted tissue, organ or donor is or has been infected with HCMV, comprising administering to an individual or transplant recipient an effective amount of a composition comprising an anti-Complex I antibody and an anti-gH antibody. In one such embodiment, the tissue or organ transplant recipient in seronegative for HCMV infection. In additional embodiments, the transplant recipient or individual has previously been infected with HCMV and is at risk of HCMV reactivation and infection. In one such embodiment, the method further comprises administering to the individual an effective amount of at least one additional therapeutic agent, as described below. In other embodiments, the invention provides a method for treating, inhibiting or preventing an HCMV infected infant, or infant exposed to HCMV during gestation, comprising administering to the infant an effective amount of a composition comprising an antibody of the invention or a combination thereof. An “individual” according to any of the above embodiments may be a human.

In a further aspect, the invention provides a method for inhibiting or preventing an HCMV infection in an individual at risk for infection. In one embodiment, the method comprises administering to the individual an effective amount of a composition comprising an anti-Complex I antibody and/or an anti-gH antibody to inhibit or prevent HCMV infection. In one embodiment, an “individual” is a human.

In certain embodiments, the invention provides a method for reducing HCMV viral titer or preventing an increase in HCMV viral titer in an individual. In one embodiment, the method comprises administering to the individual an effective amount of a composition comprising an anti-Complex I antibody and/or an anti-gH antibody to reduce HCMV viral titer or prevent an increase in HCMV viral titer. In one embodiment, an “individual” is a human, and/or pregnant and/or an organ transplant recipient at risk for HCMV infection.

HCMV viral titer can be measured by any means know in the art, for example by ELISA to measure viral antibodies, serological or tissue based assays to measure the presence of HCMV by quantifying the amount of viral DNA (either specific viral genes and/or viral genomes to determine viral load) and/or culturing virus from samples. Such diagnostic tests are sold commercially, for example COBAS® AmpliPrep/COBAS® TaqMan® CMV Test and the COBAS® AMPLICOR CMV MONITOR Test (Roche) which can be used to diagnose HCMV infection and monitor antiviral therapy by the quantification of HCMV DNA. In certain embodiments the HCMV viral titer in an individual is reduced by about any of 100%, 95%, 90%, 85%, 80%, 75%, 70%, 65%, 60%, 55%, 50%, 45%, 40%, 35%, 30%, 25%, 20%, 15%, 10% or less relative to an untreated individual or relative to the viral titer of the same individual prior to treatment.

In additional embodiments, the organ or tissue transplanted may be any organ or tissue that is able to be transplanted from one individual to a second individual. For example, the organ transplanted may be, but is not limited to, a heart, kidney, liver, lung, pancreas, intestine, or thymus. Additionally, for example, the tissue transplanted may be, but is not limited to, hand, corneal, skin, face, islets of langerhans, bone marrow, stem cells, whole blood, platelets, serum, blood cells, blood vessels, heart valve, bone, bone progenitor cells, cartilage, ligaments, tendons, muscle lining.

In a further aspect, the invention provides compositions and pharmaceutical formulations comprising any of the anti-Complex I antibodies and/or gH antibodies provided herein, e.g., for use in any of the above therapeutic methods. In one embodiment, a pharmaceutical formulation comprises any of the anti-Complex I antibodies and/or anti-gH antibodies provided herein and a pharmaceutically acceptable carrier. In another embodiment, a pharmaceutical formulation comprises any of the anti-Complex I antibodies and/or anti-gH antibodies provided herein and at least one additional therapeutic agent, e.g., as described below.

The antibodies in the compositions of the invention can be used either alone or in combination with other agents in a therapy. For instance, the antibodies of the invention may be co-administered with at least one additional therapeutic agent. In certain embodiments, an additional therapeutic agent is a ganciclovir, valganciclovir, foscarnet, and/or cidofovir. In other embodiments an additional therapeutic agent is an additionally therapeutic isolated antibody.

Such combination therapies noted above encompass combined administration (where two or more therapeutic agents are included in the same or separate formulations), and separate administration, in which case, administration of the antibody compositions of the invention can occur prior to, simultaneously, and/or following, administration of the additional therapeutic agent and/or adjuvant.

Compositions of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Compositions of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The composition need not be, but is optionally formulated with one or more agents currently used to prevent or treat the disorder in question. The effective amount of such other agents depends on the amount of antibodies present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of the antibodies contain in the compositions of the invention (when used alone or in combination with one or more other additional therapeutic agents) will depend on the type of disease to be treated, the type of antibodies, the severity and course of the disease, whether the antibodies are administered for preventive or therapeutic purposes, previous therapy, the patient's clinical history and response to the antibodies, and the discretion of the attending physician. Each antibody included in the compositions described herein, is suitably administered to the patient at one time or over a series of treatments. Depending on the type and severity of the disease, about 1 μg/kg to 15 mg/kg (e.g. 0.1 mg/kg-10 mg/kg) of each antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of each antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. The progress of this therapy is easily monitored by conventional techniques and assays.

It is understood that any of the above formulations or therapeutic methods may be carried out using an immunoconjugate of the antibodies described herein in place of or in addition to an anti-Complex I antibody and/or an anti-gH antibody.

I. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

It is understood that any of the above articles of manufacture may include an immunoconjugate of the antibodies described herein in place of or in addition to an anti-Complex I antibody and/or an anti-gH antibody.

### III. Examples

The following are examples of methods and compositions of the invention. It is understood that various other embodiments may be practiced, given the general description provided above.

### Materials and Methods

Virus growth. VR1814 (Ravello Lab, Fondazione IRCCS Policlinico San Matteo, Pavia Italy) was expanded in either human fetal lung fibroblasts (MRC5) (American Type Culture Collection, ATCC; Manassas, Va.) cultured at passage 7-14 as instructed except in DMEM, or in PO human umbilical vein endothelial cells (HUVEC) (Lonza; Basel, Switzerland) at passage 4-6 as instructed and supernatant was concentrated, resuspended in complete media, and frozen. Complete media was composed of DMEM supplemented with 10% fetal calf serum, penicillin/streptomycin, L glutamine (all from Invitrogen; Carlsbad, Calif.) and 10 mM HEPES (Cellgro; Manassas, Va.). Assays were performed in 96 well plates in MRC5 and HUVEC cells and also in human retinal pigment epithelial cells (ARPE-19) (ATCC-cultured as instructed), monocyte derived macrophages (MDM), and cytotrophoblasts, which are placental epithelial cells. MDM were isolated from whole blood using RosetteSep Human Monocyte Enrichment Cocktail (Stemcell Technologies, Vancouver, BC, Canada) as instructed. Monocytes were then stimulated with 0.1 μg/ml lipolysacchardies (LPS) (Invivogen) and incubated in DMEM overnight. Platelets and unbound cells were washed away with PBS prior to infection. Cytotrophoblasts were isolated from 19 week placentas (Pereira Lab, UCSF using protocol from Librach et al., 1991, JCV 113:437-449) and seeded in 96-well tissue culture plates. Cytotrophoblasts preparations were assayed for the cytotrophoblast marker cytokeratin 7 (CK7) (Dako) and found to be more than 90% positive at the start of the infection.

The following HCMV strains were obtained from Dr. Jay Nelson (University of Oregon Health and Science University (OHSU); Portland, Oreg.): Adinis, Brown, Cano, Davis, Dement, Grunden, Harris, Keone, Lysistrata, NewRock, Phoebe, Powers, Salvo, Schmoe, Simpson, and Watkins. The following HCMV strains were obtained from Dr. Sunwen Chou (OHSU): C079, C323, C327, C336, C352, C353, and C359. Thawed virus was added to MRC5 fibroblasts and allowed to grow until 100% CPE was visible (approximately 10-12 days post-infection). Three days later, cells were scraped and the supernatant harvested and concentrated using ultracentrifugation. Dilutions of each strain were used to infect fresh fibroblasts in 96-well plates. Virus was allowed to infect for 18 hours, after which the cells were fixed with 100% ethanol. Staining with Mab810, an anti-IE antibody (Millipore; Billerica, Mass.) and immunofluorescence analysis were performed. Titers were calculated and used to determine the amount of virus necessary for a multiplicity of infection (MOI) of 1 for neutralization assays.

Neutralization assays. Neutralization assays were performed essentially as in Abai et al., 2007, J. Immunol Methods, 322:82-93, except that the assay is performed in DMEM (Gibco) and detected by immunofluorescence as described above. Briefly, antibody was serially diluted and mixed with virus diluted in complete media such that the final virion concentration resulted in approximately 1 infectious virus per cell (MOI=1) when mixed with media or with a non-inhibitory antibody. Antibody and virus was mixed and incubated at 37 degrees for one hour prior to addition to a confluent monolayer of MRC5s, ARPE-19s, HUVECs or monocyte-derived macrophages (MDM). Virus was allowed to infect for 18 hours after which time the cells were fixed with 100% ethanol. Cells were blocked in PBS, 2% BSA and then stained with an anti-HCMV IE antibody, Mab810 (Millipore) or Rabbit anti HCMV IE (Johnson Lab, Oregon Health Sciences University). Cells were washed with PBS and incubated with the appropriate AlexaFluor 488 and Hoechst stain (Invitrogen). Data from duplicate wells containing a given antibody concentration were averaged and compared with infection in the absence of antibody, which was set to 100%. Cells were imaged and counted using the ImageXpress® Micro™ and MetaXpress® (Molecular Devices). Data were log transformed, normalized, graphed and EC50s and EC90s calculated using Prism (GraphPad Software, La Jolla, Calif.). EC90 values were calculated from best-fit curves using EC50-curve fitting algorithm. The assay range of detection is 100-6.5×10⁵ infectious viral particles per well. Assay verification was seen between experiments, especially with differing multiplicity of infection of virus.

Amplification and clustering of clinical strains. Clinical strains of HCMV (from Oregon Health Sciences University) were grown on fibroblasts, supernatants harvested, and concentrated using ultracentrifugation. DNA was isolated from infected cells using DNeasy blood & tissue kit (Qiagen). HCMV gene primers were designed using an alignment of HCMV strains that are in the public domain. Conserved regions were chosen that were less than 500 bases apart. Sequences from clinical strains were viewed using Sequencher® and translated using MacVector. Alignment was performed by ClustalW and in Jalview Alignment Editor and trees constructed using nearest neighbor % identity.

Protein expression by baculovirus. In order to detect expressed proteins, rabbit polyclonal antibodies were produced by standard procedures. Each rabbit was immunized with a peptide corresponding to a HCMV protein. Peptides were as follows:

| (SEQ ID NO: 195) | |
|---|---|
| gH_871 | HPHHEYLSDLYTPCSSSGRRDHSLERLTR, |
| (SEQ ID NO: 196) | |
| gH_977 | CPHVWMPPQTTPHDWKGSHTTSGLHRPH, |
| (SEQ ID NO: 197) | |
| gL_873 | CGLPPELKQTRVNLPAHSRYGPQAVDAR, |
| (SEQ ID NO: 198) | |
| UL128_988 | VGLDQYLESVKKHKRLDVCRAKMGYMLQ, |
| (SEQ ID NO: 199) | |
| UL128_989 | RQVVHNKLTSCNYNPLYLEADGRIR, |
| (SEQ ID NO: 200) | |
| UL130_892 | RDYSVSRQVRLTFTEANNQTYTFCTHPN, |
| (SEQ ID NO: 201) | |
| UL130_892 | SPWFTLTANQNPSPPWSKLTYPKPHDC |
| and | |
| (SEQ ID NO: 202) | |
| UL131_993 | TAEKNDYYRVPHYWDACSRALPDQTRYK. |

Sera was purified by capture on the peptide-bound column and subsequent elution. For secreted proteins, a baculovirus construct was made for the extracellular domains of each individual HCMV protein. Each of the HCMV extracellular domains were fused to a baculovirus signal sequence and a 6×-His tag on the C-terminus. Baculovirus expression vectors were used to infect SF9 or Tini insect cells. Protein was harvested and gel filtrated, and examined by acrylamide gel electrophoresis and coomassie staining. Fractions containing all five proteins (gH, gL, UL128, UL130, and UL131) were pooled. The gB trimer construct was created by fusing gB S64-K115 to Q499-E655. When expressed by infection of insect cells, this construct resulted in gB protein that is not membrane anchored, formed trimers as expected, bound neutralizing and non-neutralizing gB antibodies, and was likely in its pre-fusion form. When gH and gL were co-expressed, the resulting gH/gL protein bound HB1 and the following antibodies which recognize both non-conformational and conformational gH epitopes: MSL-109, rabbit anti-gH, rabbit anti-gL (from David Johnson, OHSU, Ryckman et al., J. Virol. 82:60-70 (2008)), rabbit anti-gH_{—}977 and rabbit anti-gL_{—}873. The co-infection of insect cells by gH, gL, UL128, UL130 and UL131 resulted in a small amount of heterogeneous protein that bound hu8G8 and non-conformational and conformational antibodies including rabbit anti-gH, anti-gL, anti-UL128, anti-130, anti-131 and the rabbit polyclonal antibodies described above.

Construction of pRK—CMV vectors. For surface expression of full-length viral glycoproteins, three separate human expression plasmids were constructed. Individual HCMV genes were amplified from start to stop. gH, gL, gB were amplified from genomic DNA, and UL128, UL130, UL131 from cDNA and cloned first using PCR Blunt II TOPO (Invitrogen). Afterward, the genes were cloned into a Genentech mammalian expression vector (pRK-tk-Neo) with a “self-cleaving 2A peptide” sequence (Szymczak et al., Nat. Biotechnol. 22:589-94 (2004)) separating each HCMV gene and the last 3′ gene from the gene encoding eGFP. Three plasmids were constructed, one with gB and eGFP, one with gH, gL, and eGFP, and one with UL128, UL130, UL131, and eGFP. Plasmids were transfected into human embryonic kidney (HEK)-293T cells (American Type Culture Collection, ATCC; Manassas, Va.) using Lipofectamine 2000 (Invitrogen; Carlsbad, Calif.).

Cytogam® (HIG) depletion. For assay of the components of HIG that neutralize viral entry into epithelial cells, HEK-293T cells were transfected with either gB/eGFP or gH/gL/eGFP and UL128/UL130/UL131/eGFP or mock plasmid using Lipofectamine 2000 (Invitrogen) and incubated for 48 hours. Cells were dissociated using Accutase (Sigma), pelleted, and divided into 12 aliquots. Cytogam® was diluted to 20 μg/ml in PBS and 0.5% bovine serum albumin (BSA) and incubated in suspension with 3×10⁷ transfected cells for 1 hour. The Cytogam® was then serially transferred to fresh aliquots of 3×10⁷ transfected cells. Passage onto new cells was done until maximal specific deletion of antibodies was seen (six passages). ELISA assays were performed to detect depletion of certain HCMV specific antibodies. Maxisorp (NUNC) plates were coated with purified baculovirus produced gB, gH/gL, or gH/gL/UL128/UL130/UL131 in PBS and/or lysates of transfected HEK293T cells. Detection was determined with a goat anti-human IgG, Fcγ conjugated to horseradish peroxidase (Jackson Laboratory, Bar Harbor, Me.). The lower limit of detection is 0.08 μg/ml. The resulting HIG was then concentrated using 100 kD molecular weight cutoff concentrators (Centricon) for use in a neutralization assay as described above.

Affinity depletion columns were generated in the following manner. Proteins (1-2 mg of soluble gB or gH/gL) were extensively dialyzed against PBS and added to approximately 1 ml of Sterogene ALD Superflow resin equilibrated in PBS. Sodium cyanoborohydride (0.2 ml of 1 M solution) was added to chemically couple the proteins to the aldehyde containing resin and the reaction was allowed to proceed for overnight at 4° C. The individual resins were loaded into small columns and extensively washed with PBS to remove any unbound protein. Two milligrams of Cytogam® in a volume of 800 μl was loaded onto the columns and washed with PBS at a flow rate of 0.4 mL/min. Non-bound protein was collected in several 0.5 ml aliquots that were separately concentrated to approximately 100 μl in a spin concentrator with a molecular weight cutoff of 5000 daltons. Samples were sterile filtered and stored at 4° C. until assay.

FACS. pRK-CMV vectors, described previously, were transfected singly or at a 50:50 ratio for gH/gL and UL128-131 together using Lipofectamine 2000 (Invitrogen) into HEK293T cells. After 48 hours, cells were dissociated using Accutase (Sigma). All incubations of primary or secondary antibodies (Jackson Labs) and washes were in PBS, 2% FCS, 0.2% Sodium Azide (FACS buffer). After staining, cells were fixed in 2% paraformaldehyde in FACS buffer. Fluorescence analysis was done using FACS Calibur4 (Beckton Dickinson) and data processed using FlowJo Software (Tree Star Inc.).

Generation of Resistant Viral Mutants. To generate viral mutants resistant to the antibodies described herein, HCMV strain VR1814 was grown on epithelial cells in the presence of sub optimal concentrations of either the antibody MSL-109 (Aulitzky et al., J. Infect. Dis. 163:1344-47 (1991)), which was synthesized at Genentech, HB1, hu8G8, or a combination of HB1 and hu8G8 in ARPE 19 cells (American Type Culture Collection, ATCC; Manassas, Va.). The experiment was started in 24 well plates with three wells each of antibody at EC50, 2×EC50, or EC90 and a multiplicity of infection (MOI) of 0.5. Each week, half of each well volume was passaged onto new cells and concentration of antibody increased 1.5 fold or held steady. Typically, mutants emerged as single viral plaques by approximately passage 9. These viruses were grown in increasing concentration of antibody to a final concentration of 10×EC90. Subsequently, mutants were stocked and analyzed for resistance to HB1 and hu8G8 by neutralization assay as described above. The entire process was initiated four separate times (three wells per antibody concentration) on ARPE-19 cells and two separate times on MRC5 cells with only HB1 and MSL 109 (hu8G8 does not neutralize virus on MRC5 cells).

In order to generate additional resistance mutations, extracellular virus was treated with N-ethyl-N nitrosourea (ENU, Sigma-Aldrich; St. Louis, Mo.) or ultraviolet light (254λ Stratalinker, Stratagene; Santa Clara Calif.) and allowed to infect ARPE-19 or MRC5 cells in either a 24-well format (24 wells per treatment) or a 96-well format (72 wells per treatment). After infection (at MOI of 1 or 2), media was replaced with complete media with HB1 at EC100 or hu8G8 at EC100 or a combination of the two antibodies each at EC50, or ganciclovir (GCV, Sigma-Aldrich). Each week, supernatant was passaged to fresh cells with increasing concentrations of antibody or GCV. Growing virus was transferred to larger wells and stocked after 2-3 months. The entire process was initiated two separate times.

Sequencing of Glycoproteins. DNA was isolated from control or mutant virus infected cells or supernatant (DNA Blood/Tissue Extraction Kit, Qiagen; Valencia, Calif.). Primers were designed to conserved sequences across each gene according to an alignment of HCMV strains AD169, FIX, TB40E, Toledo, and Towne sequences available in the National Center for Biotechnology Information (NCBI) database. Glycoprotein H was amplified out of each clinical strain from the start codon through base 2196, just short of the stop codon. Glycoprotein B was amplified from the start to base 2686, just short of the stop codon. UL128, UL130, and UL131 were each amplified from start to stop according to the cDNA sequence obtained from Akter et al., J. Gen. Virol. 84:1117-22 (2003). The polymerase chain reaction (PCR) product was sequenced using dye terminator reactions and sequences aligned and trimmed (Sequencer).

Recapitulation of Mutations. pRK-CMV expression plasmids that contained the gH/gL genes or UL128/UL130/UL131 genes, as described above, were modified to substitute a single a mutation found in each resistance mutant. Each gH/gL plasmid was transfected (Lipofectamine 2000, Invitrogen; Carlsbad, Calif.) into HEK 293T cells (ATCC), allowed to express for 2 days, and then assessed by fluorescence activated cell sorting (FACS) analysis, as described above, for the ability of the surface expressed HCMV proteins to bind a control anti-gH antibody 10F8 or HB1. Each UL128/UL130/UL131 plasmid was co transfected with gH/gL plasmid, allowed to express for 2 days, then assessed for the ability of the surface expressed proteins to bind HB1, hu8G8, and control rabbit anti-UL131_{—}993 antibody. Analysis and images were generated using FlowJo (Treestar; Ashland, Oreg.).

Analysis of Viral Entry. Stocks of resistant and control strains (passaged in parallel on ARPE-19 cells in the absence of antibody) were grown and the supernatant was harvested (neat). Quantitative PCR (qPCR) was performed for pp65 DNA (pp65F TCGCGCCCGAAGAGG (SEQ ID NO:189), pp65R CGGCCGGATTGTGGATT (SEQ ID NO:190), Taqman probe CACCGACGAGGATTCCGACAACG (SEQ ID NO:191). To ascertain copy number, a standard curve was obtained with pp65 cloned into Zero Blunt PCR Cloning (Invitrogen). Dilutions of virus based on copy number were allowed to infect ARPE-19 and MRC5 cells for 18 hours prior to fixation and visualization. The infectious particles per DNA copy were calculated, normalized to the strain passed without antibody, and graphed using Excel version 14.1.2 (Microsoft; Redmond, Wash.).

### Example 1

### Production of Anti-Complex I and Anti-gH Antibodies

Production of Anti-Complex I Murine mAb 8G8. 2 groups of Balb/c mice (10 in each group) were immunized with whole UV inactivated (3000 mJ) HCMV (Strain VR1814) at a concentration of 1×10⁶ pfu per mouse twice a week for a total of 7 injections SC/IP. In the first group of animals, each mouse was primed with RIBI adjuvant and subsequently injected with HCMV in PBS. In the second group of mice, the animals were unprimed and then injected with HCMV in RIBI adjuvant. Test bleeds of the immunized mice were subjected to serum sample titration by ELISA and a virus neutralization assay as described above. The top 5 responding mice were chosen for production of hybridomas. Two separate sets of fusions were done using lymphocytes from the popliteal and inguinal nodes and mouse myeloma line X63-Ag8.653. Fused cells were plated in 96-well tissue culture plates (58 plates) and hybridoma selection using HAT media supplement (Sigma, St. Louis, Mo.) began one day post fusion. A total of 738 IgG+ hybridomas were screened using the virus neutralization assay on epithelial cells as described above. The EC50 (μg/ml) for HCMV Strain VR1814 for the resulting antibodies was tested for various cell types and compared to MSL-109 (an anti-gH antibody) and is shown in Table 2. The monoclonal antibody 8G8 was the most potent neutralizing antibody identified in the screen and was chosen for humanization and further characterization.

TABLE 2
EC50 (μg/ml): HCMV Strain VR1814

| | | Cell Type | | |
|---|---|---|---|---|
| | Target | Epithelial | Endothelial | Fibroblast |
| MSL-109 | gH | 0.1642 | 0.0764 | 0.3890 |
| Murine | Complex I | 0.0014 | 0.0007 | N/A |
| 8G8 | | | | |
| Murine | gH | 1.15 | 1.42 | 17.76 |
| 5H3 | | | | |
| Murine | gH | 0.1 | 0.1 | 1.11 |
| 10F8 | | | | |
| Murine | gH | 0.12 | 0.11 | 1 |
| 15H2 | | | | |
| Murine | Complex I | 0.03 | 0.07 | N/A |
| 354.1 | | | | |

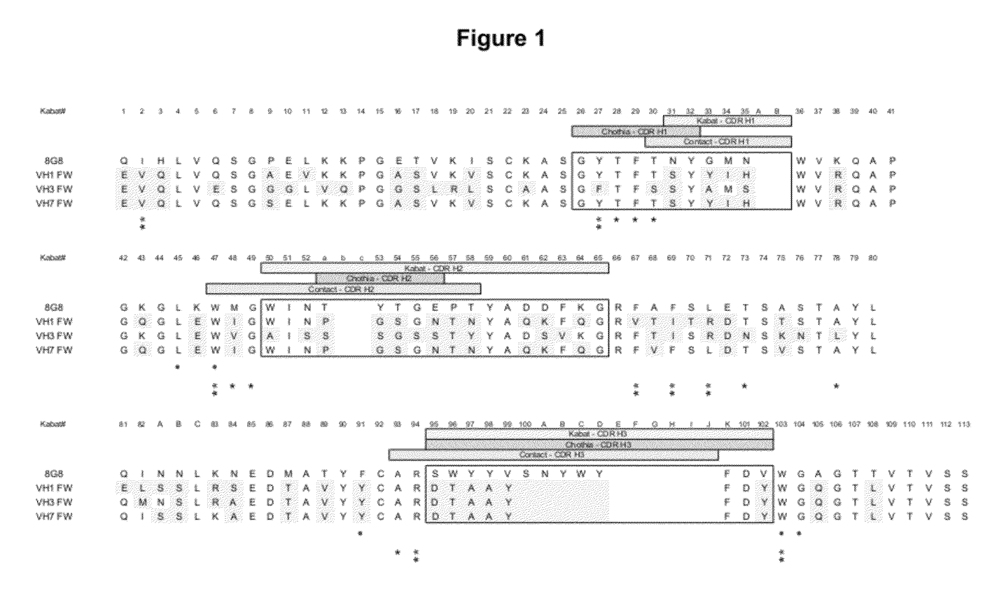

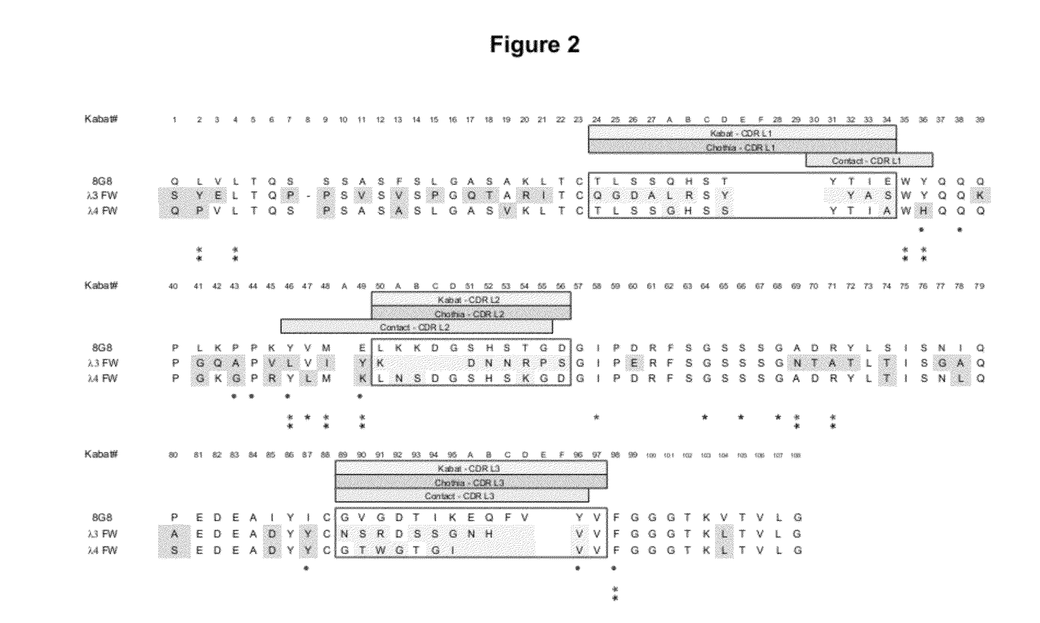

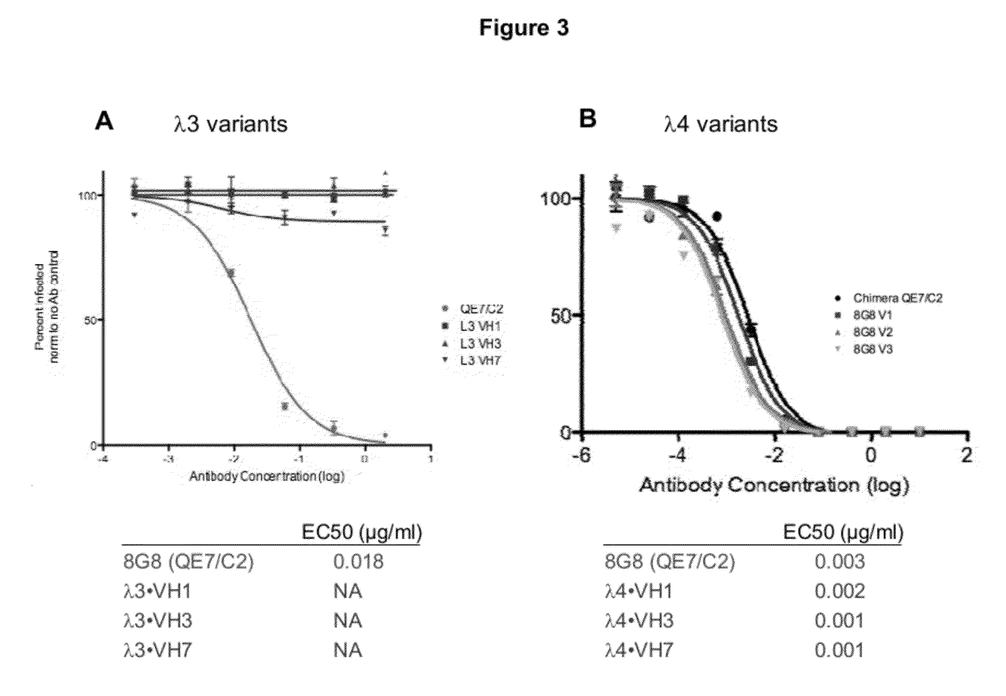

Humanization of Murine 8G8 mAb and Analysis. The murine hybridoma 8G8 was humanized by standard CDR graft using a lambda 3 or 4 light chain (FIG. 2) and either a VH1, VH3 or VH7 heavy chain framework (FIG. 1). For comparison, an alignment of consensus human λ germline sequences for λ3 and λ4 is shown in FIG. 2. Neutralization assays were performed comparing an 8G8 human/murine chimeric antibody (QE7/C2) with the 8G8 VH1, VH3 or VH7 humanized heavy chains combined with either the 8G8 λ3 or λ4 light chains. It was found that the λ4 variants, but not the λ3 variants neutralized HCMV (FIG. 3).

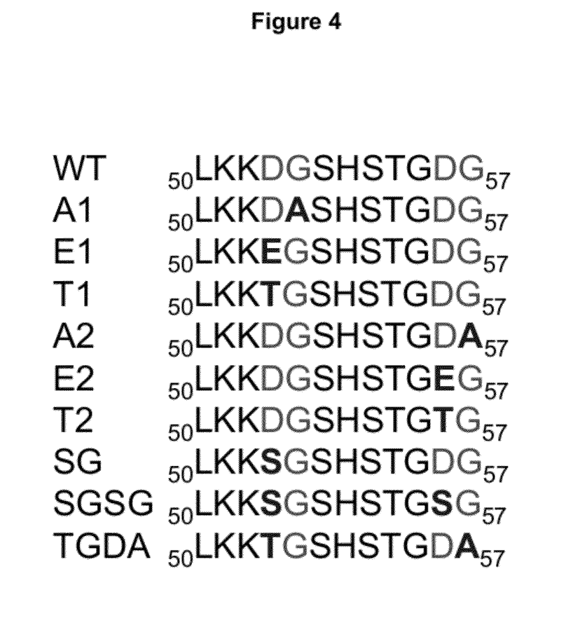

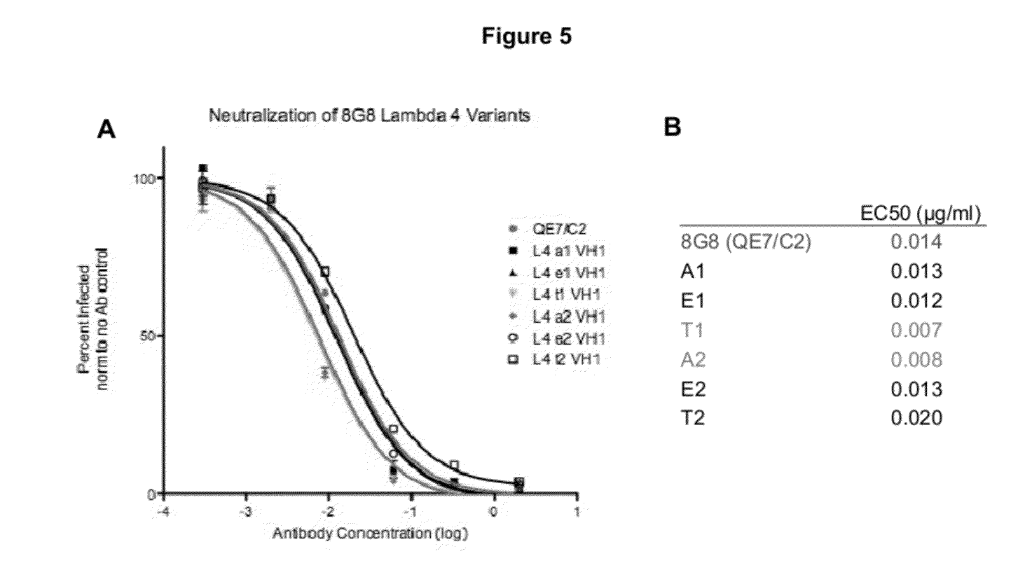

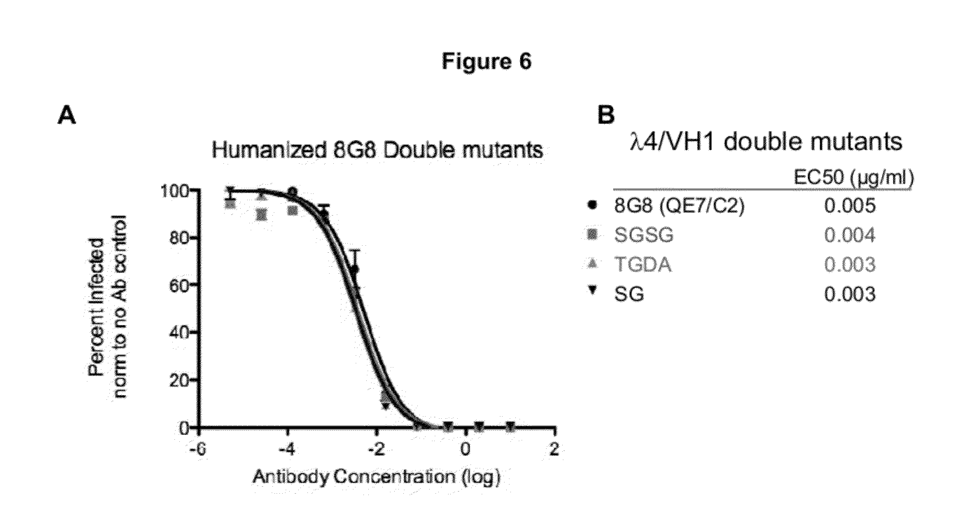

The HVR-L2 of λ4 was mutated as shown in FIG. 4 to introduce substitutions at amino acids 50C, 50D, 56, as well as an amino acid substitution at amino acid 57 (the first amino acid of FR3), according to Kabat numbering, to provide stability for the antibody. The various mutated light chains were then combined with the 8G8 human VH1 chain and the resulting antibodies were tested in neutralization assays as described above. Antibodies with single amino acid substitutions all showed good neutralization activity (i.e. A1, E1, T1, A2, E2, and T2) (FIG. 5). Likewise, all antibodies containing two amino acid substitutions showed good neutralization activity (i.e., SGSG and TGDA). The single mutant SG was included as a comparison control, and it also showed good neutralization activity. (FIG. 6).

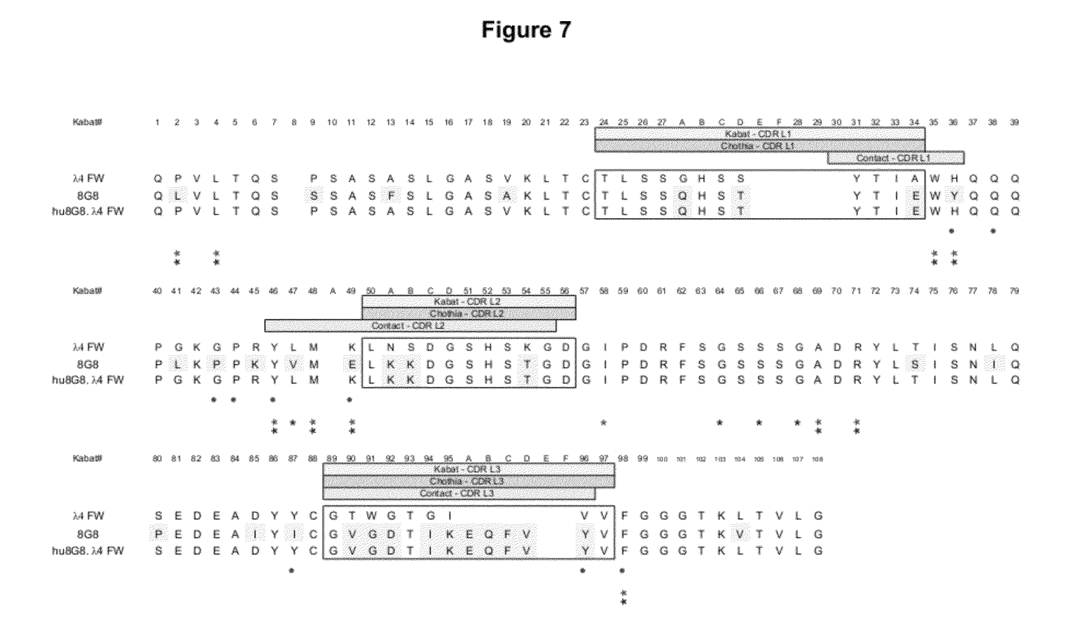

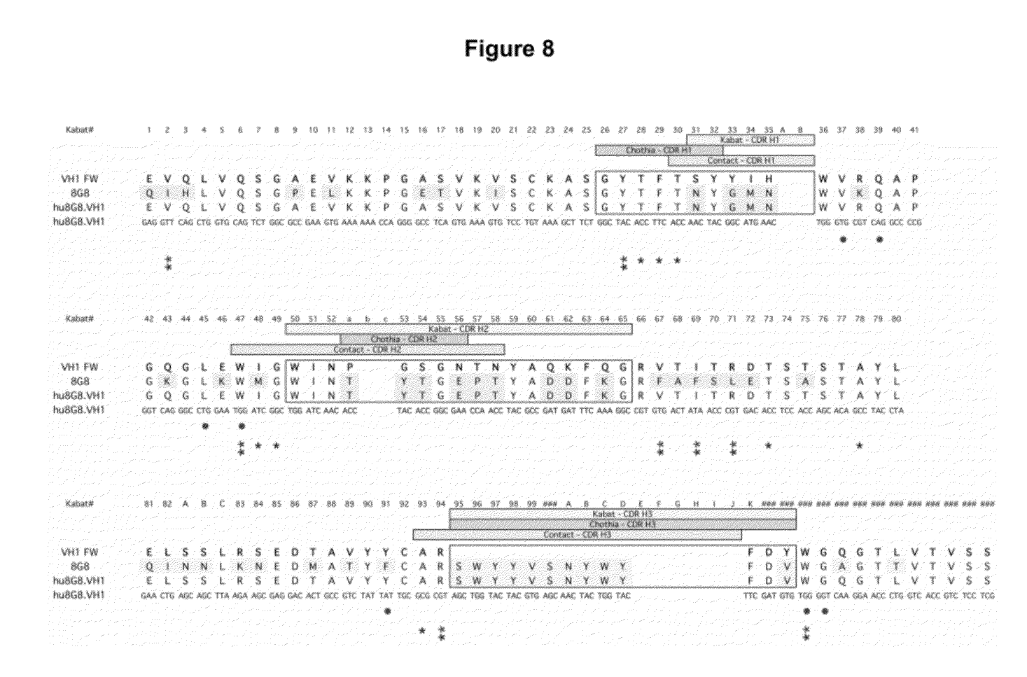

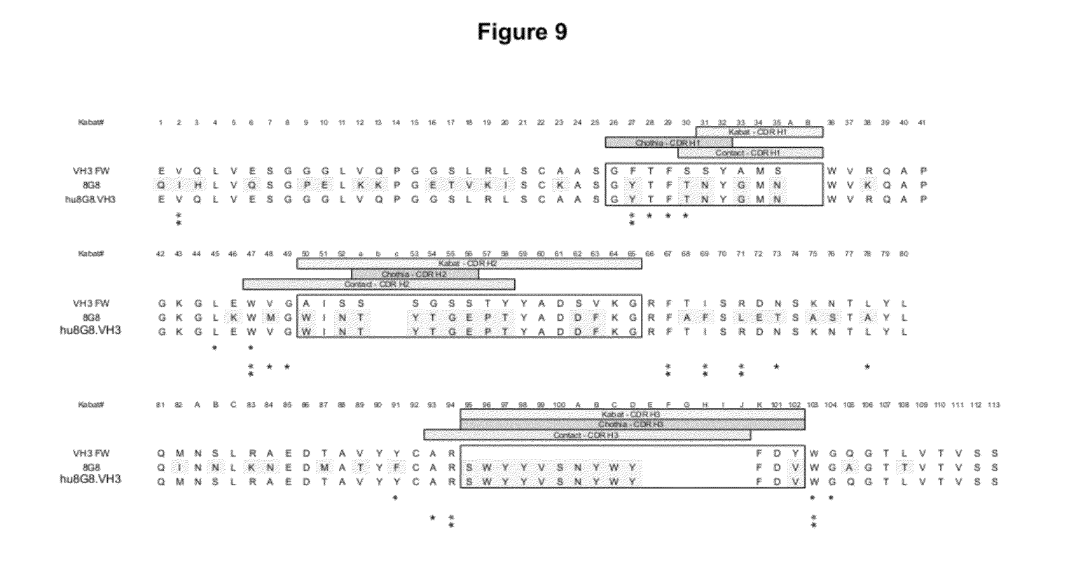

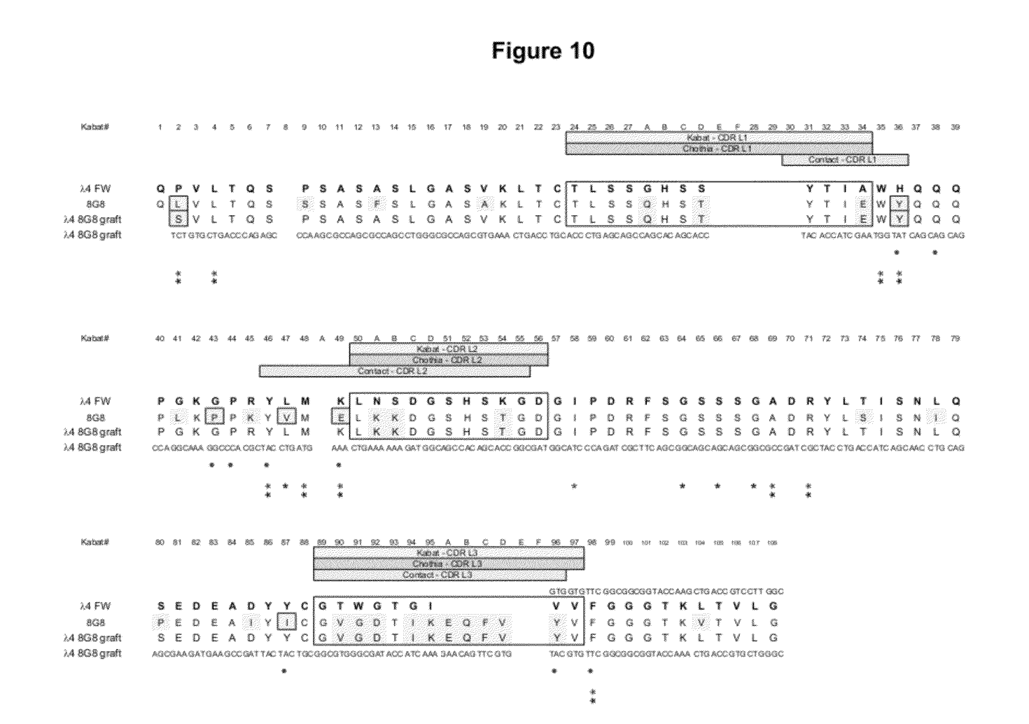

A humanized 8G8 λ4 antibody sequence is shown in FIG. 7 (hu8G8.λ4 FW). FIG. 8 shows the sequence of a humanized 8G8 VH1 sequence (hu8G8.VH1) while FIG. 9 shows the sequence of a humanized 8G8 VH3 sequence (hu8G8.VH3). FIG. 10 shows a humanized 8G8 λ4 antibody sequence in which the first two amino acids (QP) have been modified such that the polypeptide begins with serine (Q is deleted and L is mutated to S) and amino acid 36 retains the murine amino acid (Y). The polypeptide sequence of this antibody is shown as λ4 8G8 graft. A representative nucleic acid sequence encoding the polypeptide is shown below the polypeptide sequence.

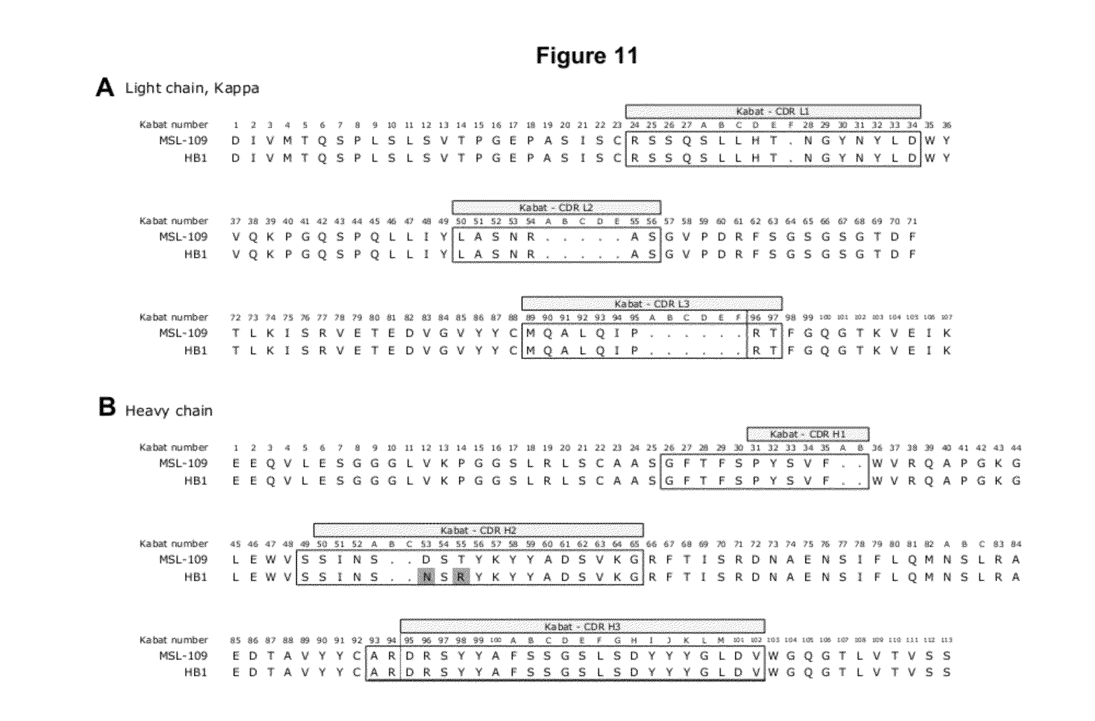

Affinity Maturation of Anti-gH Antibody. The monoclonal antibody MSL-109 was synthesized using the antibody sequence for the variable heavy and variable light chain sequences of MSL-109 published in PCT Publication No. WO 94/16730, published Aug. 4, 1994, and incorporated herein by reference in its entirety. The amino acid sequences of the MSL-109 VH and VL chains are shown in FIG. 11 (VL, SEQ ID NO:90; VH, SEQ ID NO:92). The MSL-109 antibody was based on an IgG1 framework containing heavy chain VH3 and light chain Vκ2. The recombinant DNA encoding this antibody was cloned into CHO cells.

Antibody MSL-109 was affinity matured by randomization of complementary determining regions (CDRs) followed by selection of binders by phage display with progressively limiting concentrations of biotinylated gH/gL. Each position of the CDRs was randomized by oligonucleotide-directed mutagenesis with an “NNK” codon, where N is any of the four natural nucleotides, and K is 50% thymine and 50% guanine. The NNK codon can encode any of the 20 natural amino acids. Libraries for the light chain and heavy chain were made separately, and each of the 3 CDRs of each chain was randomized at the same time. This results in clones that have 0 to 3 random amino acid changes in each chain, with up to one mutation in each CDR. Libraries were made in a phage Fab fragment display vector and by standard methods. Binding clones were selected by incubating the phage display libraries with 1 and 0.1 nM biotinylated gH/gL in successive rounds of selection, and then competed with either 100 nM gH/gL or MSL-109 IgG to reduce binding of the lower affinity clones to gH/gL. Bound clones were captured on ELISA plates coated with neutravidin or streptavidin, washed and eluted in 10 mM HCl for 10 minutes at room temperature. The eluted phage was neutralized with 1/10 volume of 1 M Tris pH 8.0 and used to infect E. coli for amplification for the next round of selection. Clones from the second round of selection were sequenced to determine mutations that are frequent in selected phage. Clones with favored mutations were tested by a competition phage ELISA.

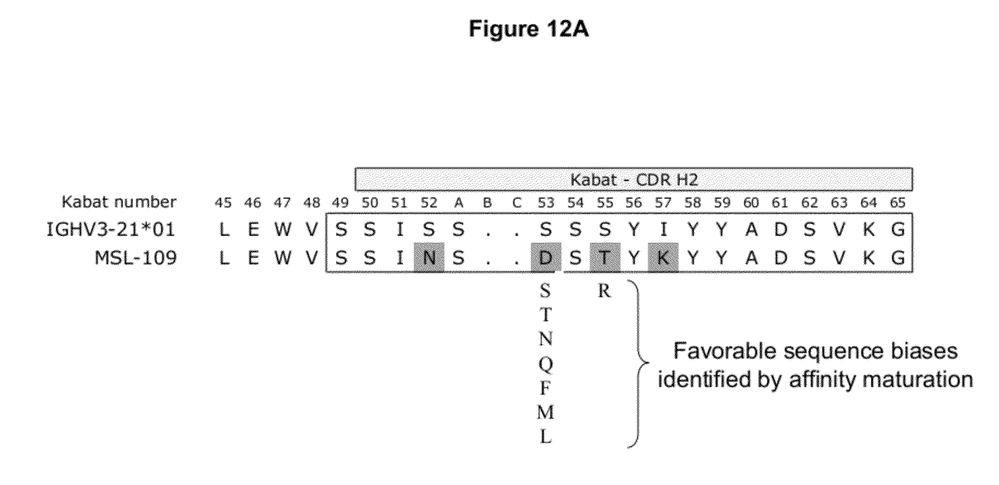

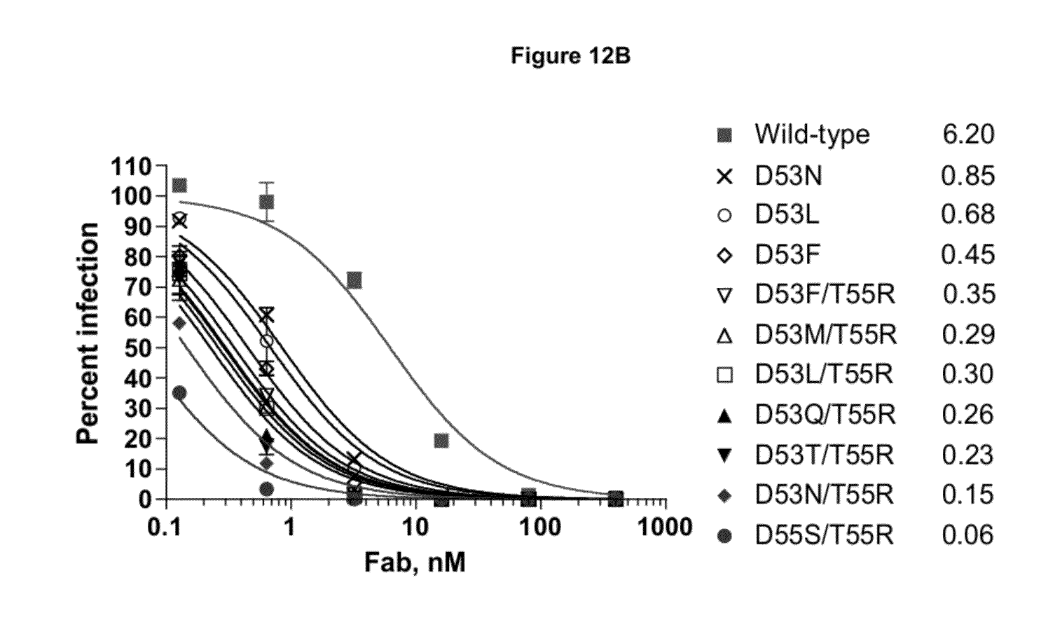

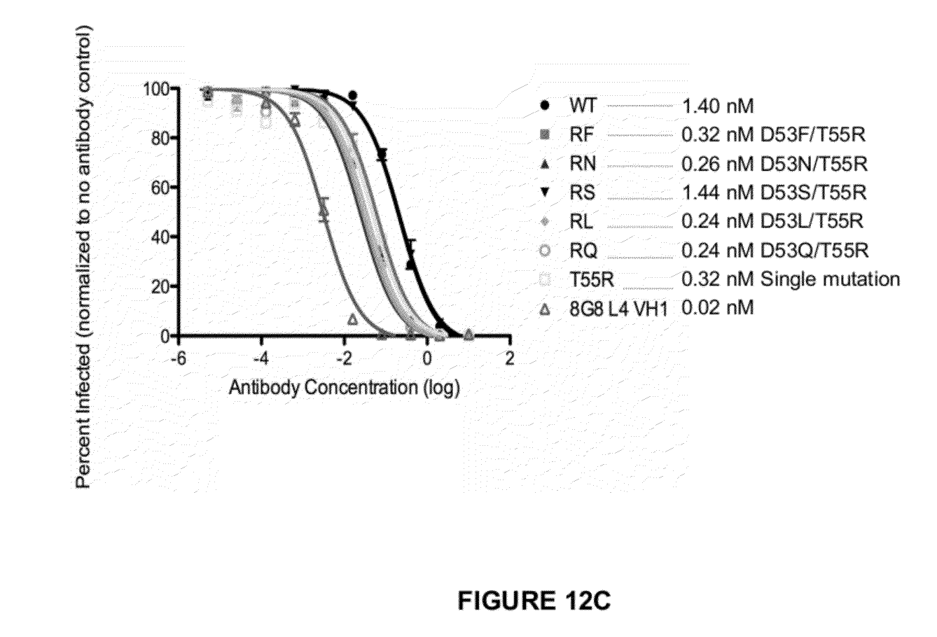

IgG and Fab fragments of mutant MSL-109 with individual or combined mutations in heavy chain Kabat positions 53 and 55 were expressed and tested for in vitro neutralization of CMV. Amino acid substitutions at amino acid 53 (replacing D53 with S, I, N, Q, F, M, L, G, H, K, W, Y, V or A) alone or in combination with an amino acid substitution at amino acid 55 (replacing T55 with either R or K) provided antibodies with improved neutralization capability (FIGS. 12B and 12C). A schematic of some of these changes is shown in FIG. 12A. Additionally, the amino acid N52 in MSL-109 may be replaced with S. This substitution does not affect potency but allows S in position 53 without glycosylation of position 52. There are 89 possible combinations for heavy chain variable sequences using the various amino acid substitutions at amino acid 53 and/or amino acid position 55 (SEQ ID NOs:87, 88, 89, and 96-182). A consensus sequence is provided as SEQ ID NO:94). The Fab fragments of these anti-gH antibodies were measured in phage display ELISA assays for affinity to the gH/gL dimer produced in baculovirus. Specifically phage clones displaying MSL-109 variant Fab fragments were incubated with serially diluted gH/gL and incubated for 1 hour at room temperature. The unbound phage was detected by incubating the mixture to ELISA plate wells coated with gH/gL for 10 minutes at room temperature. Plates were washed with PBS-T, and phage bound to the immobilized gH/gL were detected by incubation with an anti-M13 HRP conjugate for 30 minutes followed by wash and developing with TMB substrate. The IC50, point where 50% of the phage in the phage-gH/gL mixture was free, was calculated by non-linear regression. The IC50s were in the range of 0.01-0.1 nM. Individual affinities for selected variants are shown in Table 3.

TABLE 3

| | | Phage ELISA |
|---|---|---|
| | Variants | IC₅₀ (nM) |
| H2 Single | WT (MSL-109) | 0.53 |
| Mutants | H2-D53L | 0.02 |
| | H2-D53M | 0.05 |
| | H2-D53N | 0.06 |
| | H2-D53S | 0.1 |
| | H2-D53T | 0.06 |
| H2 Double | H2-T55R | 0.17 |
| Mutants | H2-D53F/T55R | 0.01 |
| | H2-D53L/T55R | 0.02 |
| | H2-D53M/T55R | 0.07 |
| | H2-D53N/T55R (HB1) | 0.05 |
| | H2-D53Q/T55R (HB2) | 0.01 |
| | H2-D53S/T55R | 0.03 |
| | H2-D53T/T55R | 0.03 |

Surprisingly, amino acid changes at the VH2 HVR produced antibodies with dramatically higher binding and neutralization ability. For example HB1 (D53N/T55R) had a 10-fold increased affinity for gH/gL than MSL-109 as shown by phage ELISA (Table 3). When expressed as Fab fragments in E. coli, HB1 (D53N/T55R) is approximately 40-fold more potent for inhibition of HCMV entry into ARPE-19 cells (i.e., EC50=0.15 nM vs. 6.2 nM) than the parental MSL-109 antibody as shown by neutralization assays (FIG. 12B). Moreover, HB1 (D53N/T55R), when expressed as full-length IgG in CHO cells, is approximately 6-fold more potent for inhibition of HCMV entry into ARPE-19 cells as shown by neutralization assay (Table 4, FIG. 12C). The EC50s and EC90s (μg/ml) for the HB1 antibody compared to the MSL-109 antibody in neutralization assays (one representative experiment), on various cells types is shown in the Table 4 below.

TABLE 4

| | Epithelial | Epithelial | Endothelial | Endothelial | MDM | MDM | Fibroblast | Fibroblast |
|---|---|---|---|---|---|---|---|---|
| | EC50 | EC90 | EC50 | EC90 | EC50 | EC90 | EC50 | EC90 |
| MSL-109 | 0.3 | 2 | 0.48 | 2.9 | 0.04 | 0.42 | 0.73 | 4.8 |
| HB1 | 0.05 | 0.41 | 0.03 | 0.28 | 0.03 | 0.19 | 0.11 | 0.77 |

### Example 2

### Antibody Functional Studies

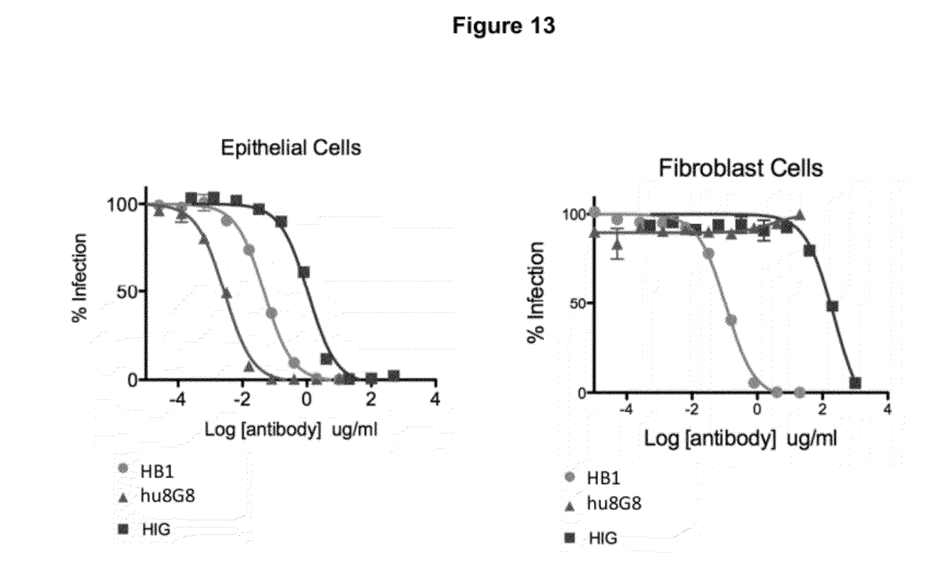

HB1 (D53N/T55R) and hu8G8 were compared to HIG in neutralization assays for the ability to block HCMV viral entry into epithelial cells, endothelial cells, macrophages and fibroblasts. hu8G8 has an EC50 of 0.003 μg/ml (0.02 nM) on epithelial cells, 0.004 μg/ml (0.03 nM) on endothelial cells, and 0.001 μg/ml (0.006 nM) on monocytes. hu8G8 is at least 8× more potent than HB1 at neutralizing HCMV on each of these cell types. However, as expected, hu8G8 does not block viral entry into fibroblasts, whereas HB1 does so with an EC50 of 0.11 μg/ml (0.7 nm) (see FIG. 13).

HIG has been reported to prevent HCMV fetal infection and/or disease when given to pregnant women with primary HCMV infection (Nigro et al., 2005), suggesting the ability of CMV-specific antibodies to confer protection to the developing fetus. When evaluated by neutralization assay, HIG was found to neutralize viral entry into all tested cells types, but with a potency far less than either of the monoclonal antibodies (see FIG. 13). This comparatively low potency is due to the polyclonal nature of HIG which only has a small fraction of proteins with anti-CMV neutralization activity.

### Example 3

### HIG Depletion Studies

In order to identify the neutralizing antibody component in hyperimmune globulin, HIG was depleted of anti-gB antibodies or anti-Complex I (gH/gL/UL128/UL130/UL131) antibodies using HEK293T cells transfected with gB or Complex I by six serial incubations. The Cytogam®(HIG) depletions were performed according to the method described above. Analysis of the absorbed serum showed that >95% of the antibodies reactive with gB-transfected cells was absorbed by this procedure, compared to 0% on control cells, as assayed by purified gB ELISA. However, only about 45% of the antibodies reactive with cells transfected with Complex I had been absorbed, as compared to 0% on control cells, as assayed by ELISA with lysates from transfected HEK293T cells, as described above.

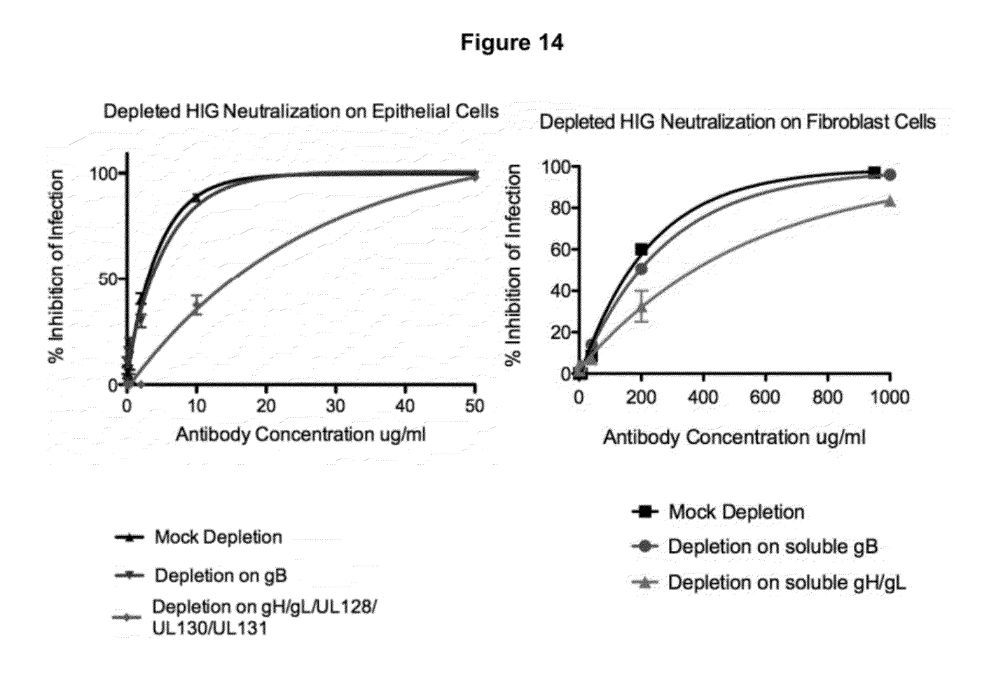

The depleted HIG was then used in neutralization assays to determine the effect of depletion on preventing viral entry into epithelial cells. Serial dilutions of the absorbed HIG preparations compared to mock-absorbed HIG was used in neutralization assays as described above. The results of these experiments are shown in FIG. 14. Antibodies against gB do not appear to significantly contribute to the neutralization ability of HIG on epithelial cells, whereas antibodies against Complex I appear to significantly contribute to the neutralizing activity of HIG. Removal of Complex I specific antibodies decreased the neutralization ability (EC50) of HIG by about 85% when tested on epithelial cells

Assay of the depleted HIG on fibroblasts was not possible because of the very high concentration needed to detect neutralization. The EC50 of HIG is approximately 500 μg/ml on this cell type. Since the UL128, UL130 and UL131 proteins are not required for entry into fibroblasts, baculovirus expressed gB or gH/gL, bound to a column, as described above, was used to specifically deplete the antibodies from HIG specific for these proteins/complexes. With nearly complete depletion of anti gB antibodies (95% depletion of gB antibodies on gB column versus 0% depletion of gB antibodies on gH/gL column), no neutralization shift was observed. However, with the majority of anti gH/gL antibodies depleted (84% depletion of gH/gL antibodies on gH/gL column versus 0% depletion of gH/gL antibodies on gB column), a 65% reduction in EC50 was seen (see FIG. 14).

From these data it was concluded that the major neutralizing antibodies in HIG are directed at the gH/gL/UL128/UL130/UL131 complex. Specifically, Complex I neutralizing antibodies are the major neutralizing antibodies for epithelial cell entry in HIG. Additionally, gH/gL antibodies in HIG have a dominant role in inhibition of viral entry into fibroblasts. These experiments show little role for anti-gB antibodies in HIG neutralization.

By absorbing HIG using baculovirus expressed gB, gH/gL and gH/gL/UL128/UL130/UL131, it was determined by ELISA, that approximately 1% of HIG was gB reactive, while approximately 0.1-0.2% was reactive with either Complex I or gH/gL. By knowing the concentration of complex-specific antibodies in HIG, the neutralization potency of those complex-specific antibodies could be calculated by correcting the neutralization potency of intact HIG for the percentage of IgG that was actually directed to the relevant complex leading to neutralization (e.g., 810 μg/ml×0.1-0.2=0.8-1.6 on fibroblasts), as shown in Table 5 below.

TABLE 5
EC₉₀ (μg/ml)

| Neutralizing | | Epithelial | Endothelial | Macro- | Fibro- |
|---|---|---|---|---|---|
| Agent | Target | Cells | Cells | phages | blasts |
| HCMV- | HCMV + | 0.01-0.02 | 0.01-0.02 | 0.004-0.01 | 0.8-1.6 |
| HIG† | unknown | | | | |
| HB1 | gH | 0.4 | 0.28 | 0.19 | 0.78 |
| HB2 | gH | 0.41 | 0.44 | 0.21 | 0.78 |
| HCMV-HIG | HCMV + | 8.0 | 11.6 | 3.8 | 810 |
| | unknown | | | | |
| Humanized | Complex I | 0.02 | 0.04 | 0.010 | n/a |
| 8G8 with | | | | | |
| VH1 | | | | | |
| Humanized | Complex I | 0.010 | 0.7 | 0.010 | n/a |
| 8G8 with | | | | | |
| VH3 | | | | | |

†EC90 values were adjusted for concentration of gH/gL/UL128/UL130/UL131 antibodies in HIG (0.1-0.2%).

As shown above in Table 5, a combination of HB1 and humanized 8G8 (either VH1 or VH3) is able to approximate the neutralization potency of HIG on all cell types tested in neutralization assays. Cells were infected at the following multiplicity of infection (MOI) of HCMV: epithelial cells MOI=1, endothelial cells MOI=1, macrophages MOI=0.5 and fibroblasts MOI=1. HB1 has comparable neutralizing potency to HIG (as corrected for the amount of HIG that is Complex I-specific) for inhibition of infection on fibroblasts, but does not provide adequate potency on epithelial cells, endothelial cells, or macrophages. Humanized 8G8 (VH1) and (VH3) has comparable neutralizing potency to HIG (as corrected for the amount of HIG that is Complex I-specific) on epithelial cells, endothelial cells and macrophages. However, it fails to neutralize infection of fibroblasts. Thus, the combination of antibodies provides neutralization of HCMV comparable to that of HIG, adjusted for Complex I-specific antibodies, on all cell types tested.

The ability of HB1 and hu8G8 with VH1 to neutralize HCMV all various cell types, was tested again in neutralization assays and compared to the calculated and actual neutralization potency of HIG. Cells were infected with the following MOI of HCMV: epithelial cells MOI=1, endothelial cells MOI=0.25, macrophages MOI=0.25 and fibroblasts MOI=0.8. The results of this experiment are shown below in Table 6. Average EC90's from this experiment and the results show in Table 5 are shown below in the shaded boxes in Table 6.

TABLE 6
EC₉₀ (μg/ml)

| Neutralizing | | Epithelial | Endothelial | Macro- | Fibro- |
|---|---|---|---|---|---|
| Agent | Target | Cells | Cells | phages | blasts |
| HCMV- | HCMV + | 0.01-0.02 | 0.007-0.014 | 0.01-0.02 | 0.4-0.7 |
| HIG† | unknown | 0.01-0.02 | 0.01-0.02 | 0.01-0.02 | 0.6-1.2 |
| HB1 | gH | 0.18 | 0.08 | 0.14 | 0.25 |
| | | 0.29 | 0.18 | 0.17 | 0.52 |
| HCMV-HIG | HCMV + | 10.2 | 6.7 | 10.9 | 370 |
| | unknown | 9.1 | 9.1 | 7.4 | 590 |
| Humanized | Complex I | 0.01 | 0.008 | 0.009 | n/a |
| 8G8 with | | 0.02 | 0.02 | 0.01 | |
| VH1 | | | | | |

†HIG adjusted for contribution of gH/gL/UL128/UL130/UL131 antibodies.

### Example 4

### Neutralization of HCMV Clinical Isolates

gH, gL, UL128, UL130, and UL131 genes were sequenced from over 20 clinical isolates obtained from two laboratories at Oregon Health Sciences University and compiled with additional, publically available sequences. The publically available sequences were generated from strains originating from the United States, Europe, and Japan.

Cells infected by each strain were lysed and DNA was extracted using the DNA blood/tissue extraction kit (Qiagen; Germantown, Md.). Primers were designed to conserved sequences according to an alignment of AD169, FIX, TB40E, Toledo, and Towne sequences available in the National Center for Biotechnology Information (NCBI) database. Glycoprotein gH was amplified out of each clinical strain from the start codon through base 2196, just short of the stop codon. The polymerase chain reaction (PCR) product was sequenced using dye terminator reactions at Genentech. Sequences were aligned and trimmed (Sequencer). Additional gH sequences were obtained from the NCBI database by using one accession number from Chou et al., J. Infect. Dis. 166:604-7 (1992) and then obtaining “Related Sequences.” In total, glycoprotein sequences from 57 strains were clustered (ClustalW, European Bioinformatics Institute (EBI); Cambridgeshire, England) after the signal peptide was removed, and aligned into a tree using average distance by percentage identity (JalView; Waterhouse et al 2009).

The sequencing results indicated a 1% variation in sequence in UL128, UL130, and UL131 across the clinical isolates (after removing the signal peptide). This finding was consistent with a study in Europe that demonstrated that UL128, UL130, and UL131 were highly conserved among pregnant women with a primary HCMV infection (Baldanti et al., Arch. Virol. 151:1225-33 (2006)).

gH is at least 95% identical among all strains at the protein level (after removing the signal peptide). A phylogenetic tree with two distinct branches was constructed (data not shown). The tree is in agreement with a previous report, in which gH protein sequences segregated into two phylogenetic groups (Chou, J. Infect. Dis. 166:604-7 (1992)). Also in accordance with the literature, HCMV isolates in both branches were not geographically distinct (i.e. strains isolated in Japan could be found in both branches) (Pignatelli, J. Gen. Virol. 84:647-655 (2003)).

The ability of HB1 (D53N/T55R) to neutralize infectivity of a diverse panel of clinical isolates of HCMV on fibroblasts was tested. Table 7 shows the effectiveness of HB1 compared to HIG. HB1 was found to neutralize each of the strains of HCMV representing the greatest gH sequence diversity as well or better than HIG (when corrected for the amount of HIG that is gH/gL/UL128/UL130/UL131-specific). The results of neutralization assays using the HCMV strains Dement, Adinis and VR1814 at various MOI, in multiple experiments are also shown in Table 7 below.

TABLE 7

| Strain Name | HB1 (D53N/T55R) | HIG | HIG |
|---|---|---|---|
| (branch #) | EC90 (μg/ml) | EC90 (μg/ml)* | EC90 (μg/ml) |
| Cano (1) | 0.27 | 0.3-0.7 | 355 |
| Keone (1) | 0.12 | 0.1-0.2 | 104 |
| New Rock (1) | 0.18 | 0.12-0.24 | 119 |
| TR (1) | 0.59 | 0.3-0.7 | 350 |
| Brown (2) | 0.76 | 0.4-0.9 | 442 |
| Dement (2) | 0.78 | 0.8-1.6 | 80 |
| | 0.08 | 0.15-0.3 | 154 |
| Adinis (2) | 2.0 | 0.74-1.5 | 233 |
| | 0.08 | 0.1-0.2 | 120 |
| Grunden (2) | 0.08 | 0.14-0.28 | 137 |
| Harris (2) | 0.32 | 0.3-0.6 | 305 |
| Phoebe (2) | 0.04 | 0.1-0.2 | 115 |
| VR1814 (2) | 0.08 | 0.1-0.3 | 790 |
| | 0.78 | 0.8-1.6 | |
| | 0.25 | 0.4-0.7 | 370 |

*EC90 values were adjusted for concentration of gH/gL/UL128/UL130/UL131 antibodies in HCMV-HIG (0.1-0.2%)

### Example 5

### Specificity of Antibodies

The antigen specificity of HB1 and hu8G8 was assessed. Plasmids containing viral glycoproteins were constructed such that each protein was expressed in equal stoichiometry by separating each gene with a “self cleaving 2A peptide” (Szymczak et al., Nat. Biotechnol. 22:589-94 (2004)). Plasmids contained full-length genes of either gB/eGFP, gH/gL/eGFP, or UL128/UL130/UL131/eGFP (cloned from cDNA). Plasmids were transfected into human embryonic kidney (HEK) 293T cells (American Type Culture Collection, ATCC; Manassas, Va.) using Lipofectamine 2000 (Invitrogen; Carlsbad, Calif.) to express CMV glycoproteins at their surface. After 2 days, cells were dissociated and stained with saturating primary antibody HB1, hu8G8, anti gB, affinity-purified rabbit anti UL131_{—}933 or affinity-purified rabbit anti gH_{—}977. Cells were stained with appropriate secondary antibody conjugated to allophycocyanin (APC, Jackson ImmunoResearch; West Grove, Pa.). Fluorescence of individual cells was measured using FACSCalibur (BD Biosciences; San Jose, Calif.) and analyzed using FlowJo software (Tree Star; Ashland, Oreg.). GFP positive cells (those expressing the CMV transgenes) were selectively graphed to show antibody binding.

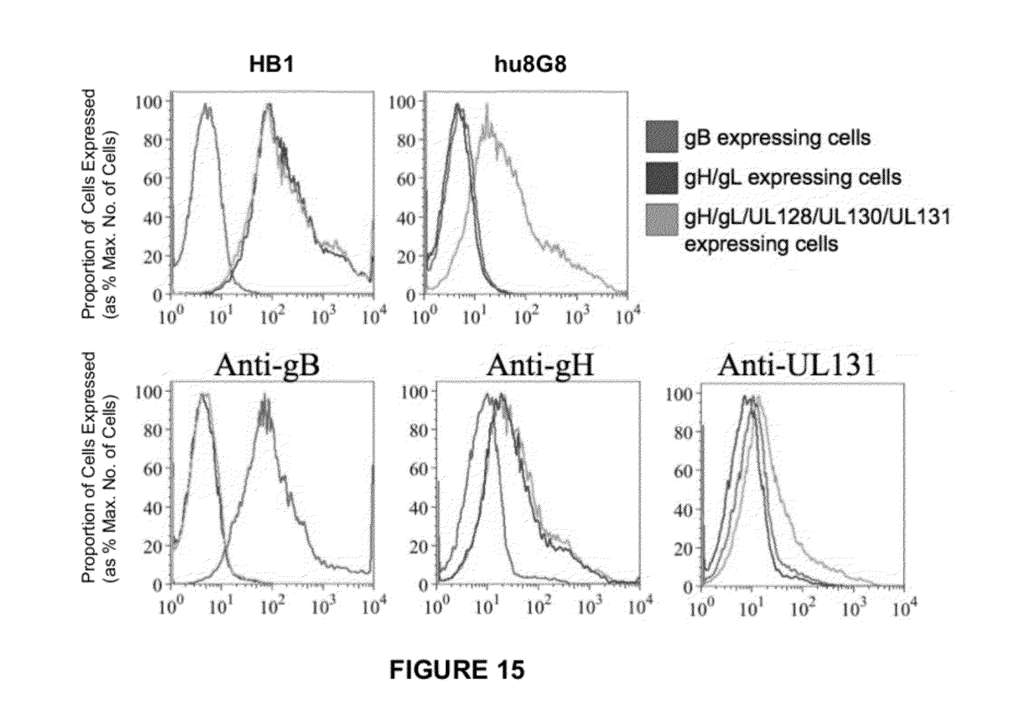

As shown in FIG. 15, HB1 reacted to cells expressing gH/gL alone or in complex with UL128/UL130/UL131. Hu8G8 reacted only to cells expressing gH/gL/UL128/UL130/UL131 (FIG. 15) and not to cells expressing gH/gL alone or gH/gL/gO (data not shown). Neither antibody reacted to cells expressing gB. Thus, HB1 recognizes an epitope on gH which is present in the gH/gL complex and in Complex I. The hu8G8 antibody binds to an epitope within the five envelope proteins which form Complex I, but does not bind to gH/gL/gO or gH/gL alone.

### Example 6

### Assessment of Synergy or Antagonism

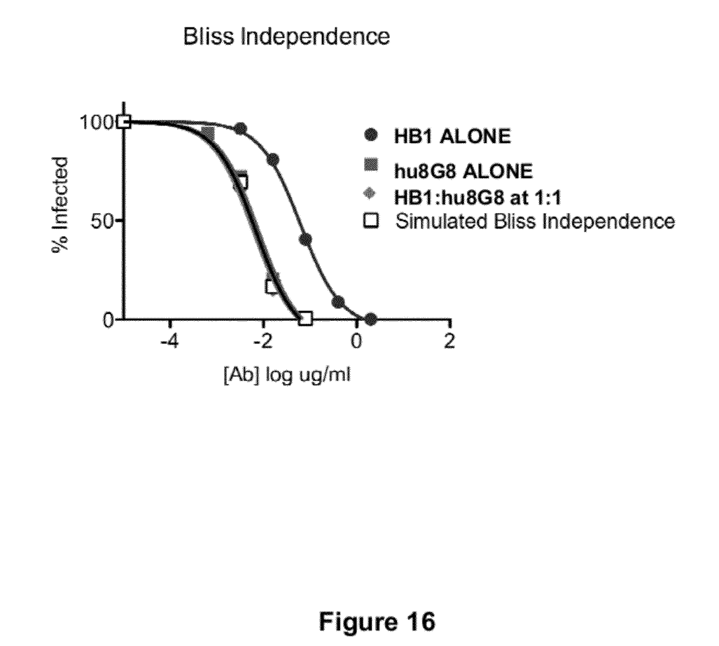

HB1 and hu8G8 were tested in combination in a viral neutralization assay to determine if a difference in potency existed when the two antibodies were combined. Since the antibodies have different targets, and presumably act independently in blocking viral entry, it was assumed that the effects of HB1 and hu8G8 are additive. Thus, the Bliss independence equation was applied (The Combined response C for two single compounds with effects A and B is C=A+B−A*B). To this end, HB1 and hu8G8 were mixed in a 1:1 ratio and tested in a dilution series in a viral neutralization assay on epithelial cells and EC50s were calculated as described above. HB1 did not enhance or diminish the potency of hu8G8 on epithelial cells and the 1:1 curve precisely overlapped the simulated Bliss independence curve, suggesting additivity rather than synergy (see FIG. 16 and Table 8). Likewise, hu8G8 did not alter the potency of HB1 on fibroblasts, as expected since hu8G8 does not block HCMV entry into fibroblasts (data not shown). Thus, HB1 and hu8G8 do not demonstrate any antagonism or synergy at a 1:1 ratio.

TABLE 8

| | EC50 on epithelial |
|---|---|
| mAB | cells (μl/ml) |
| HB1 alone | 0.063 |
| hu8G8 alone | 0.0080 |
| HB1:hu8G8 at 1:1 | 0.0060 |
| Simulated Bliss Independence | 0.0069 |

To assess whether HB1 and hu8G8 demonstrated synergy or antagonism at a wide range of ratios, a pair-wise “checker board” dilution series spanning the EC50 values was used to perform neutralization assays. Each antibody was diluted as indicated in Table 9 below, while the virus concentration was constant. The percent of cells infected (normalized to no antibody control), on epithelial cells, for the various combinations of antibody concentrations is shown in Table 9 below:

TABLE 9

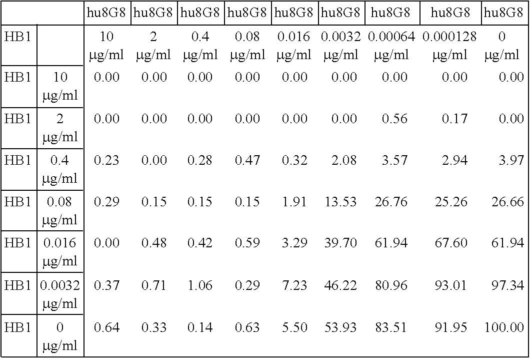

Shaded = concentrations of each antibody in which there is partial neutralization.

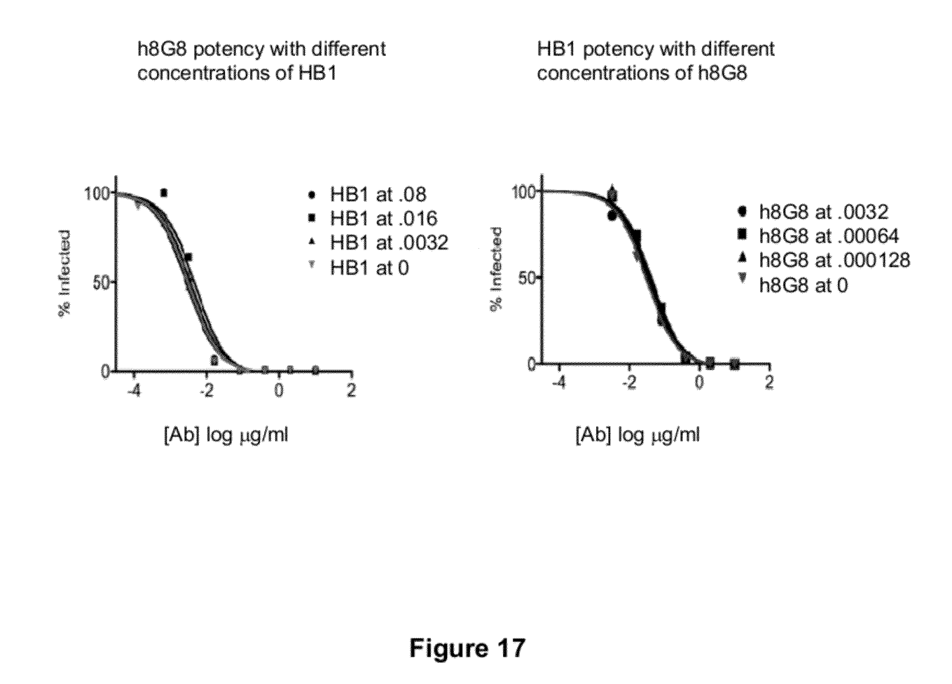

Using neutralization assays, as described above, EC50s for HB1 and hu8G8 were determined in the presence of 0.8, 0.016, 0.0032 μg/ml of the other antibody. The results of these experiments are shown in Tables 10 and 11 below and in FIG. 17.

TABLE 10
hu8G8 potency with different concentrations of HB1

| | EC50 (μg/mL) of |
|---|---|
| Antibody and Concentration | hu8G8 |
| HB1 at .08 μg/ml | 0.0037 |
| HB1 at .016 μg/ml | 0.0047 |
| HB1 at 0032 μg/ml | 0.0028 |
| HB1 at 0 μg/ml | 0.0031 |

TABLE 11
HB1 potency with different concentrations of hu8G8

| | EC50 (μg/mL) of |
|---|---|
| Antibody and Concentration | HB1 |
| hu8G8 at .0032 μg/ml | 0.035 |
| hu8G8 at .00064 μg/ml | 0.043 |
| hu8G8 at .000128 μg/ml | 0.041 |
| hu8G8 at 0 μg/ml | 0.030 |

Upon comparing the potency of each antibody alone with a combination at various ratios, there was no evidence of synergy or antagonism. For example, upon comparing the potency curve of hu8G8 with that of hu8G8 and 0.08 μg/ml of HB1 (infection in the absence of titrated antibody is normalized to 100%), we found that the curves were overlapping (see FIG. 17). In turn, each of the HB1 potency curves overlapped in the presence of various concentrations of hu8G8 (normalized to 100% infection) (see FIG. 17). Thus, at a wide range of ratios, there is no evidence of synergy or antagonism between the antibodies.

### Example 7

### Assessment of Developed Viral Resistance

Although human CMV has a relatively low mutation rate (as compared with hepatitis virus C (HCV) or human immunodeficiency virus (HIV)), the ability of the virus to escape neutralization to HB1 or hu8G8 or both, via the generation of resistant mutations, was assessed. Virus was grown in the presence of sub-optimal concentration of either HB1 (or MSL-109), or hu8G8 or both HB1 and hu8G8 antibodies together. Concentration of each antibody was increased gradually as the virus was passaged onto new cells each week (approximately 50% of the volume was passed forward onto new cells with each round). Mutant viruses resistant to each individual antibody were observed, but no mutant conferred resistance to the combination. All resistant viral mutants emerged from single plaques.

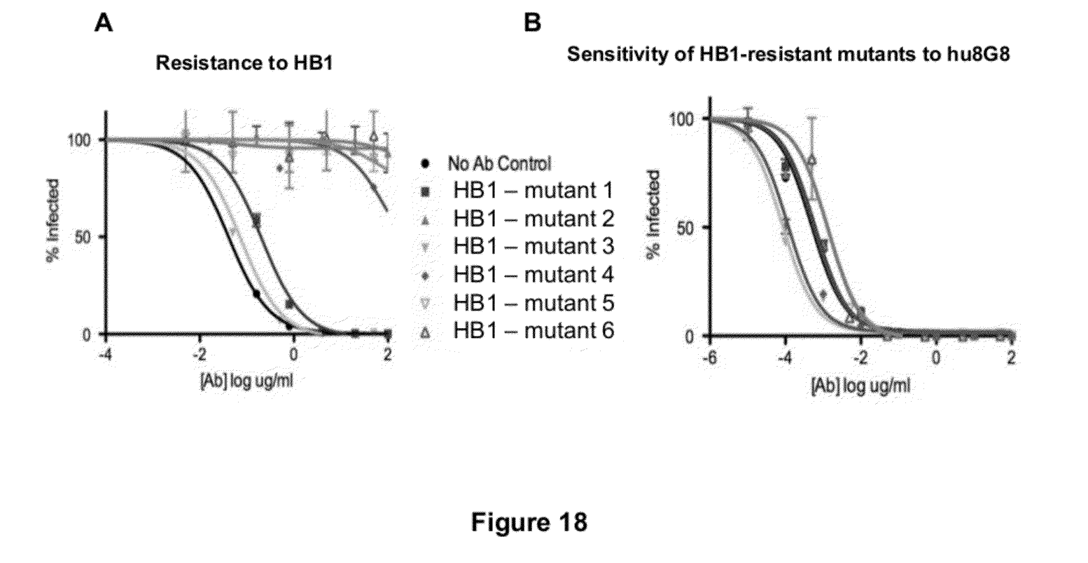

Mutants conferring resistance to neutralization by HB1 emerged (see FIG. 18). However, these mutants were still sensitive to hu8G8 with similar EC50s as shown by neutralization assay (see FIG. 18 and Tables 12 and 13).

TABLE 12
HCMV mutants resistant to HB1 emerged

| | HB1 EC50 (μg/ml) |
|---|---|
| No Ab control | 0.04 |
| HB1 - mutant virus 1 | 0.21 |
| HB1 - mutant virus 2 | No neutralization |
| HB1 - mutant virus 3 | 0.07 |
| HB1 - mutant virus 4 | 200 |
| HB1 - mutant virus 5 | No neutralization |
| HB1 - mutant virus 6 | No neutralization |

TABLE 13
HCMV HB1 resistant mutants are
still sensitive to hu8G8 neutralization

hu8G8 EC50 (μg/ml)

| | Data Set 1 | Data Set 2 |
|---|---|---|
| No Ab control | 0.008 | 0.0005 |
| HB1 - mutant virus 1 | 0.010 | 0.0006 |
| HB1 - mutant virus 2 | 0.008 | 0.006 |
| HB1 - mutant virus 3 | 0.001 | 0.0001 |
| HB1 - mutant virus 4 | Not tested | 0.0001 |
| HB1 - mutant virus 5 | Not tested | 0.001 |
| HB1 - mutant virus 6 | Not tested | 0.001 |

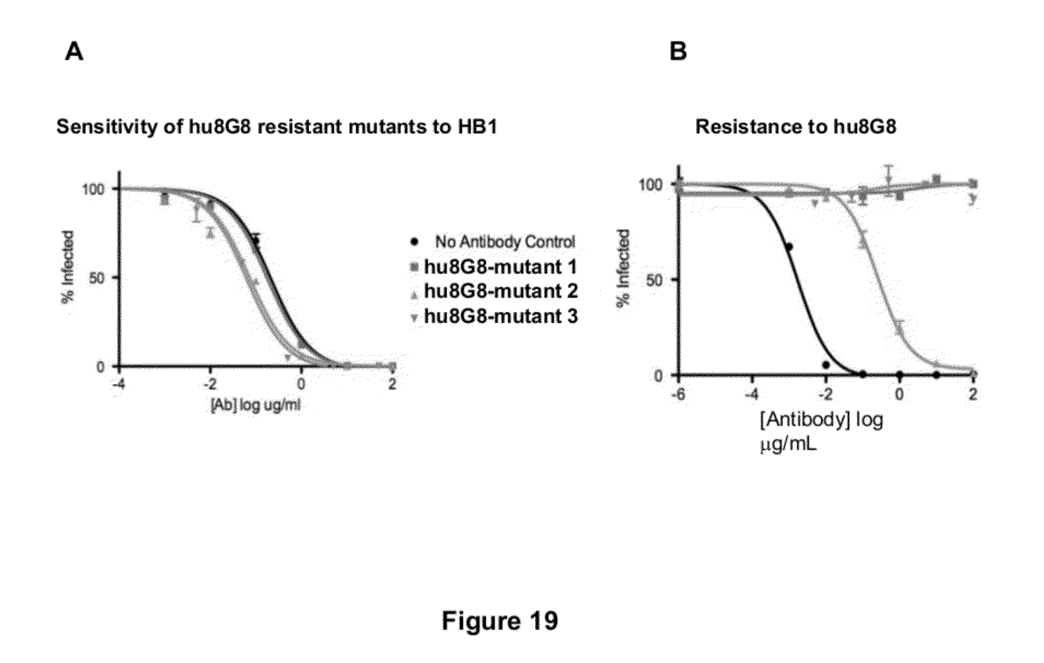

In addition, mutants conferring resistance to neutralization by hu8G8 emerged, and these mutants were still sensitive to HB1 with similar EC50s (see FIG. 19 and Tables 14 and 15).

TABLE 14
HCMV mutants resistant to hu8G8 emerged

| | hu8G8 EC50 (μg/ml) |
|---|---|
| No Ab control | 0.002 |
| hu8G8 - mutant virus 1 | No neutralization |
| hu8G8 - mutant virus 2 | 0.25 |
| hu8G8 - mutant virus 3 | No neutralization |

TABLE 15
HCMV hu8G8 resistant mutants are
still sensitive to HB1 neutralization

| | HB1 EC50 (μg/ml) |
|---|---|
| No Ab control | 0.22 |
| hu8G8 - mutant virus 1 | 0.18 |
| hu8G8 - mutant virus 2 | 0.07 |
| hu8G8 - mutant virus 3 | 0.06 |

To understand the molecular nature of resistance to HB1 and hu8G8 antibodies, gB, gH, gL, UL128, UL130, and UL131 from each resistant strain was sequenced. All strains resistant to HB1 possessed a single non-conservative amino acid mutation in gH, and no other mutations were found in the other glycoproteins, as compared with VR1814 and D1 strain (VR1814 passaged on epithelial cells in parallel without antibody pressure) (Table 16). 11 individual strains resistant to HB1 were generated, encompassing 5 distinct nucleotide mutations in only 3 amino acids. None of these amino acids were found to be mutated in the clinical strains that were sequenced or had published sequences.

Mutations in response to hu8G8 arose less frequently, with only three strains found to be resistant. All three strains resistant to hu8G8 possessed a single non-conservative amino acid mutation in UL131, and no other mutations were found in the other glycoproteins, as compared with VR1814 and D1 strains (see Table 16). When the mutated gH and UL131 sequences were compared to 60 available clinical strain sequences, none of the strains carried these mutations.

TABLE 16
Mapping of Mutations Leading to Resistance

| Resistant strain | Mutated Protein | Residue change |
|---|---|---|
| HB1 - mutant 1 | gH | P171H |
| HB1 - mutant 2 | gH | W168C |
| HB1 - mutant 3 | gH | P171S |
| HB1 - mutant 4 | gH | D446N |
| HB1 - mutant 5 | gH | W168C |
| HB1 - mutant 6 | gH | W168R |
| hu8G8 - mutant 1 | U_{L}131 | Q47K |
| hu8G8 - mutant 2 | U_{L}131 | K51E |
| hu8G8 - mutant 3 | U_{L}131 | D46N |

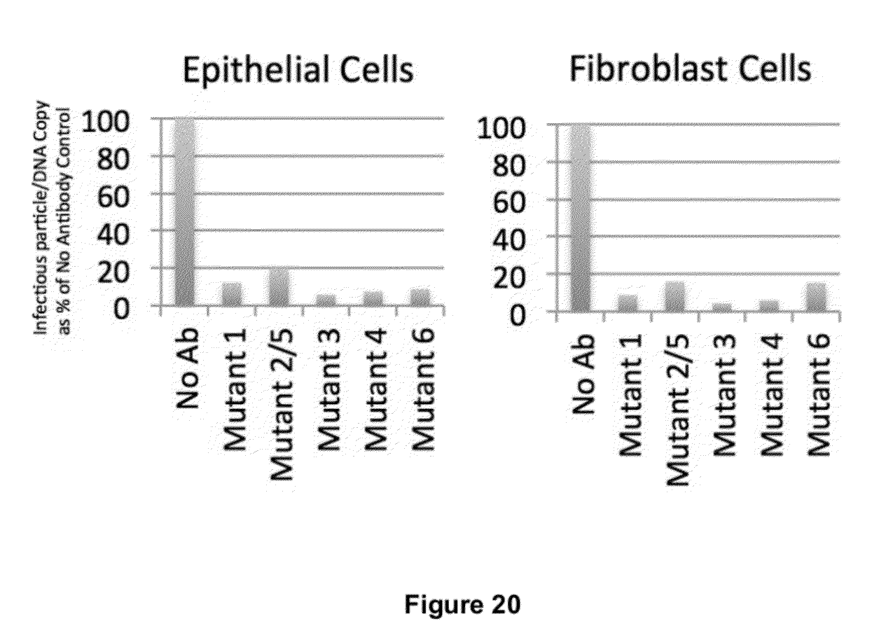

When comparing the ability of HB1-resistant strains to infect cells relative to the D1 strain, these resistant strains had a profound entry defect (up to 20× less efficient), suggesting that these strains would be attenuated for growth in vivo (see FIG. 20). When comparing the ability of hu8G8-resistant strains to infect cells relative to the D1 strain, it was found that the resistant strains could infect cells with equal efficiency; however, these strains are very slow growing, suggesting a defect in production (data not shown). Further analysis is needed to understand the mechanism for this attenuation.

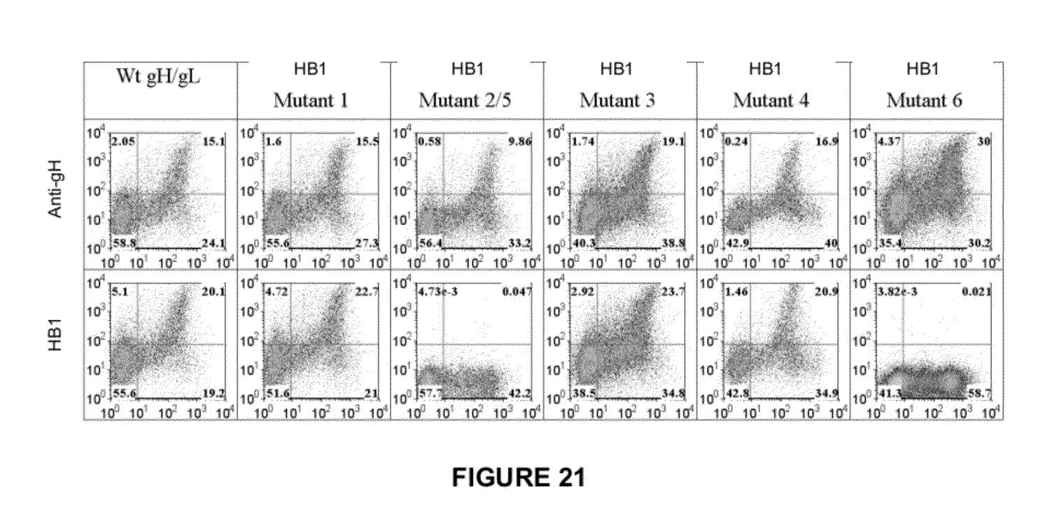

To determine if these mutations affected the ability of HB1 and hu8G8 to bind to gH and UL131, respectively, site-directed mutagenesis was used to transiently express Complex I (gH/gL/UL128/UL130/UL131) with the resistant mutations on the surface of HEK-293T cells and performed FACS analysis of antibody binding. Mutations to P171 (Mutants 1 and 3: P171 to H or S) were only two to five times more resistant to HB1 and binding of this antibody to gH/gL was not noticeably changed. However, the mutations found in the HB1-mutants 2, 5 and 6 (W168 to C or R) completely eliminated the ability of HB1 to bind (see FIG. 21). Furthermore these viral mutants were not neutralized by HB1 (see FIG. 18). Mutant 4 (D446N) displayed an intermediate phenotype; it was 500 times more resistant to neutralization by HB1 (see FIG. 18) but binding to HB1 could still be detected (see FIG. 21).

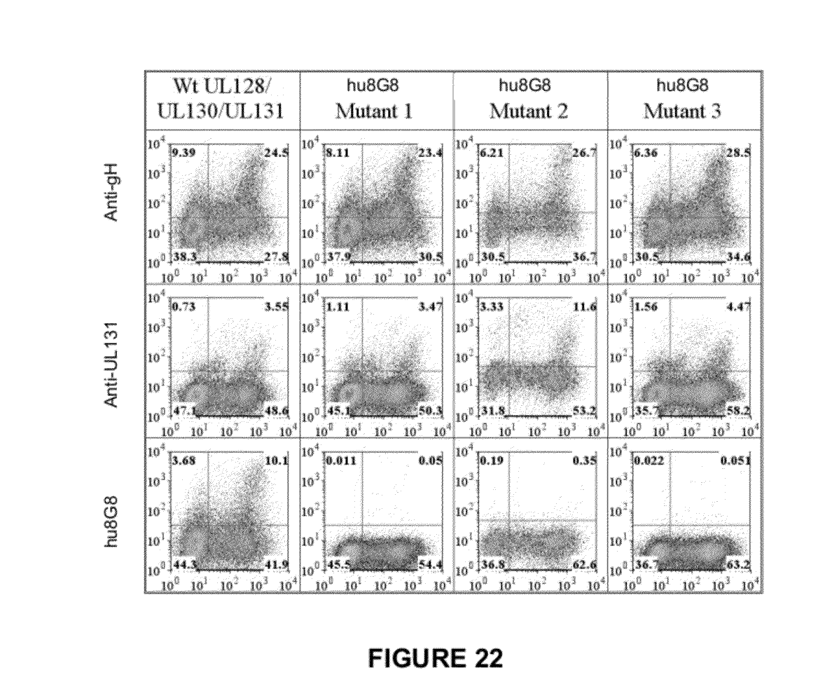

To determine how the mutations in UL131 affected hu8G8 binding, transfections of HEK-293T cells with the wild-type or mutant complex of gH/gL/UL128/UL130/UL131 were performed and binding to anti-gH (HB1 and MSL-109), anti-UL131_{—}993 and hu8G8 was measured by FACS analysis (see FIG. 22). All three UL131 mutations eliminated the binding of hu8G8 for the gH/gL/UL128/UL130/UL131 complex.

The HB1-resistant mutations were mapped onto a model of the structure of HCMV glycoprotein H (based on the recent solved structures for HSV-2 gH and EBV gH) (Backovic et al., PNAS, 197:22635-22640 (2010)). All of the mutated residues mapped to the same face of gH (data not shown). The HB1 Fab was modeled onto the structure of gH. The footprint of the HB1 Fab encompassed all the mutations, suggesting that HB1 binds to the epitope defined by these mutations. Similarly, the hu8G8-resistant mutations are in close proximity (four residues apart) to one another and, although the structure for UL131 is not known, both map to a putative alpha-helical domain and are predicted to lie on the same face of the helix. Thus, together with the binding analysis, the resistance mutations elucidate the epitopes for both HB1 and hu8G8 on the gH/gL/UL128/UL130/UL131 complex.

### Example 8

### Affinity Analysis

The affinity of both HB1 and hu8G8 were determined by biacore and Scatchard analysis, respectively, as described below.

The affinity of HB1 for soluble baculovirus-expressed gH/gL was determined by biacore analysis and was found to be 1 nM. Specifically, the ability of HB1 to bind baculovirus expressed, secreted gH/gL was assessed by surface plasmon resonance (SPR) measurements (Karlsson et al. 1991) using a BIAcore 3000 instrument (GE Healthcare; Piscataway, N.J.). SPR based biosensors report refractive index changes near a surface. When a protein target (“ligand”) is covalently immobilized on the sensor chip surface, SPR can be used to monitor the non covalent interaction of a binding partner (“analyte”) injected over the surface; real time measurements of analyte binding can be used to determine both the kinetics and affinity of the interaction.

In the case of a 1:1 interaction, in which analyte B binds to immobilized ligand A, the equilibrium is described using Equation 1:

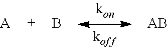

In Equation 1, kₒₙ is the association rate constant, k_{off} is the dissociation rate constant, and the equilibrium dissociation constant K_{D} is determined from K_{D}=k_{off}/kₒₙ. The rate of complex formation for 1:1 binding is determined using Equation 2:

$$\frac{d [ AB ]}{d t} = {k}_{on} [ A ] [ B ] - {k}_{off} [ AB ]$$

When expressed in terms of the SPR signal (R), Equation 2 can be written as Equation 3:

$$\frac{d R}{d t} = {k}_{on} {CR}_{max} - ( {k}_{on} C + {k}_{off} ) R$$

In Equation 3, C is the concentration of free analyte and Rₘₐₓ is the maximum analyte binding capacity of the surface. Similarly, for an analyte B that is capable of dimerization in solution forming 2 binding sites, the equilibrium can be described by equation 4.

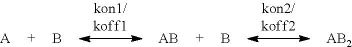

By measuring the concentration dependence of the rate of binding, and the rate of dissociation in the absence of free analyte the kinetic constants can be determined and used to calculate the KD.

SPR measurements were conducted using an anti-Fc capture method to non-covalently immobilize HB1 followed by injection of a varied concentration of gH/gL for determination of the kinetics of binding. A CM5 biosensor chip (BR 1000 14, CM5 research grade; BIAcore, Inc.) was docked, primed with running buffer (10 mM HEPES (pH 7.4), 150 mM NaCl, and 0.01% polysorbate 20) and normalized with 70% glycerol following instructions provided by the manufacturer. As briefly outlined below, a mouse monoclonal anti-human Fc antibody (Human Antibody Capture Kit, BR-1008-39, BIAcore, Inc.) was immobilized on all four flow cells of the CM5 chip. The flow cells were activated using N-ethyl N′ (3 dimethylaminopropyl) carbodiimide hydrochloride and N-hydroxysuccinimide (NHS) (amine coupling kit, BR-1000-50; BIAcore, Inc.) following the protocol described by the manufacturer and using a 7 minute activation time. The activated matrix was then reacted through amine coupling with the capture antibody by injecting 60 μL of 25 μg/mL antibody diluted in 10 mM sodium acetate, pH 5.0, at a flow rate of 10 μL/min. At the end of the coupling injection, any remaining unreacted NHS groups were inactivated by injection of 35 μL of 1 M ethanolamine-HCl at a flow rate of 5 μL/min. The amount of capture antibody covalently immobilized in this way was estimated from the SPR signal before and after the coupling procedure and gave a range of 8000-9500 RU over the 4 flow cells.

An Rₘₐₓ value less than about 100 (arbitrary SPR or Resonance Units (RU)) is commonly accepted as providing a good signal to noise ratio without limiting the range of kinetic constants that can be determined. Preliminary experiments indicated that a 60 μL injection of 0.13 μg/mL HB1 at a flow rate of 30 μL/min resulted in capture of sufficient HB1 such that about 50 RU of signal was observed upon saturation with gH/gL. Thus, this concentration of HB1 and injection protocol was used in the determination of kinetic constants.

Binding measurements were performed by capturing HB1 on flow cell 2 as described above with flow cell 1 used as reference. Solutions of gH/gL varied in concentration from 0.39 nM to 100 nM in 2-fold increments were prepared in running buffer. Sensorgrams were collected for injection of 60 μL of these solutions over the sensor chip surface at a flow rate of 30 μL/min. The sensor chip was maintained at 25° C. and dissociation was monitored for 10 minutes following the end of the injection. The sensor chip surface was regenerated between binding cycles via injection of 30 μL of 3 M MgCl2. This injection caused dissociation of any remaining HB1:gH/gL complex from the capture antibody. HB1 was then captured on flow cell 2 as above for the next binding cycle. A “blank” sensorgram was similarly collected for injection of running buffer over the sensor chip.

The observed sensorgrams were prepared for kinetic analysis by first subtracting the signal measured for the reference cell. Signal resulting from the regeneration portion of the curves was removed. Sensorgrams were then zeroed by subtracting the average RU value of the pre-analyte injection baseline. Finally, the sensorgram measured for injection of running buffer only was subtracted from the curves obtained for injection of solutions containing gH/gL. Data were analyzed according to a 1:1 Langmuir binding model or a bivalent analyte model using software supplied by the manufacturer.

The total SPR signal increased with gH/gL concentration indicating that Fc-captured HB1 was competent for antigen binding. Analysis of the data according to a 1:1 Langmuir binding model indicated an apparent equilibrium dissociation constant (K_{D}) of 0.15 nM with the kinetic constants shown in Table 17; however, the calculated curves did not match well the observed sensorgrams with a relatively large χ² value of 2.1. The observed sensorgrams were better described (χ²=0.4) by the bivalent analyte K_{D} model yielding an apparent K_{D} of 1.0 nM and the kinetic constants shown in Table 18.

TABLE 17
Kinetic constants calculated using 1:1 Langmuir binding model

| Kₒₙ (M⁻¹s⁻¹) | K_{off} (s⁻¹) | Rₘₐₓ (RU) | K_{D} (nM) | χ² |
|---|---|---|---|---|
| 6.8 × 10⁵ | 1.02 × 10⁻⁴ | 42 | 0.15 | 2.1 |

TABLE 18
Kinetic constants calculated using a bivalent analyte KD model

| Kₒₙ | K_{off} | Kₒₙ₂ | K_{off2} | Rₘₐₓ | K_{D} | |
|---|---|---|---|---|---|---|
| (M⁻¹s⁻¹) | (s⁻¹) | (RU⁻¹s⁻¹) | (s⁻¹) | (RU) | (nM) | χ² |
| 2.5 × 10⁵ | 2.5 × 10⁻⁴ | 0.174 | 0.166 | 65 | 1.0 | 0.4 |

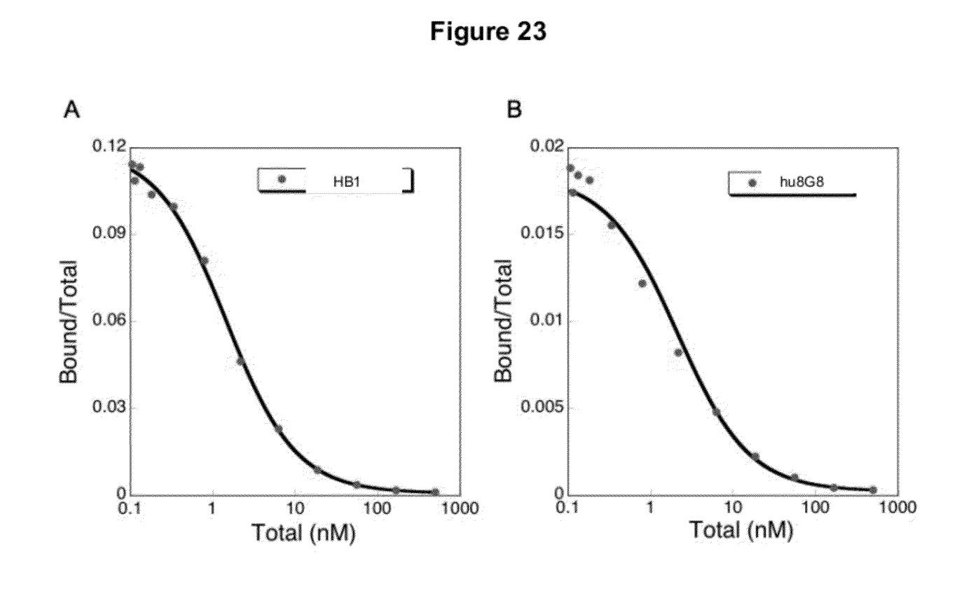

Since biacore could not be used to determine the affinity of hu8G8, Scatchard analysis was employed as an alternative method. In this method, iodinated antibody was mixed with a dilution series of unlabeled antibody and assayed for competition. The results were plotted using the fitting algorithm of Munson and Rodbard to determine affinity of the antibody (FIG. 23). The average Kd is 1.27 nM for HB1 and 2.03 nM for hu8G8 (Table 17). Affinity measurements were also determined by Scatchard analysis on adenovirus cell-surface-expressed gH/gL/UL128/UL130/UL131 complex for both HB1 and hu8G8 and found to be 1.27 nM and 2.03 nM respectively.

Specifically, HB1 and hu8G8 were iodinated using the Iodogen method (Thermo-Fisher Scientific; Waltham, Mass.). The radiolabeled antibodies were purified from free ¹²⁵I—Na by gel filtration using a NAP-5 column. The purified hu8G8 antibody had a specific activity of 12.30 μCi/μg and the purified HB1 antibody had a specific activity of 14.66 μCi/μg. Competition reaction mixtures of 50 μl containing a fixed concentration of iodinated antibody and decreasing concentrations of unlabeled antibody were placed into 96 well plates. The adenoviral transiently transfected ARPE-19 cells expressing the protein complex gH/gL/128/130/131 were detached from flasks using Sigma Accutase® Solution (Sigma-Aldrich; St. Louis, Mo.), were fixed with paraformaldehyde and were washed with binding buffer (DMEM with 2% FBS, 50 mM HEPES, pH 7.2, and 0.1% sodium azide). The washed cells were added at a density of 25,000 cells in 0.2 mL of binding buffer to the 96 well plates containing triplicates of the 50 μL competition reaction mixtures. The final concentration of the iodinated antibody in each competition reaction with cells was 100 pM and the final concentration of the unlabeled antibody in the competition reaction with cells varied, starting at 500 nM and then decreasing by 1:2 fold dilution for 10 concentrations, and included a zero added, buffer only sample. Competition reactions with cells were incubated for 2 hours at room temperature then transferred to a Millipore Multiscreen filter plate and washed four times with binding buffer to separate the free from bound iodinated antibody. The filters were counted on a Wallac Wizard 1470 gamma counter (PerkinElmer Life and Analytical Sciences; Wellesley, Mass.). The binding data were evaluated using New Ligand software (Genentech), which uses the fitting algorithm of Munson and Rodbard (Anal. Biochem., 7:22-39 (1980)) to determine the binding affinity of the antibody.

TABLE 19

| HCMV Antibody | Assay | K_{D} (nM) | ave K_{D} (nM) ± SD^{a} |
|---|---|---|---|
| HB1 | 1 | 1.3 | 1.27 ± 0.06 |
| | 2 | 1.2 | |
| | 3 | 1.3 | |
| Hu8G8 | 1 | 1.96 | 2.03 ± 0.06 |
| | 2 | 2.06 | |
| | 3 | 2.07 | |

^{a}K_{D} = equilibrium dissociation constant; SD = standard deviation

### Example 9

### Analysis of hu8G8 Binding to Complex I

To further characterize the binding of hu8G8 to Complex I, an ELISA assay was performed to test whether hu8G8 could bind a portion of UL131 containing a resistant mutation as identified in Example 7. Specifically the DNA encoding for UL131 was amplified from the codon for serine at position 41 to the codon for serine at position 68 (SRALPDQTRY KYVEQLVDLT LNYHYDAS (SEQ ID NO:194) and cloned into a Restriction-Independent Cloning (RIC) vector with N terminal His6, GST, and a TEV cleavage site (DNA654570). This portion of UL131 forms a putative alpha-helical in the secondary structure of the protein. UL131 was also cloned with the mutation Q47K that eliminates hu8G8 binding to UL131 in the context of Complex I (gH/gL/UL128/UL130/UL131). Sequence verified constructs were grown in E. coli strain Rosetta2 (DE3). Starter cultures were grown over night at 30° C. in LB medium with 50 μg/ml carbenicillin. Protein expression in 1-L cultures was induced at OD₆₀₀ 0.7 with 0.3 mM IPTG at 16° C. overnight. Cells were harvested and immediately lysed by sonication and cell disrupter in 100 mM Tris pH 8.0, 500 mM NaCl, 5% glycerol (Buffer A) containing EDTA-free protease inhibitor tablets (Roche). Lysed cells were spun down at 10000 rpm for 40 min and the cleared lysates loaded onto a gravity flow Ni-chelating affinity column (Qiagen). Columns were washed with 10 column volumes Buffer A and 10 column volumes Buffer A with 50 mM imidazole. Proteins were eluted with 100 mM Tris pH 8.0, 500 mM NaCl, 5% glycerol, 500 mM imidazole and immediately dialyzed into 50 mM Tris pH 8.0, 200 mM NaCl, and 5% glycerol. Proteins were further purified on a size exclusion chromatography column (S200 10/30, GE) in 25 mM Tris pH 8.0, 200 mM NaCl and 5% glycerol.

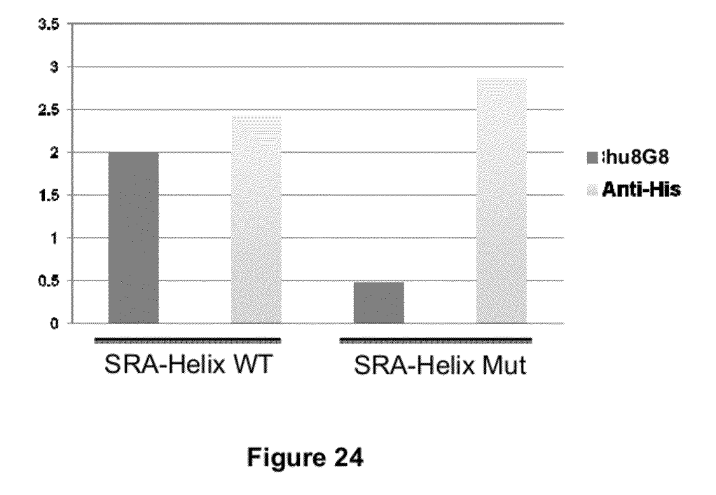

To determine hu8G8 binding to the UL131 protein fragments, Maxsorb ELISA plates were coated overnight at 4° C. with 1 μg, 200 ng, or 40 ng protein per well in carbonate coating buffer. After 3 washes with wash buffer (PBS with 0.05% Tween 20 [Sigma Chemical]), wells were blocked for an hour with assay diluent (wash buffer with 0.5% BSA [Invitrogen; Carlsbad, Calif.]). hu8G8 was incubated at 10 μg/ml or 1 μg/ml for an hour in assay diluent. After 3 washes, wells were either incubated with peroxidase-conjugated anti-human antibody (Jackson Immunolabs, Bar Harbor, Me.) at 1:5000 or horseradish peroxidase-conjugated anti-penta-HIS (Qiagen) incubated at 1:500 or 1:5000 for 1 hour. The results of the experiment are shown in FIG. 24 and include data from the ELISA plates coated with 200 ng protein and incubated with 10 μg/ml hu8G8.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1. An isolated antibody that binds HCMV Complex I comprising three heavy chain hypervariable regions (HVR-H1, HVR-H2 and HVR-H3) and three light chain hypervariable regions (HVR-L1, HVR-L2 and HVR-L3), wherein:
(a) HVR-H1 comprises the amino acid sequence of SEQ ID NO:6;
(b) HVR-H2 comprises the amino acid sequence of SEQ ID NO:7;
(c) HVR-H3 comprises the amino acid sequence of SEQ ID NO:8;
(d) HVR-L1 comprises the amino acid sequence of SEQ ID NO:9;
(e) HVR-L2 comprises an amino acid sequence selected from SEQ ID NOs:10-19; and
(f) HVR-L3 comprises the amino acid sequence of SEQ ID NO:20.

2. An isolated antibody that binds HCMV Complex I comprising three heavy chain hypervariable regions (HVR-H1, HVR-H2 and HVR-H3) and three light chain hypervariable regions (HVR-L1, HVR-L2 and HVR-L3), wherein:
(a) HVR-H1 comprises the amino acid sequence of SEQ ID NO:6;
(b) HVR-H2 comprises the amino acid sequence of SEQ ID NO:7;
(c) HVR-H3 comprises the amino acid sequence of SEQ ID NO:8;
(d) HVR-L1 comprises the amino acid sequence of SEQ ID NO:9;
(f) HVR-L3 comprises the amino acid sequence of SEQ ID NO:20; and
(e) HVR-L2 and the first amino acid of the light chain variable domain framework FR3 comprises the amino acid sequence of SEQ ID NO:21.

3. The antibody of claim 1, wherein said antibody that binds HCMV Complex I comprises a light chain variable domain framework FR1 comprising an amino acid sequence selected from SEQ ID NO:35, SEQ ID NO:39, and SEQ ID NO:43; and a light chain variable domain framework FR2 comprising an amino acid sequence selected from SEQ ID NO:36, SEQ ID NO:40, and SEQ ID NO:44.

4. The antibody of claim 1, wherein said antibody that binds HCMV Complex I comprises a light chain variable domain framework FR3 comprising an amino acid sequence selected from SEQ ID NO:37 and SEQ ID NO:41; and a light chain variable domain framework FR4 comprising the amino acid sequence selected from SEQ ID NO:38 and SEQ ID NO:42.

5. The antibody of claim 1, wherein said antibody that binds HCMV Complex I comprises a V_{H} sequence having at least 95% sequence identity to the amino acid sequence selected from SEQ ID NO:45, SEQ ID NO:46 and SEQ ID NO:47 and a V_{L} sequence having at least 95% sequence identity to the amino acid sequence of SEQ ID NO:48 or SEQ ID NO:49.

6. The antibody of claim 5, wherein said V_{H} sequence comprises the amino acid sequence selected from SEQ ID NO:45, SEQ ID NO:46 and SEQ ID NO:47.

7. The antibody of claim 5, wherein said V_{L} sequence comprises the amino acid sequence of SEQ ID NO:48 or SEQ ID NO:49.

8. The antibody of claim 5, wherein said antibody that binds HCMV Complex I comprises a V_{H} comprising the amino acid sequence selected from SEQ ID NO:45, SEQ ID NO:46 or SEQ ID NO:47; and a V_{L} comprising the amino acid sequence of SEQ ID NO:48 or SEQ ID NO:49.

9. The antibody of claim 8, wherein said antibody that binds HCMV Complex I comprises a V_{H} sequence of SEQ ID NO:45 or SEQ ID NO:46 and a V_{L} sequence of SEQ ID NO:49.

10. The antibody of any one of claims 1-9, wherein the antibody that binds to HCMV Complex I neutralizes 50% of HCMV at an antibody concentration of 0.05 μg/ml to 0.0007 μg/ml or less.

11. An isolated antibody that binds HCMV gH comprising three heavy chain hypervariable regions (HVR-H1, HVR-H2 and HVR-H3) and three light chain hypervariable regions (HVR-L1, HVR-L2 and HVR-L3), wherein:
(a) HVR-H1 comprises the amino acid sequence of SEQ ID NO:71;
(b) HVR-H2 comprises an amino acid sequence selected from SEQ ID NO:72, SEQ ID NO:73, SEQ ID NO:74, and SEQ ID NO:93;
(c) HVR-H3 comprises the amino acid sequence of SEQ ID NO:75;
(d) HVR-L1 comprises the amino acid sequence of SEQ ID NO:76;
(e) HVR-L2 comprises the amino acid sequence of SEQ ID NO:77; and
(f) HVR-L3 comprises the amino acid sequence of SEQ ID NO:78.

12. The antibody of claim 11, wherein the antibody that binds HCMV gH comprises an HVR-H2 comprising the amino acid sequence of SEQ ID NO:93, wherein the amino acid at position 6 of SEQ ID NO:93 is selected from the group consisting of Ser, Thr, Asn, Gln, Phe, Met, and Leu, and the amino acid at position 8 of SEQ ID NO:93 is selected from the group consisting of Thr and Arg.

13. The antibody of claim 12, wherein the antibody that binds HCMV gH comprises an HVR-H2 comprising an amino acid sequence selected from SEQ ID NO:72, SEQ ID NO:73 and SEQ ID NO:74.

14. The antibody of claim 13, wherein HVR-H2 comprises the amino acid sequence of SEQ ID NO:74.

15. The antibody of claim 12, wherein the antibody that binds HCMV gH comprises a VH sequence having at least 95% sequence identity to the amino acid sequence of SEQ ID NO:94, wherein the amino acid at position 54 of SEQ ID NO:94 is selected from the group consisting of Ser, Thr, Asn, Gln, Phe, Met, and Leu and the amino acid at position 56 of SEQ ID NO:94 is selected from Thr or Arg.

16. The antibody of claim 15, wherein the VH comprises an amino acid sequence selected from the group consisting of: SEQ ID NO:87, SEQ ID NO:88 and SEQ ID NO:89.

17. The antibody of claim 15, wherein the antibody that binds HCMV gH comprises a VL sequence having at least 95% sequence identity to the amino acid sequence of SEQ ID NO:90.

18. The antibody of claim 17, wherein the antibody that binds HCMV gH comprises a VL sequence of SEQ ID NO:90.

19. The antibody of claim 18, wherein the antibody that binds HCMV gH comprises a VH sequence of SEQ ID NO:89.

20. The antibody of any one of claims 11-19, wherein the antibody that binds to HCMV gH neutralizes HCMV at an EC90 of 0.001 to 0.01 μg/ml.

21. The antibody of any one of claims 1-20, wherein said antibody is a monoclonal antibody.

22. The antibody of any one of claims 1-21, which is a human, humanized or chimeric antibody.

23. The antibody of any one of claims 1-22, wherein said antibody is an antibody fragment.

24. The antibody of any one of claims 1-10 and 21-22, wherein the antibody which binds HCMV Complex I is a full length IgG1 antibody.

25. The antibody of any one of claims 11-22, wherein the antibody which binds HCMV gH is a full length IgG1 antibody.

26. A composition comprising an antibody of any one of claims 1-25.

27. The composition of claim 26 further comprising an additional therapeutic agent.

28. The composition of claim 26 or 27 further comprising a pharmaceutically acceptable carrier.

29. An isolated nucleic acid encoding the antibody of any one of claims 1-25.

30. A host cell comprising the nucleic acid of claim 29.

31. A method of producing an antibody comprising culturing the host cell of claim 30 so that an antibody is produced.

32. A composition comprising an isolated antibody that binds HCMV Complex I and an isolated antibody that binds HCMV gH.

33. The composition of claim 32, wherein the composition neutralizes HCMV infection.

34. The composition of claim 33, wherein the composition neutralizes at least 50% of HCMV.

35. The composition of any one of claims 32-34, wherein the antibody that binds HCMV Complex I and the antibody that binds HCMV gH are present in the composition in a 1:1 ratio.

36. The composition of any one of claims 32-35, wherein the concentration of the antibody that binds to HCMV Complex I is at least 0.0007 μg/ml to 0.05 μg/ml and the concentration of the antibody that binds to HCMV gH is at least 0.001 to 0.01 μg/ml.

37. The composition of any one of claims 32-36, wherein the antibody that binds HCMV Complex I comprises three heavy chain hypervariable regions (HVR-H1, HVR-H2 and HVR-H3) and three light chain hypervariable regions (HVR-L1, HVR-L2 and HVR-L3), wherein:
(a) HVR-H1 comprises the amino acid sequence of SEQ ID NO:6;
(b) HVR-H2 comprises the amino acid sequence of SEQ ID NO:7;
(c) HVR-H3 comprises the amino acid sequence of SEQ ID NO:8;
(d) HVR-L1 comprises the amino acid sequence of SEQ ID NO:9;
(e) HVR-L2 comprises an amino acid sequence selected from SEQ ID NOs:10-19; and
(f) HVR-L3 comprises the amino acid sequence of SEQ ID NO:20.

38. The composition of any one of claims 32-36, wherein said antibody that binds HCMV gH comprises three heavy chain hypervariable regions (HVR-H1, HVR-H2 and HVR-H3) and three light chain hypervariable regions (HVR-L1, HVR-L2 and HVR-L3), wherein:
(a) HVR-H1 comprises the amino acid sequence of SEQ ID NO:71;
(b) HVR-H2 comprises an amino acid sequence selected from SEQ ID NO:72, SEQ ID NO:73, SEQ ID NO:74, and SEQ ID NO:93;
(c) HVR-H3 comprises the amino acid sequence of SEQ ID NO:75;
(d) HVR-L1 comprises the amino acid sequence of SEQ ID NO:76;
(e) HVR-L2 comprises the amino acid sequence of SEQ ID NO:77; and
(f) HVR-L3 comprises the amino acid sequence of SEQ ID NO:78.

39. The composition of any one of claims 32-38 wherein said antibody that binds HCMV Complex I comprises a light chain variable domain framework FR1 comprising an amino acid sequence selected from SEQ ID NO:35, SEQ ID NO:39, and SEQ ID NO:43; and a light chain variable domain framework FR2 comprising an amino acid sequence selected from SEQ ID NO:36, SEQ ID NO:40, and SEQ ID NO:44.

40. The composition of any one of any one of claims 32-39 wherein said antibody that binds HCMV Complex I comprises a light chain variable domain framework FR3 comprising an amino acid sequence selected from SEQ ID NO:37 and SEQ ID NO:41; and a light chain variable domain framework FR4 comprising the amino acid sequence selected from SEQ ID NO:38 and SEQ ID NO:42.

41. The composition of any one of claims 32-40 wherein said antibody that binds HCMV Complex I comprises a V_{H} sequence having at least 95% sequence identity to the amino acid sequence selected from SEQ ID NO:45, SEQ ID NO:46 and SEQ ID NO:47 and a V_{L} sequence having at least 95% sequence identity to the amino acid sequence of SEQ ID NO:48 or SEQ ID NO:49.

42. The composition of claim 41, wherein said V_{H} sequence comprises the amino acid sequence selected from SEQ ID NO:45, SEQ ID NO:46 and SEQ ID NO:47.

43. The composition of claim 41, wherein said V_{L} sequence comprises the amino acid sequence of SEQ ID NO:48 or SEQ ID NO:49.

44. The composition of claim 41, wherein said antibody that binds HCMV Complex I comprises (a) a V_{H} comprising the amino acid sequence selected from SEQ ID NO:45, SEQ ID NO:46 or SEQ ID NO:47; and (b) a V_{L} comprising the amino acid sequence of SEQ ID NO:48 or SEQ ID NO:49.

45. The composition of claim 44, wherein said antibody that binds HCMV Complex I comprises a V_{H} sequence of SEQ ID NO:45 or SEQ ID NO:46 and a V_{L} sequence of SEQ ID NO:49.

46. The composition of any one of claims 32-45, wherein the antibody that binds HCMV gH comprises an HVR-H2 comprising the amino acid sequence of SEQ ID NO:93, wherein the amino acid at position 6 of SEQ ID NO:93 is selected from the group consisting of Ser, Thr, Asn, Gln, Phe, Met, and Leu, and the amino acid at position 8 of SEQ ID NO:93 is selected from the group consisting of Thr and Arg.

47. The composition of any one of claims 32-46, wherein the antibody that binds HCMV gH comprises an HVR-H2 comprising an amino acid sequence selected from SEQ ID NO:72, SEQ ID NO:73 and SEQ ID NO:74.

48. The composition of claim 47, wherein HVR-H2 comprises the amino acid sequence of SEQ ID NO:74.

49. The composition of any one of claims 32-46, wherein the antibody that binds HCMV gH comprises a VH sequence having at least 95% sequence identity to the amino acid sequence of SEQ ID NO:94, wherein the amino acid at position 54 of SEQ ID NO:94 is selected from the group consisting of Ser, Thr, Asn, Gln, Phe, Met, and Leu and the amino acid at position 56 of SEQ ID NO:94 is selected from Thr or Arg.

50. The composition of claim 49, wherein the VH comprises an amino acid sequence selected from the group consisting of: SEQ ID NO:87, SEQ ID NO:88 and SEQ ID NO:89.

51. The composition of claim 50, wherein the antibody that binds HCMV gH comprises comprising a VL sequence having at least 95% sequence identity to the amino acid sequence of SEQ ID NO:90.

52. The composition of claim 51, wherein the antibody that binds HCMV gH comprises a VL sequence of SEQ ID NO:90.

53. The composition of claim 52, wherein the antibody that binds HCMV gH comprises a VH sequence of SEQ ID NO:89.

54. The composition of any one of claims 32-53, wherein said antibody that binds HCMV Complex I and said antibody that binds HCMV gH are monoclonal antibodies.

55. The composition of any one of claims 32-54, wherein said antibody that binds HCMV Complex I and said antibody that binds HCMV gH are human, humanized, or chimeric antibodies.

56. The composition of any one of claims 32-55, wherein said antibody that binds HCMV Complex I is an antibody fragment.

57. The composition of any one of claims 32-55, wherein said antibody that binds HCMV gH is an antibody fragment.

58. The composition of any one of claims 32-55 and 57, wherein the antibody which binds HCMV Complex I is a full length IgG1 antibody.

59. The composition of any one of claims 32-55 and 56, wherein the antibody which binds HCMV gH is a full length IgG1 antibody.

60. The composition of any one of claims 32-59, further comprising a pharmaceutically acceptable carrier.

61. The composition of claim 60, further comprising an additional therapeutic agent.

62. The composition of claim 61, wherein the additional therapeutic agent is selected from ganciclovir, foscarnet, valganciclovir and cidofovir.

63. The composition of any one of claims 26-28 and 32-62 for use as a medicament.

64. The composition of any one of claims 26-28 and 32-62 for use in inhibiting, treating or preventing HCMV infection.

65. The composition of any one of claims 26-28 and 32-62 for use in inhibiting, treating or preventing congenital HCMV infection or HCMV infection in a transplant recipient.

66. The composition of any one of claims 63-65, wherein the antibody which binds HCMV gH is in a composition separate from the antibody which binds HCMV Complex I.

67. Use of the composition of any one of claims 26-28 and 32-62 in the manufacture of a medicament.

68. The use of claim 67, wherein the medicament is for treatment, inhibition or prevention of HCMV infection.

69. The use of claim 68, wherein the medicament is for inhibiting, preventing or treating congenital HCMV infection or HCMV infection in a transplant recipient.

70. The use of any one of claims 67-69 wherein the medicament comprises the antibody that binds HCMV Complex I in a composition separate from the antibody that binds HCMV gH.

71. A method of treating, inhibiting or preventing HCMV infection comprising administering to a patient an effective amount of the composition of any one of claims 26-28 and 32-62.

72. A method of treating, inhibiting or preventing congenital HCMV infection comprising administering to a pregnant patient an effective amount of the composition of any one of claims 26-28 and 32-62.

73. A method of treating, inhibiting or preventing HCMV infection in a transplant recipient comprising administering to the transplant recipient an effective amount of the composition of any one of claims 26-28 and 32-62 to treat, inhibit or prevent HCMV infection.

74. The method of claim 73, wherein the transplant recipient is HCMV seronegative.

75. The method of claim 74, wherein the transplant recipient is receiving or has received an organ or tissue from a HCMV seropositive donor.

76. The method of any one of claims 71-75, further comprising administering an additional therapeutic agent to the patient.

77. The method of any one of claims 71-76 wherein the composition comprising the antibody which binds HCMV Complex I is administered separately from the composition comprising the antibody which bind HCMV gH.

78. The method of any one of claims 71-77, wherein the composition comprising the antibody which binds HCMV gH is a composition separate from the composition comprising the antibody which binds HCMV Complex I.

79. The method of claim 77 or 78 wherein the composition comprising the antibody which binds HCMV gH is administered simultaneously with the composition comprising the antibody which binds HCMV Complex I.

80. The method of claim 77 or 78 wherein the composition comprising the antibody which binds HCMV gH is administered prior to or subsequent to the composition comprising the antibody which binds HCMV Complex I.

81. Use of an antibody or a combination of antibodies of any one of claims 1-25 in the manufacture of a medicament.

82. The use of claim 81, wherein the medicament is for inhibition, prevention or treatment of HCMV infection.

83. The use of claim 82, wherein the medicament is for inhibiting, preventing or treating congenital HCMV infection or HCMV infection in a transplant recipient.

84. Use of an antibody or combination of antibodies, of any one of claims 1-25 for use in treating, preventing or inhibiting HCMV infection.

85. The use of claim 84, wherein the treatment is to prevent or inhibit congenital HCMV infection or HCMV infection in a transplant recipient.

86. A method of treating, preventing or inhibiting HCMV infection comprising administering an effective amount of an antibody or a combination of antibodies of any one of claims 1-25 to a patient.

87. A method of treating, preventing or inhibiting congenital HCMV infection comprising administering to a pregnant patient an effective amount of an antibody or a combination of antibodies of any one of claims 1-25.

88. A method of treating, preventing or inhibiting HCMV infection in a transplant recipient comprising administering to the transplant recipient an effective amount of an antibody or combination of antibodies of any one of claims 1-25.

89. The method of claim 88, wherein the transplant recipient is HCMV seronegative.

90. The method of claim 89, wherein the transplant recipient is receiving or has received an organ or tissue from a HCMV seropositive donor.

91. The method of claim 90, further comprising administering an additional therapeutic agent to the patient.

92. The method of any one of claims 86-91 wherein the antibody which binds HCMV Complex I is administered separately from the antibody which bind HCMV gH.

93. The method of claim 92, wherein the antibody which binds HCMV gH is administered simultaneously with the antibody which binds HCMV Complex I.

94. The method of claim 92, wherein the antibody which binds HCMV gH is administered prior to or subsequent to the antibody which binds HCMV Complex I.

95. An isolated antibody which binds to the same epitope as any one of the antibodies of claims 1-25.

96. An isolated antibody which binds to an epitope of HCMV gH comprising amino acids which correspond to the amino acids selected from the group consisting of:
(i) tryptophan at position 168 of SEQ ID NO:1;
(ii) aspartic acid at position 446 of SEQ ID NO:1;
(iii) proline at position 171 of SEQ ID NO:1; and
(iv) combinations thereof.

97. The antibody of claim 96, which binds to an epitope of HCMV gH comprising amino acids selected from the group consisting of:
(i) tryptophan at position 168 of SEQ ID NO:1;
(ii) aspartic acid at position 446 of SEQ ID NO:1;
(iii) proline at position 171 of SEQ ID NO:1; and
(iv) combinations thereof.

98. An isolated antibody which binds to an epitope of HCMV Complex I comprising amino acids which correspond to the amino acids selected from the group consisting of:
(i) glutamine at position 47 of SEQ ID NO:203;
(ii) lysine at position 51 of SEQ ID NO:203;
(iii) aspartic acid at position 46 of SEQ ID NO: 203; and
(iv) combinations thereof.

99. The antibody of claim 98 comprising amino acids selected from the group consisting of:
(i) glutamine at position 47 of SEQ ID NO:203;
(ii) lysine at position 51 of SEQ ID NO: 203;
(iii) aspartic acid at position 46 of SEQ ID NO:203; and
(iv) combinations thereof.

100. An isolated antibody which binds to a polypeptide of HCMV Complex I, comprising the amino acid sequence SRALPDQTRYKYVEQLVDLT LNYHYDAS (SEQ ID NO:194).

101. A method of reducing or preventing an increase in HCMV viral titer in a patient comprising administering to the patient an effective amount of an antibody or combination of antibodies of any one of claims 1-25.

102. A method of reducing or preventing an increase in HCMV viral titer in a patient comprising administering to the patient an effective amount of the composition of any one of claims 26-28 and 32-62.


SEQUENCE LISTING (209 sequences)

SEQ ID NO: 1 - protein, 743 aa, Human cytomegalovirus
MRPGLPPYLTVFTVYLLSHLPSQRYGADAASEALDPHAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFSQQLKAQDSLGQQPTTVPPPIDLSIPHVWMPPQTTPHDWKGSHTTSGLHRPHFNQTCILFDGHDLLFSTVTPCLHQGFYLMDELRYVKITLTEDFFVVTVSIDDDTPMLLIFGHLPRVLFKAPYQRDNFILRQTEKHELLVLVKKAQLNRHSYLKDSDFLDAALDFNYLDLSALLRNSFHRYAVDVLKSGRCQMLDRRTVEMAFAYALALFAAARQEEAGTEISIPRALDRQAALLQIQEFMITCLSQTPPRTTLLLYPTAVDLAKRALWTPDQITDITSLVRLVYILSKQNQQHLIPQWALRQIADFALQLHKTHLASFLSAFARQELYLMGSLVHSMLVHTTERREIFIVETGLCSLAELSHFTQLLAHPHHEYLSDLYTPCSSSGRRDHSLERLTRLFPDATVPATVPAALSILSTMQPSTLETFPDLFCLPLGESFSALTVSEHVSYVVTNQYLIKGISYPVSTTVVGQSLIITQTDSQTKCELTRNMHTTHSITAALNISLENCAFCQSALLEYDDTQGVINIMYMHDSDDVLFALDPYNEVVVSSPRTHYLMLLKNGTVLEVTDVVVDATDSRLLMMSVYALSAIIGIYLLYRMLKTC

SEQ ID NO: 2 - protein, 278 aa, Human cytomegalovirus
MCRRPDCGFSFSPGPVVLLWCCLLLPIVSSVAVSVAPTAAEKVPAECPELTRRCLLGEVFQGDKYESWLRPLVNVTRRDGPLSQLIRYRPVTPEAANSVLLDDAFLDTLALLYNNPDQLRALLTLLSSDTAPRWMTVMRGYSECGDGSPAVYTCVDDLCRGYDLTRLSYGRSIFTEHVLGFELVPPSLFNVVVAIRNEATRTNRAVRLPVSTAAAPEGITLFYGLYNAVKEFCLRHQLDPPLLRHLDKYYAGLPPELKQTRVNLPAHSRYGPQAVDAR

SEQ ID NO: 3 - protein, 171 aa, Human cytomegalovirus
MSPKNLTPFLTALWLLLGHSRVPRVRAEECCEFINVNHPPERCYDFKMCNRFTVALRCPDGEVCYSPEKTAEIRGIVTTMTHSLTRQVVHNKLTSCNYNPLYLEADGRIRCGKVNDKAQYLLGAAGSVPYRWINLEYDKITRIVGLDQYLESVKKHKRLDVCRAKMGYMLQ

SEQ ID NO: 4 - protein, 214 aa, Human cytomegalovirus
MLRLLLRHHFHCLLLCAVWATPCLASPWSTLTANQNPSPPWSKLTYSKPHDAATFYCPFLYPSPPRSPLQFSGFQRVSTGPECRNETLYLLYNREGQTLVEKSSTWVKKVIWYLSGRNQTILQRMPRTASKPSDGNVQISVEDAKIFGAHMVPKQTKLLRFVVNDGTRYQMCVMKLESWAHVFRDYSVSFQVRLTFTEANNQTYTFCTHPNLIV

SEQ ID NO: 5 - protein, 76 aa, Human cytomegalovirus
MCMMSHNKAFFLSLQHAAVSGVAVCLSVRRGAGSVPAGNRGKKTIITEYRITGTRALARCPTKPVTSMWNSSWTSR

SEQ ID NO: 6 - protein, 10 aa, Mus musculus
GYTFTNYGMN

SEQ ID NO: 7 - protein, 17 aa, Mus musculus
WINTYTGEPTYADDFKG

SEQ ID NO: 8 - protein, 13 aa, Mus musculus
SWYYVSNYWYFDV

SEQ ID NO: 9 - protein, 12 aa, Mus musculus
TLSSQHSTYTIE

SEQ ID NO: 10 - protein, 11 aa, Mus musculus
LKKDGSHSTGD

SEQ ID NO: 11 - protein, 11 aa, Artificial Sequence
  Description of Artificial Sequence Synthetic Mutated mouse HVR-L2 peptide
LKKDASHSTGD

SEQ ID NO: 12 - protein, 11 aa, Artificial Sequence
  Description of Artificial Sequence Synthetic Mutated mouse HVR-L2 peptide
LKKEGSHSTGD

SEQ ID NO: 13 - protein, 11 aa, Artificial Sequence
  Description of Artificial Sequence Synthetic Mutated mouse HVR-L2 peptide
LKKTGSHSTGD

SEQ ID NO: 14 - protein, 11 aa, Artificial Sequence
  Description of Artificial Sequence Synthetic Mutated mouse HVR-L2 peptide
LKKDGSHSTGD

SEQ ID NO: 15 - protein, 11 aa, Artificial Sequence
  Description of Artificial Sequence Synthetic Mutated mouse HVR-L2 peptide
LKKDGSHSTGE

SEQ ID NO: 16 - protein, 11 aa, Artificial Sequence
  Description of Artificial Sequence Synthetic Mutated mouse HVR-L2 peptide
LKKDGSHSTGT

SEQ ID NO: 17 - protein, 11 aa, Artificial Sequence
  Description of Artificial Sequence Synthetic Mutated mouse HVR-L2 peptide
LKKSGSHSTGD

SEQ ID NO: 18 - protein, 11 aa, Artificial Sequence
  Description of Artificial Sequence Synthetic Mutated mouse HVR-L2 peptide
LKKSGSHSTGS

SEQ ID NO: 19 - protein, 11 aa, Artificial Sequence
  Description of Artificial Sequence Synthetic Mutated mouse HVR-L2 peptide
LKKTGSHSTGD

SEQ ID NO: 20 - protein, 13 aa, Mus musculus
GVGDTIKEQFVYV

SEQ ID NO: 21 - protein, 12 aa, Artificial Sequence
  Description of Artificial Sequence Synthetic Consensus HVR-L2 Sequence
LKKXXSHSTGXX

SEQ ID NO: 22 - protein, 25 aa, Homo sapiens
EVQLVQSGAEVKKPGASVKVSCKAS

SEQ ID NO: 23 - protein, 14 aa, Homo sapiens
WVRQAPGQGLEWIG

SEQ ID NO: 24 - protein, 32 aa, Homo sapiens
RVTITRDTSTSTAYLELSSLRSEDTAVYYCAR

SEQ ID NO: 25 - protein, 11 aa, Homo sapiens
WGQGTLVTVSS

SEQ ID NO: 26 - protein, 25 aa, Mus musculus
QIHLVQSGPELKKPGETVKISCKAS

SEQ ID NO: 27 - protein, 14 aa, Mus musculus
WVKQAPGKGLKWMG

SEQ ID NO: 28 - protein, 32 aa, Mus musculus
RFAFSLETSASTAYLQINNLKNEDMATYFCAR

SEQ ID NO: 29 - protein, 11 aa, Mus musculus
WGQGTLVTVSS

SEQ ID NO: 30 - protein, 25 aa, Homo sapiens [sequence omitted]

SEQ ID NO: 31 - protein, 14 aa, Homo sapiens
WVRQAPGKGLEWVG

SEQ ID NO: 32 - protein, 32 aa, Homo sapiens
RFTISRDNSKNTLYLQMNSLRAEDTAVYYCAR

SEQ ID NO: 33 - protein, 25 aa, Homo sapiens
EVQLVQSGSELKKPGASVKVSCKAS

SEQ ID NO: 34 - protein, 32 aa, Homo sapiens
RFVFSLDTSVSTAYLQISSLKAEDTAVYYCAR

SEQ ID NO: 35 - protein, 22 aa, Mus musculus
QLVLTQSSSASFSLGASAKLTC

SEQ ID NO: 36 - protein, 15 aa, Mus musculus
WYQQQPLKPPKYVME

SEQ ID NO: 37 - protein, 32 aa, Mus musculus
GIPDRFSGSSSGADRYLSISNIQPEDEAIYIC

SEQ ID NO: 38 - protein, 11 aa, Mus musculus
FGGGTKVTVLG

SEQ ID NO: 39 - protein, 22 aa, Homo sapiens
QPVLTQSPSASASLGASVKLTC

SEQ ID NO: 40 - protein, 15 aa, Homo sapiens
WHQQQPGKGPRYLMK

SEQ ID NO: 41 - protein, 32 aa, Homo sapiens
GIPDRFSGSSSGADRYLTISNLQSEDEADYYC

SEQ ID NO: 42 - protein, 11 aa, Homo sapiens
FGGGTKLTVLG

SEQ ID NO: 43 - protein, 21 aa, Homo sapiens
SVLTQSPSASASLGASVKLTC

SEQ ID NO: 44 - protein, 15 aa, Homo sapiens
WYQQQPGKGPRYLMK

SEQ ID NO: 45 - protein, 122 aa, Artificial Sequence
  Description of Artificial Sequence Synthetic Humanized 8G8 Immunoglobulin heavy chain polypeptide
EVQLVQSGAEVKKPGASVKVSCKASGYTFTNYGMNWVRQAPGQGLEWIGWINTYTGEPTYADDFKGRVTITRDTSTSTAYLELSSLRSEDTAVYYCARSWYYVSNYWYFDVWGQGTLVTVSS

SEQ ID NO: 46 - protein, 122 aa, Artificial Sequence
  Description of Artificial Sequence Synthetic Humanized 8G8 Immunoglobulin Heavy Chain polypeptide
EVQLVESGGGLVQPGGSLRLSCAASGYTFTNYGMNWVRQAPGKGLEWVGWINTYTGEPTYADDFKGRFTISRDNSKNTLYLQMNSLRAEDTAVYYCARSWYYVSNYWYFDVWGQGTLVTVSS

SEQ ID NO: 47 - protein, 122 aa, Artificial Sequence
  Description of Artificial Sequence Synthetic Humanized 8G8 Immunoglobulin Heavy Chain polypeptide
EVQLVQSGSELKKPGASVKVSCKASGYTFTNYGMNWVRQAPGQGLEWIGWINTYTGEPTYADDFKGRFVFSLDTSVSTAYLQISSLKAEDTAVYYCARSWYYVSNYWYFDVWGQGTLVTVSS

SEQ ID NO: 48 - protein, 116 aa, Artificial Sequence
  Description of Artificial Sequence Synthetic Humanized 8G8 Immunoglobulin Light Chain polypeptide
QPVLTQSPSASASLGASVKLTCTLSSQHSTYTIEWHQQQPGKGPRYLMKLKKDGSHSTGDGIPDRFSGSSSGADRYLTISNLQSEDEADYYCGVGDTIKEQFVYVFGGGTKLTVLG

SEQ ID NO: 49 - protein, 115 aa, Artificial Sequence
  Description of Artificial Sequence Synthetic Humanized 8G8 Immunoglobulin Light Chain (Mutated) polypeptide
SVLTQSPSASASLGASVKLTCTLSSQHSTYTIEWYQQQPGKGPRYLMKLKKDGSHSTGDGIPDRFSGSSSGADRYLTISNLQSEDEADYYCGVGDTIKEQFVYVFGGGTKLTVLG

SEQ ID NO: 50 - protein, 122 aa, Mus musculus
QIHLVQSGPELKKPGETVKISCKASGYTFTNYGMNWVKQAPGKGLKWMGWINTYTGEPTYADDFKGRFAFSLETSASTAYLQINNLKNEDMATYFCARSWYYVSNYWYFDVWGQGTLVTVSS

SEQ ID NO: 51 - protein, 116 aa, Mus musculus
QLVLTQSSSASFSLGASAKLTCTLSSQHSTYTIEWYQQQPLKPPKYVMELKKDGSHSTGDGIPDRFSGSSSGADRYLSISNIQPEDEAIYICGVGDTIKEQFVYVFGGGTKVTVLG

SEQ ID NO: 52 - protein, 117 aa, Homo sapiens
EVQLVQSGAEVKKPGASVKVSCKASGYTFTSYYIHWVRQAPGQGLEWIGWINPGSGNTNYAQKFQGRVTITRDTSTSTAYLELSSLRSEDTAVYYCARDTAAYFDYWGQGTLVTVSS

SEQ ID NO: 53 - protein, 117 aa, Homo sapiens
EVQLVESGGGLVQPGGSLRLSCAASGFTFSSYAMSWVRQAPGKGLEWVGAISSSGSSTYYADSVKGRFTISRDNSKNTLYLQMNSLRAEDTAVYYCARDTAAYFDYWGQGTLVTVSS

SEQ ID NO: 54 - protein, 117 aa, Homo sapiens
EVQLVQSGSELKKPGASVKVSCKASGYTFTSYYIHWVRQAPGQGLEWIGWINPGSGNTNYAQKFQGRFVFSLDTSVSTAYLQISSLKAEDTAVYYCARDTAAYFDYWGQGTLVTVSS

SEQ ID NO: 55 - protein, 112 aa, Homo sapiens
QPVLTQSPSASASLGASVKLTCTLSSGHSSYTIAWHQQQPGKGPRYLMKLNSDGSHSKGDGIPDRFSGSSSGADRYLTISNLQSEDEADYYCGTWGTGIVVFGGGTKLTVLG

SEQ ID NO: 56 - DNA, 345 bp, Artificial Sequence
  Description of Artificial Sequence Synthetic Humanized 8G8 Immunoglobulin Light Chain polynucleotide
tctgtgctgacccagagcccaagcgccagcgccagcctgggcgccagcgtgaaactgacctgcaccctgagcagccagcacagcacctacaccatcgaatggtatcagcagcagccaggcaaaggcccacgctacctgatgaaactgaaaaaagatggcagccacagcaccggcgatggcatcccagatcgcttcagcggcagcagcagcggcgccgatcgctacctgaccatcagcaacctgcagagcgaagatgaagccgattactactgcggcgtgggcgataccatcaaagaacagttcgtgtacgtgttcggcggcggtaccaaactgaccgtgctgggc

SEQ ID NO: 57 - protein, 12 aa, Mus musculus
LKKDGSHSTGDG

SEQ ID NO: 58 - protein, 12 aa, Artificial Sequence
  Description of Artificial Sequence Synthetic Mutated Mouse sequence
LKKDASHSTGDG

SEQ ID NO: 59 - protein, 12 aa, Artificial Sequence
  Description of Artificial Sequence Synthetic Mutated mouse sequence
LKKEGSHSTGDG

SEQ ID NO: 60 - protein, 12 aa, Artificial Sequence
  Description of Artificial Sequence Synthetic Mutated mouse sequence
LKKTGSHSTGDG

SEQ ID NO: 61 - protein, 12 aa, Artificial Sequence
  Description of Artificial Sequence Synthetic Mutated mouse sequence
LKKDGSHSTGDA

SEQ ID NO: 62 - protein, 12 aa, Artificial Sequence
  Description of Artificial Sequence Synthetic Mutated mouse sequence
LKKDGSHSTGEG

SEQ ID NO: 63 - protein, 12 aa, Artificial Sequence
  Description of Artificial Sequence Synthetic Mutated mouse sequence
LKKDGSHSTGTG

SEQ ID NO: 64 - protein, 12 aa, Artificial Sequence
  Description of Artificial Sequence Synthetic Mutated mouse sequence
LKKSGSHSTGDG

SEQ ID NO: 65 - protein, 12 aa, Artificial Sequence
  Description of Artificial Sequence Synthetic Mutated mouse sequence
LKKSGSHSTGSG

SEQ ID NO: 66 - protein, 12 aa, Artificial Sequence
  Description of Artificial Sequence Synthetic Mutated mouse sequence
LKKTGSHSTGDA

SEQ ID NO: 67 - protein, 32 aa, Artificial Sequence
  Description of Artificial Sequence Synthetic Mutant mouse FR3 polypeptide
AIPDRFSGSSSGADRYLSISNIQPEDEAIYIC

SEQ ID NO: 68 - protein, 32 aa, Artificial Sequence
  Description of Artificial Sequence Synthetic Mutated Human lambda 4 FR3 polypeptide
AIPDRFSGSSSGADRYLTISNLQSEDEADYYC

SEQ ID NO: 69 - protein, 109 aa, Homo sapiens
SYELTQPPSVSVSPGQTARITCQGDALRSYYASWYQQKPGQAPVLVIYKDNNRPSGIPERFSGSSSGNTATLTISGAQAEDEADYYCNSRDSSGNHVVFGGGTKLTVLG

SEQ ID NO: 70 - protein, 117 aa, Human cytomegalovirus
VEMAFAYALALFAAARQEEAGAQVSVPRALDRQAALLQIQEFMITCLSQTPPRTTLLLYPTAVDLAKRALWTPNQITDITSLVRLVYILSKQNQQHLIPQWALRQIADFALKLHKTH

SEQ ID NO: 71 - protein, 10 aa, Homo sapiens
GFTFSPYSVF

SEQ ID NO: 72 - protein, 18 aa, Homo sapiens
SSINSNSTYKYYADSVKG

SEQ ID NO: 73 - protein, 18 aa, Homo sapiens
SSINSDSRYKYYADSVKG

SEQ ID NO: 74 - protein, 18 aa, Homo sapiens
SSINSNSRYKYYADSVKG

SEQ ID NO: 75 - protein, 23 aa, Homo sapiens
ARDRSYYAFSSGSLSDYYYGLDV

SEQ ID NO: 76 - protein, 16 aa, Homo sapiens
RSSQSLLHTNGYNYLD

SEQ ID NO: 77 - protein, 7 aa, Homo sapiens
LASNRAS

SEQ ID NO: 78 - protein, 9 aa, Homo sapiens
MQALQIPRT

SEQ ID NO: 79 - protein, 25 aa, Homo sapiens
EEQVLESGGGLVKPGGSLRLSCAAS

SEQ ID NO: 80 - protein, 13 aa, Homo sapiens
WVRQAPGKGLEWV

SEQ ID NO: 81 - protein, 30 aa, Homo sapiens
RFTISRDNAENSIFLQMNSLRAEDTAVYYC

SEQ ID NO: 82 - protein, 11 aa, Homo sapiens
WGQGTLVTVSS

SEQ ID NO: 83 - protein, 23 aa, Homo sapiens
DIVMTQSPLSLSVTPGEPASISC

SEQ ID NO: 84 - protein, 15 aa, Homo sapiens
WYVQKPGQSPQLLIY

SEQ ID NO: 85 - protein, 32 aa, Homo sapiens
GVPDRFSGSGSGTDFTLKISRVETEDVGVYYC

SEQ ID NO: 86 - protein, 10 aa, Homo sapiens
FGQGTKVEIK

SEQ ID NO: 87 - protein, 130 aa, Artificial Sequence
  Description of Artificial Sequence Synthetic Affinity-matured human Ig Heavy Chain polypeptide
EEQVLESGGGLVKPGGSLRLSCAASGFTFSPYSVFWVRQAPGKGLEWVSSINSNSTYKYYADSVKGRFTISRDNAENSIFLQMNSLRAEDTAVYYCARDRSYYAFSSGSLSDYYYGLDVWGQGTLVTVSS

SEQ ID NO: 88 - protein, 130 aa, Artificial Sequence
  Description of Artificial Sequence Synthetic Affinity-matured human Ig Heavy Chain polypeptide
EEQVLESGGGLVKPGGSLRLSCAASGFTFSPYSVFWVRQAPGKGLEWVSSINSDSRYKYYADSVKGRFTISRDNAENSIFLQMNSLRAEDTAVYYCARDRSYYAFSSGSLSDYYYGLDVWGQGTLVTVSS

SEQ ID NO: 89 - protein, 130 aa, Artificial Sequence
  Description of Artificial Sequence Synthetic Affinity-matured human Ig Heavy Chain polypeptide
EEQVLESGGGLVKPGGSLRLSCAASGFTFSPYSVFWVRQAPGKGLEWVSSINSNSRYKYYADSVKGRFTISRDNAENSIFLQMNSLRAEDTAVYYCARDRSYYAFSSGSLSDYYYGLDVWGQGTLVTVSS

SEQ ID NO: 90 - protein, 112 aa, Homo sapiens
DIVMTQSPLSLSVTPGEPASISCRSSQSLLHTNGYNYLDWYVQKPGQSPQLLIYLASNRASGVPDRFSGSGSGTDFTLKISRVETEDVGVYYCMQALQIPRTFGQGTKVEIK

SEQ ID NO: 91 - protein, 18 aa, Homo sapiens
SSINSDSTYKYYADSVKG

SEQ ID NO: 92 - protein, 130 aa, Homo sapiens
EEQVLESGGGLVKPGGSLRLSCAASGFTFSPYSVFWVRQAPGKGLEWVSSINSDSTYKYYADSVKGRFTISRDNAENSIFLQMNSLRAEDTAVYYCARDRSYYAFSSGSLSDYYYGLDVWGQGTLVTVSS

SEQ ID NO: 93 - protein, 18 aa, Artificial Sequence
  Description of Artificial Sequence Synthetic Affinity-matured human Ig Heavy Chain peptide
SSIXSXSXYKYYADSVKG

SEQ ID NO: 94 - protein, 130 aa, Artificial Sequence
  Description of Artificial Sequence Synthetic Affinity-matured human Ig Heavy Chain polypeptide
EEQVLESGGGLVKPGGSLRLSCAASGFTFSPYSVFWVRQAPGKGLEWVSSIXSXSXYKYYADSVKGRFTISRDNAENSIFLQMNSLRAEDTAVYYCARDRSYYAFSSGSLSDYYYGLDVWGQGTLVTVSS

SEQ ID NO: 95 - protein, 22 aa, Homo sapiens
LEWVSSISSSSSYIYYADSVKG

SEQ ID NO: 96 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSSSTYKYYADSVKGRFTISRDNAENSIFLQMNSLRAEDTAVYYCARDRSYYAFSSGSLSDYYYGLDVWGQGTLVTVSS

SEQ ID NO: 97 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSISTYKYYADSVKGRFTISRDNAENSIFLQMNSLRAEDTAVYYCARDRSYYAFSSGSLSDYYYGLDVWGQGTLVTVSS

SEQ ID NO: 98 - protein, 130 aa, Artificial Sequence; identical to SEQ ID NO: 87
  Description of Artificial Sequence Synthetic Affinity matured human HC polypeptide

SEQ ID NO: 99 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSQSTYKYYADSVKGRFTISRDNAENSIFLQMNSLRAEDTAVYYCARDRSYYAFSSGSLSDYYYGLDVWGQGTLVTVSS

SEQ ID NO: 100 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSFSTYKYYADSVKGRFTISRDNAENSIFLQMNSLRAEDTAVYYCARDRSYYAFSSGSLSDYYYGLDVWGQGTLVTVSS

SEQ ID NO: 101 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSMSTYKYYADSVKGRFTISRDNAENSIFLQMNSLRAEDTAVYYCARDRSYYAFSSGSLSDYYYGLDVWGQGTLVTVSS

SEQ ID NO: 102 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSLSTYKYYADSVKGRFTISRDNAENSIFLQMNSLRAEDTAVYYCARDRSYYAFSSGSLSDYYYGLDVWGQGTLVTVSS

SEQ ID NO: 103 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSGSTYKYYADSVKGRFTISRDNAENSIFLQMNSLRAEDTAVYYCARDRSYYAFSSGSLSDYYYGLDVWGQGTLVTVSS

SEQ ID NO: 104 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSHSTYKYYADSVKGRFTISRDNAENSIFLQMNSLRAEDTAVYYCARDRSYYAFSSGSLSDYYYGLDVWGQGTLVTVSS

SEQ ID NO: 105 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSKSTYKYYADSVKGRFTISRDNAENSIFLQMNSLRAEDTAVYYCARDRSYYAFSSGSLSDYYYGLDVWGQGTLVTVSS

SEQ ID NO: 106 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSWSTYKYYADSVKGRFTISRDNAENSIFLQMNSLRAEDTAVYYCARDRSYYAFSSGSLSDYYYGLDVWGQGTLVTVSS

SEQ ID NO: 107 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSYSTYKYYADSVKGRFTISRDNAENSIFLQMNSLRAEDTAVYYCARDRSYYAFSSGSLSDYYYGLDVWGQGTLVTVSS

SEQ ID NO: 108 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSVSTYKYYADSVKGRFTISRDNAENSIFLQMNSLRAEDTAVYYCARDRSYYAFSSGSLSDYYYGLDVWGQGTLVTVSS

SEQ ID NO: 109 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSSSTYKYYADSVKGRFTISRDNAENSIFLQMNSLRAEDTAVYYCARDRSYYAFSSGSLSDYYYGLDVWGQGTLVTVSS

SEQ ID NO: 110 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSISTYKYYADSVKGRFTISRDNAENSIFLQMNSLRAEDTAVYYCARDRSYYAFSSGSLSDYYYGLDVWGQGTLVTVSS

SEQ ID NO: 111 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSNSTYKYYADSVKGRFTISRDNAENSIFLQMNSLRAEDTAVYYCARDRSYYAFSSGSLSDYYYGLDVWGQGTLVTVSS

SEQ ID NO: 112 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSQSTYKYYADSVKGRFTISRDNAENSIFLQMNSLRAEDTAVYYCARDRSYYAFSSGSLSDYYYGLDVWGQGTLVTVSS

SEQ ID NO: 113 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSFSTYKYYADSVKGRFTISRDNAENSIFLQMNSLRAEDTAVYYCARDRSYYAFSSGSLSDYYYGLDVWGQGTLVTVSS

SEQ ID NO: 114 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSMSTYKYYADSVKGRFTISRDNAENSIFLQMNSLRAEDTAVYYCARDRSYYAFSSGSLSDYYYGLDVWGQGTLVTVSS

SEQ ID NO: 115 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSLSTYKYYADSVKGRFTISRDNAENSIFLQMNSLRAEDTAVYYCARDRSYYAFSSGSLSDYYYGLDVWGQGTLVTVSS

SEQ ID NO: 116 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSGSTYKYYADSVKGRFTISRDNAENSIFLQMNSLRAEDTAVYYCARDRSYYAFSSGSLSDYYYGLDVWGQGTLVTVSS

SEQ ID NO: 117 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSHSTYKYYADSVKGRFTISRDNAENSIFLQMNSLRAEDTAVYYCARDRSYYAFSSGSLSDYYYGLDVWGQGTLVTVSS

SEQ ID NO: 118 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSKSTYKYYADSVKGRFTISRDNAENSIFLQMNSLRAEDTAVYYCARDRSYYAFSSGSLSDYYYGLDVWGQGTLVTVSS

SEQ ID NO: 119 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSWSTYKYYADSVKGRFTISRDNAENSIFLQMNSLRAEDTAVYYCARDRSYYAFSSGSLSDYYYGLDVWGQGTLVTVSS

SEQ ID NO: 120 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSYSTYKYYADSVKGRFTISRDNAENSIFLQMNSLRAEDTAVYYCARDRSYYAFSSGSLSDYYYGLDVWGQGTLVTVSS

SEQ ID NO: 121 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSVSTYKYYADSVKGRFTISRDNAENSIFLQMNSLRAEDTAVYYCARDRSYYAFSSGSLSDYYYGLDVWGQGTLVTVSS

SEQ ID NO: 122 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSSSRYKYYADSVKGRFTISRDNAENSIFLQMNSLRAEDTAVYYCARDRSYYAFSSGSLSDYYYGLDVWGQGTLVTVSS

SEQ ID NO: 123 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSISRYKYYADSVKGRFTISRDNAENSIFLQMNSLRAEDTAVYYCARDRSYYAFSSGSLSDYYYGLDVWGQGTLVTVSS

SEQ ID NO: 124 - protein, 130 aa, Artificial Sequence; identical to SEQ ID NO: 89
  Description of Artificial Sequence Synthetic Affinity matured human HC polypeptide

SEQ ID NO: 125 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSQSRYKYYADSVKGRFTISRDNAENSIFLQMNSLRAEDTAVYYCARDRSYYAFSSGSLSDYYYGLDVWGQGTLVTVSS

SEQ ID NO: 126 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSFSRYKYYADSVKGRFTISRDNAENSIFLQMNSLRAEDTAVYYCARDRSYYAFSSGSLSDYYYGLDVWGQGTLVTVSS

SEQ ID NO: 127 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSMSRYKYYADSVKGRFTISRDNAENSIFLQMNSLRAEDTAVYYCARDRSYYAFSSGSLSDYYYGLDVWGQGTLVTVSS

SEQ ID NO: 128 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSLSRYKYYADSVKGRFTISRDNAENSIFLQMNSLRAEDTAVYYCARDRSYYAFSSGSLSDYYYGLDVWGQGTLVTVSS

SEQ ID NO: 129 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSGSRYKYYADSVKGRFTISRDNAENSIFLQMNSLRAEDTAVYYCARDRSYYAFSSGSLSDYYYGLDVWGQGTLVTVSS

SEQ ID NO: 130 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSHSRYKYYADSVKGRFTISRDNAENSIFLQMNSLRAEDTAVYYCARDRSYYAFSSGSLSDYYYGLDVWGQGTLVTVSS

SEQ ID NO: 131 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSKSRYKYYADSVKGRFTISRDNAENSIFLQMNSLRAEDTAVYYCARDRSYYAFSSGSLSDYYYGLDVWGQGTLVTVSS

SEQ ID NO: 132 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSWSRYKYYADSVKGRFTISRDNAENSIFLQMNSLRAEDTAVYYCARDRSYYAFSSGSLSDYYYGLDVWGQGTLVTVSS

SEQ ID NO: 133 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSYSRYKYYADSVKGRFTISRDNAENSIFLQMNSLRAEDTAVYYCARDRSYYAFSSGSLSDYYYGLDVWGQGTLVTVSS

SEQ ID NO: 134 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSVSRYKYYADSVKGRFTISRDNAENSIFLQMNSLRAEDTAVYYCARDRSYYAFSSGSLSDYYYGLDVWGQGTLVTVSS

SEQ ID NO: 135 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSSSKYKYYADSVKGRFTISRDNAENSIFLQMNSLRAEDTAVYYCARDRSYYAFSSGSLSDYYYGLDVWGQGTLVTVSS

SEQ ID NO: 136 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSISKYKYYADSVKGRFTISRDNAENSIFLQMNSLRAEDTAVYYCARDRSYYAFSSGSLSDYYYGLDVWGQGTLVTVSS

SEQ ID NO: 137 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSNSKYKYYADSVKGRFTISRDNAENSIFLQMNSLRAEDTAVYYCARDRSYYAFSSGSLSDYYYGLDVWGQGTLVTVSS

SEQ ID NO: 138 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSQSKYKYYADSVKGRFTISRDNAENSIFLQMNSLRAEDTAVYYCARDRSYYAFSSGSLSDYYYGLDVWGQGTLVTVSS

SEQ ID NO: 139 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSFSKYKYYADSVKGRFTISRDNAENSIFLQMNSLRAEDTAVYYCARDRSYYAFSSGSLSDYYYGLDVWGQGTLVTVSS

SEQ ID NO: 140 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSMSKYKYYADSVKGRFTISRDNAENSIFLQMNSLRAEDTAVYYCARDRSYYAFSSGSLSDYYYGLDVWGQGTLVTVSS

SEQ ID NO: 141 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSLSKYKYYADSVKGRFTISRDNAENSIFLQMNSLRAEDTAVYYCARDRSYYAFSSGSLSDYYYGLDVWGQGTLVTVSS

SEQ ID NO: 142 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSGSKYKYYADSVKGRFTISRDNAENSIFLQMNSLRAEDTAVYYCARDRSYYAFSSGSLSDYYYGLDVWGQGTLVTVSS

SEQ ID NO: 143 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSHSKYKYYADSVKGRFTISRDNAENSIFLQMNSLRAEDTAVYYCARDRSYYAFSSGSLSDYYYGLDVWGQGTLVTVSS

SEQ ID NO: 144 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSKSKYKYYADSVKGRFTISRDNAENSIFLQMNSLRAEDTAVYYCARDRSYYAFSSGSLSDYYYGLDVWGQGTLVTVSS

SEQ ID NO: 145 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSWSKYKYYADSVKGRFTISRDNAENSIFLQMNSLRAEDTAVYYCARDRSYYAFSSGSLSDYYYGLDVWGQGTLVTVSS

SEQ ID NO: 146 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSYSKYKYYADSVKGRFTISRDNAENSIFLQMNSLRAEDTAVYYCARDRSYYAFSSGSLSDYYYGLDVWGQGTLVTVSS

SEQ ID NO: 147 - protein, 130 aa, Artificial Sequence
  Description of Artificial Sequence Synthetic Affinty matured human HC polypeptide
EEQVLESGGGLVKPGGSLRLSCAASGFTFSPYSVFWVRQAPGKGLEWVSSINSVSKYKYYADSVKGRFTISRDNAENSIFLQMNSLRAEDTAVYYCARDRSYYAFSSGSLSDYYYGLDVWGQGTLVTVSS

SEQ ID NO: 148 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSSSRYKYYADSVKGRFTISRDNAENSIFLQMNSLRAEDTAVYYCARDRSYYAFSSGSLSDYYYGLDVWGQGTLVTVSS

SEQ ID NO: 149 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSISRYKYYADSVKGRFTISRDNAENSIFLQMNSLRAEDTAVYYCARDRSYYAFSSGSLSDYYYGLDVWGQGTLVTVSS

SEQ ID NO: 150 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSNSRYKYYADSVKGRFTISRDNAENSIFLQMNSLRAEDTAVYYCARDRSYYAFSSGSLSDYYYGLDVWGQGTLVTVSS

SEQ ID NO: 151 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSQSRYKYYADSVKGRFTISRDNAENSIFLQMNSLRAEDTAVYYCARDRSYYAFSSGSLSDYYYGLDVWGQGTLVTVSS

SEQ ID NO: 152 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSFSRYKYYADSVKGRFTISRDNAENSIFLQMNSLRAEDTAVYYCARDRSYYAFSSGSLSDYYYGLDVWGQGTLVTVSS

SEQ ID NO: 153 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSMSRYKYYADSVKGRFTISRDNAENSIFLQMNSLRAEDTAVYYCARDRSYYAFSSGSLSDYYYGLDVWGQGTLVTVSS

SEQ ID NO: 154 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSLSRYKYYADSVKGRFTISRDNAENSIFLQMNSLRAEDTAVYYCARDRSYYAFSSGSLSDYYYGLDVWGQGTLVTVSS

SEQ ID NO: 155 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSGSRYKYYADSVKGRFTISRDNAENSIFLQMNSLRAEDTAVYYCARDRSYYAFSSGSLSDYYYGLDVWGQGTLVTVSS

SEQ ID NO: 156 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSHSRYKYYADSVKGRFTISRDNAENSIFLQMNSLRAEDTAVYYCARDRSYYAFSSGSLSDYYYGLDVWGQGTLVTVSS

SEQ ID NO: 157 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSKSRYKYYADSVKGRFTISRDNAENSIFLQMNSLRAEDTAVYYCARDRSYYAFSSGSLSDYYYGLDVWGQGTLVTVSS

SEQ ID NO: 158 - protein, 130 aa, Artificial Sequence; identical to SEQ ID NO: 132
  Description of Artificial Sequence Synthetic Affinity matured human HC polypeptide

SEQ ID NO: 159 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSYSRYKYYADSVKGRFTISRDNAENSIFLQMNSLRAEDTAVYYCARDRSYYAFSSGSLSDYYYGLDVWGQGTLVTVSS

SEQ ID NO: 160 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSVSRYKYYADSVKGRFTISRDNAENSIFLQMNSLRAEDTAVYYCARDRSYYAFSSGSLSDYYYGLDVWGQGTLVTVSS

SEQ ID NO: 161 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSSSKYKYYADSVKGRFTISRDNAENSIFLQMNSLRAEDTAVYYCARDRSYYAFSSGSLSDYYYGLDVWGQGTLVTVSS

SEQ ID NO: 162 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSISKYKYYADSVKGRFTISRDNAENSIFLQMNSLRAEDTAVYYCARDRSYYAFSSGSLSDYYYGLDVWGQGTLVTVSS

SEQ ID NO: 163 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSNSKYKYYADSVKGRFTISRDNAENSIFLQMNSLRAEDTAVYYCARDRSYYAFSSGSLSDYYYGLDVWGQGTLVTVSS

SEQ ID NO: 164 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSQSKYKYYADSVKGRFTISRDNAENSIFLQMNSLRAEDTAVYYCARDRSYYAFSSGSLSDYYYGLDVWGQGTLVTVSS

SEQ ID NO: 165 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSFSKYKYYADSVKGRFTISRDNAENSIFLQMNSLRAEDTAVYYCARDRSYYAFSSGSLSDYYYGLDVWGQGTLVTVSS

SEQ ID NO: 166 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSMSKYKYYADSVKGRFTISRDNAENSIFLQMNSLRAEDTAVYYCARDRSYYAFSSGSLSDYYYGLDVWGQGTLVTVSS

SEQ ID NO: 167 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSLSKYKYYADSVKGRFTISRDNAENSIFLQMNSLRAEDTAVYYCARDRSYYAFSSGSLSDYYYGLDVWGQGTLVTVSS

SEQ ID NO: 168 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSGSKYKYYADSVKGRFTISRDNAENSIFLQMNSLRAEDTAVYYCARDRSYYAFSSGSLSDYYYGLDVWGQGTLVTVSS

SEQ ID NO: 169 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSHSKYKYYADSVKGRFTISRDNAENSIFLQMNSLRAEDTAVYYCARDRSYYAFSSGSLSDYYYGLDVWGQGTLVTVSS

SEQ ID NO: 170 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSKSKYKYYADSVKGRFTISRDNAENSIFLQMNSLRAEDTAVYYCARDRSYYAFSSGSLSDYYYGLDVWGQGTLVTVSS

SEQ ID NO: 171 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSWSKYKYYADSVKGRFTISRDNAENSIFLQMNSLRAEDTAVYYCARDRSYYAFSSGSLSDYYYGLDVWGQGTLVTVSS

SEQ ID NO: 172 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSYSKYKYYADSVKGRFTISRDNAENSIFLQMNSLRAEDTAVYYCARDRSYYAFSSGSLSDYYYGLDVWGQGTLVTVSS

SEQ ID NO: 173 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSVSKYKYYADSVKGRFTISRDNAENSIFLQMNSLRAEDTAVYYCARDRSYYAFSSGSLSDYYYGLDVWGQGTLVTVSS

SEQ ID NO: 174 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSASTYKYYADSVKGRFTISRDNAENSIFLQMNSLRAEDTAVYYCARDRSYYAFSSGSLSDYYYGLDVWGQGTLVTVSS

SEQ ID NO: 175 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSASTYKYYADSVKGRFTISRDNAENSIFLQMNSLRAEDTAVYYCARDRSYYAFSSGSLSDYYYGLDVWGQGTLVTVSS

SEQ ID NO: 176 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSASRYKYYADSVKGRFTISRDNAENSIFLQMNSLRAEDTAVYYCARDRSYYAFSSGSLSDYYYGLDVWGQGTLVTVSS

SEQ ID NO: 177 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSASRYKYYADSVKGRFTISRDNAENSIFLQMNSLRAEDTAVYYCARDRSYYAFSSGSLSDYYYGLDVWGQGTLVTVSS

SEQ ID NO: 178 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSASKYKYYADSVKGRFTISRDNAENSIFLQMNSLRAEDTAVYYCARDRSYYAFSSGSLSDYYYGLDVWGQGTLVTVSS

SEQ ID NO: 179 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSASKYKYYADSVKGRFTISRDNAENSIFLQMNSLRAEDTAVYYCARDRSYYAFSSGSLSDYYYGLDVWGQGTLVTVSS

SEQ ID NO: 180 - protein, 130 aa, Artificial Sequence; identical to SEQ ID NO: 88
  Description of Artificial Sequence Synthetic Affinity matured human HC polypeptide

SEQ ID NO: 181 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSDSRYKYYADSVKGRFTISRDNAENSIFLQMNSLRAEDTAVYYCARDRSYYAFSSGSLSDYYYGLDVWGQGTLVTVSS

SEQ ID NO: 182 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSINSDSKYKYYADSVKGRFTISRDNAENSIFLQMNSLRAEDTAVYYCARDRSYYAFSSGSLSDYYYGLDVWGQGTLVTVSS

SEQ ID NO: 183 - protein, 130 aa, Artificial Sequence
  Description of Artificial Sequence Synthetic Affinity matured human HC polypeptide
EEQVLESGGGLVKPGGSLRLSCAASGFTFSPYSVFWVRQAPGKGLEWVSSISSDSKYKYYADSVKGRFTISRDNAENSIFLQMNSLRAEDTAVYYCARDRSYYAFSSGSLSDYYYGLDVWGQGTLVTVSS

SEQ ID NO: 184 - protein, 130 aa, Artificial Sequence; identical to SEQ ID NO: 92
  Description of Artificial Sequence Synthetic Affinity matured human HC polypeptide

SEQ ID NO: 185 - DNA, 366 bp, Artificial Sequence
  Description of Artificial Sequence Synthetic Humanized 8G8 Immunoglobulin heavy chain polypeptide
gaggttcagctggtgcagtctggcgccgaagtgaaaaaaccaggggcctcagtgaaagtgtcctgtaaagcttctggctacaccttcaccaactacggcatgaactgggtgcgtcaggccccgggtcagggcctggaatggatcggctggatcaacacctacaccggcgaaccaacctacgccgatgatttcaaaggccgtgtgactataacccgtgacacctccaccagcacagcctacctagaactgagcagcttaagaagcgaggacactgccgtctattattgcgcgcgtagctggtactacgtgagcaactactggtacttcgatgtgtggggtcaaggaaccctggtcaccgtctcctcg

SEQ ID NO: 186 - DNA, 345 bp, Artificial Sequence; identical to SEQ ID NO: 56
  Description of Artificial Sequence Synthetic Humanized 8G8 Immunoglobulin Light Chain (Mutated) polypeptide

SEQ ID NO: 187 - protein, 22 aa, Artificial Sequence
  Description of Artificial Sequence Synthetic IGHV3-21*01 peptide
LEWVSSISSSSSYIYYADSVKG

SEQ ID NO: 188 - protein, 22 aa, Artificial Sequence
  Description of Artificial Sequence Synthetic MSL-109 peptide
LEWVSSINSXSXYKYYADSVKG

SEQ ID NO: 189 - DNA, 15 bp, Artificial Sequence
  Description of Artificial Sequence Synthetic oligonucleotide
tcgcgcccgaagagg

SEQ ID NO: 190 - DNA, 17 bp, Artificial Sequence
  Description of Artificial Sequence Synthetic oligonucleotide
cggccggattgtggatt

SEQ ID NO: 191 - DNA, 23 bp, Artificial Sequence
  Description of Artificial Sequence Synthetic oligonucleotide
caccgacgaggattccgacaacg

SEQ ID NO: 192 - protein, 28 aa, Artificial Sequence
  Description of Artificial Sequence Synthetic peptide
TAEKNDYYRVPHYWDACSRALPDQTRYK

SEQ ID NO: 193 - protein, 28 aa, Artificial Sequence
  Description of Artificial Sequence Synthetic peptide
CPHVWMPPQTTPHDWKGSHTTSGLHRPH

SEQ ID NO: 194 - protein, 28 aa, Artificial Sequence
  Description of Artificial Sequence Synthetic peptide
SRALPDQTRYKYVEQLVDLTLNYHYDAS

SEQ ID NO: 195 - protein, 29 aa, Artificial Sequence
  Description of Artificial Sequence Synthetic peptide
HPHHEYLSDLYTPCSSSGRRDHSLERLTR

SEQ ID NO: 196 - protein, 28 aa, Artificial Sequence
  Description of Artificial Sequence Synthetic peptide
CPHVWMPPQTTPHDWKGSHTTSGLHRPH

SEQ ID NO: 197 - protein, 28 aa, Artificial Sequence
  Description of Artificial Sequence Synthetic peptide
CGLPPELKQTRVNLPAHSRYGPQAVDAR

SEQ ID NO: 198 - protein, 28 aa, Artificial Sequence
  Description of Artificial Sequence Synthetic peptide
VGLDQYLESVKKHKRLDVCRAKMGYMLQ

SEQ ID NO: 199 - protein, 25 aa, Artificial Sequence
  Description of Artificial Sequence Synthetic peptide
RQVVHNKLTSCNYNPLYLEADGRIR

SEQ ID NO: 200 - protein, 28 aa, Artificial Sequence
  Description of Artificial Sequence Synthetic peptide
RDYSVSRQVRLTFTEANNQTYTFCTHPN

SEQ ID NO: 201 - protein, 27 aa, Artificial Sequence
  Description of Artificial Sequence Synthetic peptide
SPWFTLTANQNPSPPWSKLTYPKPHDC

SEQ ID NO: 202 - protein, 28 aa, Artificial Sequence
  Description of Artificial Sequence Synthetic peptide
TAEKNDYYRVPHYWDACSRALPDQTRYK

SEQ ID NO: 203 - protein, 129 aa, Human cytomegalovirus
MRLCRVWLSVCLCAVVLGQCQRETAEKNDYYRVPHYWDACSRALPDQTRYKYVEQLVDLTLNYHYDASHGLDNFDVLKRINVTEVSLLISDFRRQNRRGGTNKRTTFNAAGSLAPHARSLEFSVRLFAN

SEQ ID NO: 204 - protein, 214 aa, Human cytomegalovirus
MLRLLLRHHFHCLLLCAVWATPCLASPWFTLTANQNPSPPWSKLTYPKPHDAATFYCPFLYPSPPRSPSQFSGFQRVSTGPECRNETLYLLYNREGQTLVERSSTWVKKVIWYLSGRNQTILQRMPRTASKPSDGNVQISVEDAKIFGAHMVPKQTKLLRFVVNDGTRYQMCVMKLESWAHVFRDYSVSFQVRLTFTEANNQTYTFCTHPNLIV

SEQ ID NO: 205 - protein, 171 aa, Human cytomegalovirus; identical to SEQ ID NO: 3

SEQ ID NO: 206 - protein, 743 aa, Human cytomegalovirus
MRPGLPFYLTVFAVYLLSHLPSQRYGADAASEALDPHAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFPQQLKAQDSLGQQPTTVPPPIDLSIPHVWMPPQTTPHDWKGSHTTSGLHRPHFNQTCILFDGHDLLFSTVTPCLHQGFYLMDELRYVKITLTEDFFVVTVSIDDDTPMLLIFGHLPRVLFKAPYQRDNFILRQTEKHELLVLVKKTQLNRHSYLKDSDFLDAALDFNYLDLSALLRNSFHRYAVDVLKSGRCQMLDRRTVEMAFAYALALFAAARQEEAGTEISIPRALDRQAALLQIQEFMITCLSQTPPRTTLLLYPTAVDLAKRALWTPDQITDITSLVRLVYILSKQNQQHLIPQWALRQIADFALQLHKTHLASFLSAFARQELYLMGSLVHSMLVHTTERREIFIVETGLCSLAELSHFTQLLAHPHHEYLSDLYTPCSSSGRRDHSLERLTRLFPDATVPATVPAALSILSTMQPSTLETFPDLFCLPLGESFSALTVSEHVSYVVTNQYLIKGISYPVSTTVVGQSLIITQTDSQSKCELTRNMHTTHSITAALNISLENCAFCQSALLEYDDTQGVINIMYMHDSDDVLFALDPYNEVVVSSPRTHYLMLLKNGTVLEVTDVVVDATDSRLLMMSVYALSAIIGIYLLYRMLKTC

SEQ ID NO: 207 - protein, 464 aa, Human cytomegalovirus
MGRKEDMRSISKLFFIISLTVLLFSIINCKVVRPPGRYWLGTVLSTIGKQKLDKFKLEILKQLEREPYTKYFNMTRQHVKNLTMNMTQFPQYYILAGPIRNDSITYLWFDFYSTQLRKPAKYVYSQYNHTAKTITFRPPSCGTVPSMTCLSEMLNVSKRNDTGEQGCGNFTTFNPMFFNVPRWNTKLYVGPTKVNVDSQTIYFLGLTALLLRYAQRNCTHSFYLVNAMSRNLFRVPKYINGTKLKNTMRKLKRKQAPVKEQLEKKTKKSQSTTTPYFSYTTSTALNVTTNATYRVTTSAKRIPTSTIAYRPDSSFMKSIMATQLRDLATWVYTTLRYRNEPFCKPDRNRTAVSEFMKNTHVLIRNETPYTIYGTLDMSSLYYNETMSVENETASDNNETTPTSPSTRFQKTFIDPLWDYLDSLLFLDKIRNFSLQLPAYGNLTPPEHRRAVNLSTLNSLWWWLQ

SEQ ID NO: 208 - protein, 278 aa, Human cytomegalovirus
MCRRPDCGFSFSPGPVVLLWCCLLLPIVSSVAVSVAPTAAEKVPAECPELTRRCLLGEVFQGDKYESWLRPLVNVTGRNGPLSQLIRYRPVTPEAANSVLLDDAFLDTLALLYNNPDQLRALLTLLSSDTAPRWMTVMRGYSECGDGSPAVYTCVDDLCRGYDLTRLSYGRSIFTEHVLGFELVPPSLFNVVVAIRNEATRTNRAVRLPVSTAAAPEGITLFYGLYNAVKEFCLRHQLDPPLLRHLDKYYAGLPPELKQTRVNLPAHSRYGPQAVDAR

SEQ ID NO: 209 - protein, 472 aa, Human cytomegalovirus
MGKKEMIMVKGIPKIMLLISITFLLLSLINCNVLVNSRGTRRSWPYTVLSYRGKEILKKQKEDILKRLMSTSSDGYRFLMYPSQQKFHAIVISMDKFPQDYILAGPIRNDSITHMWFDFYSTQLRKPAKYVYSEYNHTAHKITLRPPPCGTVPSMNCLSEMLNVSKRNDTGEKGCGNFTTFNPMFFNVPRWNTKLYIGSNKVNVDSQTIYFLGLTALLLRYAQRNCTRSFYLVNAMSRNLFRVPKYINGTKLKNTMRKLKRKQALVKEQPQKKNKKSQSTTTPYLSYTTSTAFNVTTNVTYSATAAVTRVATSTTGYRPDSNFMKSIMATQLRDLATWVYTTLRYRNEPFCKPDRNRTAVSEFMKNTHVLIRNETPYTIYGTLDMSSLYYNETMSVENETASDNNETTPTSPSTRFQRTFIDPLWDYLDSLLFLDKIRNFSLQLPAYGNLTPPEHRRAANLSTLNSLWWWSQ